SEQUENCE LISTING (79 sequences)

SEQ ID NO: 1 - protein, 7 aa, synthetic construct
  source 1..7: note = 2cVH CDR1
TSGMSVG

SEQ ID NO: 2 - protein, 16 aa, synthetic construct
  source 1..16: note = 2cVH CDR2
HIWWNNDKYYKPALKS

SEQ ID NO: 3 - protein, 12 aa, synthetic construct
  source 1..12: note = 2cVH CDR3
IYYGYRPYAMDY

SEQ ID NO: 4 - protein, 16 aa, synthetic construct
  source 1..16: note = 2cVL CDR1
RSSQSIVHSNGNTYLE

SEQ ID NO: 5 - protein, 7 aa, synthetic construct
  source 1..7: note = 2cVL CDR2
KVSNRFS

SEQ ID NO: 6 - protein, 9 aa, synthetic construct
  source 1..9: note = 2cVL CDR3
FQGSHVPWS

SEQ ID NO: 7 - protein, 122 aa, synthetic construct
  VARIANT 100..107: note = X can be any naturally occurring amino acid
  source 1..122: note = human germline VH framework
QVTLKESGPALVKPTQTLTLTCTFSGFSLSTSGMRVSWIRQPPGKALEWLARIDWDDDKFYSTSLKTRLTISKDTSKNQVVLTMTNMDPVDTATYYCARXXXXXXXXYFDYWGQGTLVTVSS

SEQ ID NO: 8 - protein, 114 aa, synthetic construct
  source 1..114: note = human germline VL framework
DIVMTQTPLSLPVTPGEPASISCRSSQSLLDSDDGNTYLEWYLQKPGQSPQLLIYTLSYRASGVPDRFSGSGSGTDFTLKISRVEAEDVGVYYCMQRIEFPWTFGQGTKVEIKR

SEQ ID NO: 9 - protein, 122 aa, synthetic construct
  source 1..122: note = VH of the humanized mAb hu2c antibody
QVTLKESGPALVKPTQTLTLTCTFSGFSLSTSGMSVGWIRQPPGKALEWLAHIWWNNDKYYKPALKSRLTISKDTSKNQVVLTMTNMDPVDTATYYCARIYYGYRPYAMDYWGQGTLVTVSS

SEQ ID NO: 10 - protein, 113 aa, synthetic construct
  source 1..113: note = VL of the humanized mAb hu2c antibody
DIVMTQTPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPWSFGQGTKVEIKR

SEQ ID NO: 11 - protein, 903 aa, Herpes Simplex Virus 1
  source 1..903: note = strain F
MRQGAARGCRWFVVWALLGLTLGVLVASAAPSSPGTPGVAAATQAANGGPATPAPPAPGPAPTGDTKPKKNKKPKNPPPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 12 - protein, 904 aa, Herpes Simplex Virus 1
  source 1..904: note = strain KOS
MHQGAPSWGRRWFVVWALLGLTLGVLVASAAPTSPGTPGVAAATQAANGGPATPAPPPLGAAPTGDPKPKKNKKPKNPTPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTTYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYQANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASVTVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 13 - protein, 904 aa, Herpes Simplex Virus 1
  source 1..904: note = strain gC-39-R6
MHQGAPSWGRRWFVVWALLGLTLGVLVASAAPSSPGTPGVAAATQAANGGPATPAPPALGAAPTGDPKPKKNKKPKNPTPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQHHVNDMLGRVAIAWCELQNHELTLWNEARKLNPNAIASVTVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 14 - protein, 904 aa, Herpes Simplex Virus 2
  source 1..904: note = strain HG52
MRGGGLICALVVGALVAAVASAAPAAPAAPRASGGVAATVAANGGPASRPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINTKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTEYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 15 - protein, 904 aa, Herpes Simplex Virus 2
  source 1..904: note = strain 333
MRGGGLICALVVGALVAAVASAAPAAPAAPRASGGVAATVAANGGPASRPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVTCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLGVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 16 - protein, 901 aa, Herpes Simplex Virus 2
  source 1..901: note = strain MMA
MRGGGLICALVVGALVAAVASAAPAAPRASGGVAATVAANGGPASRPPPVPSPATTRARKRKTKKPPERPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKAQATSPTTRNLLTTPKFTVAWDWVPKRPAVCTMTKWQEVDEMLRAEYGGSFRFSSDAISTTFTTNLTEYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 17 - protein, 375 aa, synthetic construct
  source 1..375: note = Fc gamma RI/CD64 transcript variant 1
MWFLTTLLLWVPVDGQVVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 18 - protein, 374 aa, synthetic construct
  source 1..374: note = Fc gamma RI/CD64 transcript variant 2
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 19 - protein, 367 aa, synthetic construct
  source 1..367: note = Fc gamma RI/CD64 transcript variant 3
MWFLTTLLLWDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 20 - protein, 357 aa, synthetic construct
  source 1..357: note = Fc gamma RI/CD64 transcript variant 4
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLVGIMFLVNTVLWVTIRKELKRKKKWDLEISLDSGHEKKVISSLQEDRHLEEELKCQEQKEEQLQEGVHRKEPQGAT

SEQ ID NO: 21 - protein, 351 aa, synthetic construct
  source 1..351: note = Fc gamma RI/CD64 transcript variant 5
MGKWPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 22 - protein, 290 aa, synthetic construct
  source 1..290: note = Fc gamma RI/CD64 transcript variant 6
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 23 - protein, 289 aa, synthetic construct
  source 1..289: note = Fc gamma RI/CD64 transcript variant 7
MWFLTTLLLWVPVDGQVGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 24 - protein, 282 aa, synthetic construct
  source 1..282: note = Fc gamma RI/CD64 transcript variant 8
MWFLTTLLLWGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 25 - protein, 265 aa, synthetic construct
  source 1..265: note = Fc gamma RI/CD64 transcript variant 9
MWFLTTLLLWGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLVGIMFLVNTVLWVTIRKELKRKKKWDLEISLDSGHEKKVISSLQEDRHLEEELKCQEQKEEQLQEGVHRKEPQGAT

SEQ ID NO: 26 - protein, 317 aa, synthetic construct
  source 1..317: note = Fc gamma RIIa/CD32a transcript variant 1
MTMETQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 27 - protein, 316 aa, synthetic construct
  source 1..316: note = Fc gamma RIIa/CD32a transcript variant 2
MTMETQMSQNVCPRNLWLLQPLTVLLLLASADSQAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 28 - protein, 276 aa, synthetic construct
  source 1..276: note = Fc gamma RIIa/CD32a transcript variant 3
MTMETQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQANSTDPVKAAQFEPPGRQMIAIRKRQLEETNNDYETADGGYMTLNPRAPTDDDKNIYLTLPPNDHVNSNN

SEQ ID NO: 29 - protein, 275 aa, synthetic construct
  source 1..275: note = Fc gamma RIIa/CD32a transcript variant 4
MTMETQMSQNVCPRNLWLLQPLTVLLLLASADSQAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQANSTDPVKAAQFEPPGRQMIAIRKRQLEETNNDYETADGGYMTLNPRAPTDDDKNIYLTLPPNDHVNSNN

SEQ ID NO: 30 - protein, 323 aa, synthetic construct
  source 1..323: note = Fc gamma RIIc/CD32c transcript variant 1
MGILSFLPVLATESDWADCKSPQPWGHMLLWTAVLFLAPVAGTPAAPPKAVLKLEPQWINVLQEDSVTLTCRGTHSPESDSIPWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANSTDPVKAAQFEPPGRQMIAIRKRQPEETNNDYETADGGYMTLNPRAPTDDDKNIYLTLPPNDHVNSNN

SEQ ID NO: 31 - protein, 254 aa, synthetic construct
  source 1..254: note = Fc gamma RIIIa/CD16a transcript variant 1
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 32 - protein, 358 aa, synthetic construct
  source 1..358: note = Fc gamma RIIIa/CD16a transcript variant 2
MAEGTLWQILCVSSDAQPQTFEGVKGADPPTLPPGSFLPGPVLWWGSLARLQTEKSDEVSRKGNWWVTEMGGGAGERLFTSSCLVGLVPLGLRISLVTCPLQCGIMWQLLLPTALLLLVSAGMRTDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 33 - protein, 253 aa, synthetic construct
  source 1..253: note = Fc gamma RIIIa/CD16a transcript variant 5
MWQLLLPTALLLLVSAGMRTDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 34 - protein, 273 aa, synthetic construct
  source 1..273: note = Fc gamma RIIIa/CD16a transcript variant 7
MAEGTLWQILCVSSDAQPQTFEGVKGADPPTLPPGSFLPGPVLWWGSLARLQTEKSDEVSRKGNWWVTEMGGGAGERLFTSSCLVGLVPLGLRISLVTCPLQCGIMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGLAVSTISSFFPPGYQVSFCLVMVLLFAVDTGLYFSVKTNIRSSTRDWKDHKFKWRKDPQDK

SEQ ID NO: 35 - protein, 233 aa, synthetic construct
  source 1..233: note = Fc gamma RIIIb/CD16b transcript variant 2
MWQLLLPTALLLLVSAGMRTEDLPKAVVFLEPQWYSVLEKDSVTLKCQGAYSPEDNSTQWFHNENLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 36 - protein, 232 aa, synthetic construct
  source 1..232: note = Fc gamma RIIIb/CD16b transcript variant 3
MWQLLLPTALLLLVSAGMRTDLPKAVVFLEPQWYSVLEKDSVTLKCQGAYSPEDNSTQWFHNENLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 37 - protein, 216 aa, synthetic construct
  source 1..216: note = Fc gamma RIIIb/CD16b transcript variant 4
MRTEDLPKAVVFLEPQWYSVLEKDSVTLKCQGAYSPEDNSTQWFHNENLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 38 - protein, 130 aa, synthetic construct
  source 1..130: note = Fc gamma RIIIb/CD16b transcript variant 5
MRTEDLPKAVVFLEPQWYSVLEKDSVTLKCQGAYSPEDNSTQWFHNENLISSQASSYFIDAATVNDSGEYRCQTNLSTLSDPVQLEVHIGLAVSTISSFSPPGYQVSFCLVMVLLFAVDTGLYFSVKTNI

SEQ ID NO: 39 - protein, 310 aa, synthetic construct
  source 1..310: note = Fc gamma RIIb/CD32b transcript variant 1
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISALPGYPECREMGETLPEKPANPTNPDEADKVGAENTITYSLLMHPDALEEPDDQNRI

SEQ ID NO: 40 - protein, 290 aa, synthetic construct
  source 1..290: note = Fc gamma RIIb/CD32b transcript variant 2
MGILSFLPVLATESDWADCKSPQPWGHMLLWTAVLFLAPVAGTPAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANPTNPDEADKVGAENTITYSLLMHPDALEEPDDQNRI

SEQ ID NO: 41 - protein, 291 aa, synthetic construct
  source 1..291: note = Fc gamma RIIb/CD32b transcript variant 3
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANPTNPDEADKVGAENTITYSLLMHPDALEEPDDQNRI

SEQ ID NO: 42 - protein, 309 aa, synthetic construct
  source 1..309: note = Fc gamma RIIb/CD32b transcript variant 4
MGILSFLPVLATESDWADCKSPQPWGHMLLWTAVLFLAPVAGTPAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISALPGYPECREMGETLPEKPANPTNPDEADKVGAENTITYSLLMHPDALEEPDDQNRI

SEQ ID NO: 43 - protein, 303 aa, synthetic construct
  source 1..303: note = Fc gamma RIIb/CD32b transcript variant 5
MGILSFLPVLATESDWADCKSPQPWGHMLLWTAVLFLAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISALPGYPECREMGETLPEKPANPTNPDEADKVGAENTITYSLLMHPDALEEPDDQNRI

SEQ ID NO: 44 - protein, 302 aa, synthetic construct
  source 1..302: note = Fc gamma RIIb/CD32b transcript variant 6
MGILSFLPVLATESDWADCKSPQPWGHMLLWTAVLFLAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISALPGYPECREMGETLPEKPANPTNPDEADKVGAENTITYSLLMHPDALEEPDDQNRI

SEQ ID NO: 45 - protein, 284 aa, synthetic construct
  source 1..284: note = Fc gamma RIIb/CD32b transcript variant 7
MGILSFLPVLATESDWADCKSPQPWGHMLLWTAVLFLAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANPTNPDEADKVGAENTITYSLLMHPDALEEPDDQNRI

SEQ ID NO: 46 - protein, 283 aa, synthetic construct
  source 1..283: note = Fc gamma RIIb/CD32b transcript variant 8
MGILSFLPVLATESDWADCKSPQPWGHMLLWTAVLFLAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANPTNPDEADKVGAENTITYSLLMHPDALEEPDDQNRI

SEQ ID NO: 47 - protein, 273 aa, synthetic construct
  source 1..273: note = Fc gamma RIIb/CD32b transcript variant 9
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISALPGYPECREMGETLPEKPG

SEQ ID NO: 48 - protein, 265 aa, synthetic construct
  source 1..265: note = Fc gamma RIIb/CD32b transcript variant 10
MGILSFLPVLATESDWADCKSPQPWGHMLLWTAVLFLAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISALPGYPECREMGETLPEKPG

SEQ ID NO: 49 - protein, 254 aa, synthetic construct
  source 1..254: note = Fc gamma RIIb/CD32b transcript variant 11
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISG

SEQ ID NO: 50 - protein, 247 aa, synthetic construct
  source 1..247: note = Fc gamma RIIb/CD32b transcript variant 12
MGILSFLPVLATESDWADCKSPQPWGHMLLWTAVLFLAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISG

SEQ ID NO: 51 - protein, 365 aa, synthetic construct
  source 1..365: note = FcRn
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSVLVVGIVIGVLLLTAAAVGGALLWRRMRSGLPAPWISLRGDDTGVLLPTPGEAQDADLKDVNVIPATA

SEQ ID NO: 52 - protein, 475 aa, synthetic construct [sequence omitted]

SEQ ID NO: 53 - protein, 227 aa, synthetic construct
  source 1..227: note = HDIT101 hinge*-CH2-CH3 (Fc domain)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 227 aa, synthetic construct
  source 1..227: note = HDIT101 hinge*-CH2-CH3 G236A (Fc domain containing G236A substitution)
DKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 227 aa, synthetic construct
  source 1..227: note = HDIT101 hinge*-CH2-CH3 G236A/S239D/I332E (Fc domain containing G236A/S239D/I332E substitution)
DKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 935 aa, synthetic construct
  source 1..935: note = HSV-1F gB extracellular domain fused to HDIT101 hinge*-CH2-CH3
APSSPGTPGVAAATQAANGGPATPAPPAPGPAPTGDTKPKKNKKPKNPPPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAATRSGAAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 935 aa, synthetic construct
  source 1..935: note = HSV-1F gB extracellular domain fused to HDIT101 hinge*-CH2-CH3 G236A
APSSPGTPGVAAATQAANGGPATPAPPAPGPAPTGDTKPKKNKKPKNPPPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAATRSGAAASDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 935 aa, synthetic construct
  source 1..935: note = HSV-1F gB extracellular domain fused to HDIT101 hinge*-CH2-CH3 G236A/S239D/I332E
APSSPGTPGVAAATQAANGGPATPAPPAPGPAPTGDTKPKKNKKPKNPPPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAATRSGAAASDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 937 aa, synthetic construct
  source 1..937: note = HSV-2G gB extracellular domain fused to HDIT101 hinge*-CH2-CH3
APAAPRASGGVAATVAANGGPASRPPPVPSPATTRARKRKTKKPPERPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAATRSGAAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 937 aa, synthetic construct
  source 1..937: note = HSV-2G gB extracellular domain fused to HDIT101 hinge*-CH2-CH3 G236A
APAAPRASGGVAATVAANGGPASRPPPVPSPATTRARKRKTKKPPERPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAATRSGAAASDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 937 aa, synthetic construct
  source 1..937: note = HSV-2G gB extracellular domain fused to HDIT101 hinge*-CH2-CH3 G236A/S239D/I332E
APAAPRASGGVAATVAANGGPASRPPPVPSPATTRARKRKTKKPPERPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAATRSGAAASDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 944 aa, synthetic construct
  source 1..944: note = HSV-1F gB extracellular domain fused to HIV-1 Tat peptide and HDIT101 hinge*-CH2-CH3
APSSPGTPGVAAATQAANGGPATPAPPAPGPAPTGDTKPKKNKKPKNPPPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAATRYGRKKRRQRRRAAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 944 aa, synthetic construct
  source 1..944: note = HSV-1F gB extracellular domain fused to HIV-1 Tat peptide and HDIT101 hinge*-CH2-CH3 G236A
APSSPGTPGVAAATQAANGGPATPAPPAPGPAPTGDTKPKKNKKPKNPPPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAATRYGRKKRRQRRRAAASDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 944 aa, synthetic construct
  source 1..944: note = HSV-1F gB extracellular domain fused to HIV-1 Tat peptide and HDIT101 hinge*-CH2-CH3 G236A/S239D/I332E
APSSPGTPGVAAATQAANGGPATPAPPAPGPAPTGDTKPKKNKKPKNPPPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAATRYGRKKRRQRRRAAASDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 946 aa, synthetic construct
  source 1..946: note = HSV-2G gB extracellular domain fused to HIV-1 Tat peptide and HDIT101 hinge*-CH2-CH3
APAAPRASGGVAATVAANGGPASRPPPVPSPATTRARKRKTKKPPERPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAATRYGRKKRRQRRRAAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 946 aa, synthetic construct
  source 1..946: note = HSV-2G gB extracellular domain fused to HIV-1 Tat peptide and HDIT101 hinge*-CH2-CH3 G236A
APAAPRASGGVAATVAANGGPASRPPPVPSPATTRARKRKTKKPPERPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAATRYGRKKRRQRRRAAASDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 946 aa, synthetic construct
  source 1..946: note = HSV-2G gB extracellular domain fused to HIV-1 Tat peptide and HDIT101 hinge*-CH2-CH3 G236A/S239D/I332E
APAAPRASGGVAATVAANGGPASRPPPVPSPATTRARKRKTKKPPERPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAATRYGRKKRRQRRRAAASDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 943 aa, synthetic construct
  source 1..943: note = HSV-1F gB extracellular domain fused to HIV-1 PTD-4 peptide and HDIT101 hinge*-CH2-CH3
APSSPGTPGVAAATQAANGGPATPAPPAPGPAPTGDTKPKKNKKPKNPPPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAATRYARAAARQARAAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 943 aa, synthetic construct
  source 1..943: note = HSV-1F gB extracellular domain fused to HIV-1 PTD-4 peptide and HDIT101 hinge*-CH2-CH3 G236A
APSSPGTPGVAAATQAANGGPATPAPPAPGPAPTGDTKPKKNKKPKNPPPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAATRYARAAARQARAAASDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 943 aa, synthetic construct
  source 1..943: note = HSV-1F gB extracellular domain fused to HIV-1 PTD-4 peptide and HDIT101 hinge*-CH2-CH3 G236A/S239D/I332E
APSSPGTPGVAAATQAANGGPATPAPPAPGPAPTGDTKPKKNKKPKNPPPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAATRYARAAARQARAAASDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 945 aa, synthetic construct
  source 1..945: note = HSV-2G gB extracellular domain fused to HIV-1 PTD-4 peptide and HDIT101 hinge*-CH2-CH3
APAAPRASGGVAATVAANGGPASRPPPVPSPATTRARKRKTKKPPERPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAATRYARAAARQARAAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 945 aa, synthetic construct
  source 1..945: note = HSV-2G gB extracellular domain fused to HIV-1 PTD-4 peptide and HDIT101 hinge*-CH2-CH3 G236A
APAAPRASGGVAATVAANGGPASRPPPVPSPATTRARKRKTKKPPERPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAATRYARAAARQARAAASDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 945 aa, synthetic construct
  source 1..945: note = HSV-2G gB extracellular domain fused to HIV-1 PTD-4 peptide and HDIT101 hinge*-CH2-CH3 G236A/S239D/I332E
APAAPRASGGVAATVAANGGPASRPPPVPSPATTRARKRKTKKPPERPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAATRYARAAARQARAAASDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 948 aa, synthetic construct
  source 1..948: note = HSV-1F gB extracellular domain fused to IgG1 hinge*-CH2-CH3
MRAWIFFLLCLAGRALAAPSSPGTPGVAAATQAANGGPATPAPPAPGPAPTGDTKPKKNKKPKNPPPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAAAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEVTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 954 aa, synthetic construct
  source 1..954: note = HSV-2G gB extracellular domain fused to IgG1 hinge*-CH2-CH3
MRAWIFFLLCLAGRALAAPLDAPAAPRASGGVAATVAANGGPASRPPPVPSPATTRARKRKTKKPPERPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAAAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEVTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 987 aa, synthetic construct
  source 1..987: note = HSV-1F gB extracellular domain fused to IgG1 hinge*-CH2-CH3-StrepTag
MRAWIFFLLCLAGRALAAPSSPGTPGVAAATQAANGGPATPAPPAPGPAPTGDTKPKKNKKPKNPPPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAAAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEVTKNQVSLTCLVKGFYPSDIAVEWESNGQPENNYKTTPPVLDSDGSFFLYSKLTVDKSRWQQGNVFSCSVMHEALHNHYTQKSLSLSPGKGSLVPRGVRSAWSHPQFEKGGGSGGGSGGGSWSHPQFEK

SEQ ID NO: 77 - protein, 993 aa, synthetic construct
  source 1..993: note = HSV-2G gB extracellular domain fused to IgG1 hinge*-CH2-CH3-StrepTag
MRAWIFFLLCLAGRALAAPLDAPAAPRASGGVAATVAANGGPASRPPPVPSPATTRARKRKTKKPPERPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAAAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEVTKNQVSLTCLVKGFYPSDIAVEWESNGQPENNYKTTPPVLDSDGSFFLYSKLTVDKSRWQQGNVFSCSVMHEALHNHYTQKSLSLSPGKGSLVPRGVRSAWSHPQFEKGGGSGGGSGGGSWSHPQFEK

SEQ ID NO: 78 - protein, 758 aa, synthetic construct
  source 1..758: note = HSV-1F gB extracellular domain fused to StrepTag
MRAWIFFLLCLAGRALAAPLAAPSSPGTPGVAAATQAANGGPATPAPPAPGPAPTGDTKPKKNKKPKNPPPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAGSLVPRGSAWSHPQFEKGGGSGGGSGGGSWSHPQFEK

SEQ ID NO: 79 - protein, 760 aa, synthetic construct
  source 1..760: note = HSV-2G gB extracellular domain fused to StrepTag
MRAWIFFLLCLAGRALAAPLAAPAAPRASGGVAATVAANGGPASRPPPVPSPATTRARKRKTKKPPERPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTQYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAGSLVPRGSAWSHPQFEKGGGSGGGSGGGSWSHPQFEK

(19) United States
(12) Patent Application Publication
(10) Pub. No.: US 2026/0001939 A1
(43) Pub. Date: Jan. 1, 2026

(54) VACCINE COMPRISING AN ANTIBODY OR AN FC-CONTAINING FUSION PROTEIN COMPRISING AN FC PART OF AN ANTIBODY

(71) Applicant: HEIDELBERG IMMUNOTHERAPEUTICS GMBH, Heidelberg (DE)

(72) Inventors: Michaela ARNDT, Mannheim (DE); Torsten SCHALLER, Leimen (DE); Narges SEYFIZADEH, Dossenheim (DE); Christian MÜLLER, Darmstadt (DE)

(21) Appl. No.: 18/842,490

(22) PCT Filed: Mar. 1, 2023
(86) PCT No.: PCT/EP2023/055223

§ 371 Date: Aug. 29, 2024

(30) Foreign Application Priority Data
Mar. 2, 2022 (EP) 22159679.4

(51) Int. Cl. C07K 16/08; A61K 39/00; A61P 37/04

(52) U.S. Cl. CPC C07K 16/087; A61P 37/04; A61K 2039/505; C07K 2317/52

(57) ABSTRACT

Described is a vaccine for use in actively immunising a subject against an infectious disease or a malignant disease, said vaccine comprising: (a) an antibody against an antigen correlated with said infectious disease or malignant disease; or (b) an Fc-containing fusion protein comprising an Fc part of an antibody fused to an antigen correlated with said infectious disease or malignant disease, wherein said Fc part is capable of binding a receptor present on or in an antigen presenting cell (APC) selected from the group consisting of Type I: activatory FcγRI, FcγRIIa, FcγRIIc, FcγRIIIa, FcγRIIIb, and inhibitory FcγRIIb; and Type II: neonatal FcR (FcRn) and cytosolic TRIM21. Moreover, described is a vaccine for use in actively immunising a subject against an HSV-associated disease; and (a) wherein said antibody is an anti-HSV antibody and said subject suffers from an acute HSV-associated disease, preferably an acute HSV infection; or (b) wherein in said Fc-containing fusion protein comprising an Fc part of an antibody fused to an antigen, the antigen correlates with an HSV-associated disease.

The instant application is a national phase application under 35 U.S.C. § 371, of International Application No. PCT/EP2023/055223 entitled “VACCINE COMPRISING AN ANTIBODY OR AN FC-CONTAINING FUSION PROTEIN COMPRISING AN FC PART OF AN ANTIBODY” filed Mar. 1, 2023, which claims benefit of priority to European Patent Application No. 22159679.4 filed on Mar. 2, 2022, the entire contents of each of which is hereby incorporated by reference in its entirety.

The sequence listing associated with this application is filed in electronic format and is hereby incorporated by reference into the specification in its entirety. A replacement sequence listing submitted as an XML file entitled “1111_119_AF1071 US S3_Replacement sequence listing.xml” with a file size of 123 kB and a production date of Mar. 17, 2025, is hereby incorporated by reference into the specification in its entirety.

The present invention relates to a vaccine for use in actively immunising a subject against an infectious disease or a malignant disease, said vaccine comprising: (a) an antibody against an antigen correlated with said infectious disease or malignant disease; or (b) an Fc-containing fusion protein comprising an Fc part of an antibody fused to an antigen correlated with said infectious disease or malignant disease, wherein said Fc part is capable of binding a receptor present on or in an antigen presenting cell (APC) selected from the group consisting of Type I: activatory FcγRI, FcγRIIa, FcγRIIc, FcγRIIIa, FcγRIIIb, and inhibitory FcγRIIb; and Type II: neonatal FcR (FcRn) and cytosolic TRIM21. Moreover, the present invention relates to a vaccine for use in actively immunising a subject against an HSV-associated disease; and (a) wherein said antibody is an anti-HSV antibody and said subject suffers from an acute HSV-associated disease, preferably an acute HSV infection; or (b) wherein in said Fc-containing fusion protein comprising an Fc part of an antibody fused to an antigen, the antigen correlates with an HSV-associated disease.

A vaccine constitutes a biological agent that is used to induce an immune response to protect the vaccinated individual from an infection or the development of disease, often induced by a pathogenic microbial agent or a cancer cell. To induce the immune response, the vaccine needs to include a signature that corresponds to the target to which an immune response is aimed to be induced (e.g., viral proteins). Actively immunizing approaches are divided in live and non-live vaccines. Live vaccines are usually attenuated pathogens, while non-live vaccines contain or encode for components of the pathogen or entire pathogens that were inactivated [1]. These components can also be modified, e.g., by the expression as fusion proteins with effector domains, to modulate immune responses.

Live vaccines have been used since the discovery of vaccination and since then have been developed to protect against tuberculosis (BCG), typhoid, poliomyelitis, measles, mumps and rubella (MMR combined vaccine), rotavirus, smallpox, varicella zoster, influenza, Japanese encephalitis and yellow fever virus infection. The principle of attenuating pathogenicity and virus replicative fitness is allowing for the immune response to build up before vaccine strains revert to pathogenic wild type strains, however cases of evolution of wild type poliovirus after vaccination with live-attenuated poliovirus Sabin vaccine strain for example are well known and documented.

The group of non-live vaccines is increasing rapidly with newer available technologies. Licensed vaccines using whole inactivated pathogens include whole-cell pertussis, poliovirus, influenza virus, Japanese encephalitis virus, hepatitis A virus and rabies virus. Subunit vaccines with purified protein, recombinant natural protein or artificial fusion constructs, polysaccharides or peptides derived from the pathogen have been developed against pertussis, influenza, hepatitis B, meningococcal, pneumococcal, typhoid, SARS-CoV-2 and hepatitis A. Protein-polysaccharide conjugates were developed against Haemophilus influenzae type B, pneumococcal, meningococcal, typhoid. Virus like-particle vaccines in which pathogenic structures are presented on the surface of non-infectious particles derived from viruses were developed against Ebolavirus, hepatitis B virus, malaria and human papillomavirus. Similarly, epitope-coated polymers have been demonstrated to elicit neutralizing antibodies, e.g. to Plasmodium falciparum: sporozoites [2].

Viral vectors have been used to encode and deliver the pathogenic information of the antigen for vaccination. Examples for adenoviral vector-based vaccinations are Ebolavirus (ChAd3-EBO-Z) as well as SARS-CoV-2 (e.g. Ad5-nCoV, Ad26.COV2-S, Sputnik V, ChAdOX1-nCoV). In addition, recombinant vesicular stomatitis virus (rVSV), poxviruses (modified vaccinia Ankara), adeno-associated virus (AAV), retroviruses, cytomegalovirus and Sendai virus have been developed as viral vectors for vaccination. Based on the approach to deliver the antigen not directly, but encoded within a nucleic acid that is injected in a vehicle, mRNA and DNA have been used as vaccine platforms [3]. The SARS-CoV-2 pandemic led to the approval of the first mRNA vaccine encoding a modified viral Spike protein. The mRNA is delivered via lipid particles into the cytoplasm, and the cellular machinery translates this message into the viral protein. RNA vaccine approaches have been also experimentally tested for other infectious diseases, e.g. Ebolavirus, as well as in cancer therapy by the expression of cancer-related neoantigens. In addition, the first DNA vaccine was developed against SARS-CoV-2 (ZyCoV-D) and associated coronavirus disease (COVID) and approved in India for emergency use [4]. DNA vaccines have several advantages, including easy, fast and cheap production, stability as well as the possibility for fast adaptation and have been experimentally developed against several pathogens, including malarial parasites and mycoplasmas, as well as influenza virus, hepatitis B virus, human immunodeficiency virus, rabies virus, lymphocytic chorio-meningitis virus.

The aim of a vaccine is to induce long-lasting and humoral and cellular immune responses conferred by memory B and T cells that can be readily reactivated. B cells are activated to secrete antibodies when their B cell receptor interacts with Ag. In humans five major antibody isotypes are categorized based on their unique Fc fragments as IgM, IgG, IgA, IgD, and IgE. Each class interacts with specific Fc receptors (FcRs). IgG is the most abundant antibody in the blood and is further classified into four subtypes, IgG1, IgG2, IgG3, and IgG4, while IgA consists of the two subtypes IgA1 and IgA2. After activation, B cells can undergo class switch recombination and expression of antibody isotypes can change from IgM/IgD to IgA, IgE or IgG, or they can keep IgM expression. The Ag valency dictates the magnitude and composition of B cell responses, whereby high valency enables strong activation and effector differentiation as well as long-lasting B cell memory responses [5]. B cells that have bound to Ag can get activated in a T-cell independent or a T-cell dependent manner in the germinal centres to become plasma cells or memory B cells. Plasma cells migrate to the bone marrow and can reside there for a long time, while memory B cells circulate in the body and can get reactivated quickly on another encounter of the Ag.

To induce strong T cell responses, effector differentiation and T memory cells, Ag peptides or proteins alone are usually not very effective as vaccine since these are inefficiently taken up by antigen-presenting cells (APCs). The uptake efficiency of the Ag peptide or protein by APCs can be greatly increased, by either fusing an APC-targeting moiety e.g. by expressing an Ag fused to the crystallizable fragment (Fc) of an immunoglobulin (Ig) or when the Ag is coated with antibodies to form immune complexes (ICs). For example, when monocyte-derived dendritic cells were pulsed with polyclonal tetanus IgG ICs a stronger activation could be achieved as compared to the ‘naked’ tetanus toxin Ag [6]. Subsequent proteolytical cleavage via the lysosomal pathway (e.g. in case of phagocytosed Ag) or the cytoplasmic proteasomal pathway (e.g. pathogenic proteins produced in the cytosol) generates Ag-derived peptides that are loaded on the major histocompatibility complexes (MHC) type I or type II, while lysosomal derived peptides usually are loaded on MHC-II and peptides derived from the cytoplasmic proteasomal pathway are loaded on MHC-I. Antigenic peptides that are generated in lysosomes can however also be transferred into the cytoplasm and loaded on MHC-I complexes, a process that is referred to as cross-presentation and is most common in dendritic cells [7]. The MHC-I or MHC-II presented peptides can stimulate CD8+ or CD4+ T-cell responses, respectively. In addition to MHC-presented peptides, secondary activating signals are needed for efficient T-cell activation, including expression of T cell CD28 binding molecules on APCs (e.g. CD80, CD86) and the secretion of cytokines by the APC [7].

Fc-fusion proteins constitute pathogen-derived Ag fused to the Fc of an Ig. The aim of this strategy is to direct the uptake of the antigenic moiety into APCs via the interaction with an Fc receptor (FcR) on the APC. Several Fc-fusion proteins have been experimentally tested for vaccination and 13 Fc fusion proteins were approved by the European Union and/or the United States as of 2020 [8]. A recombinant vaccine of the Spike receptor binding domain (RBD) fused to Fc from IgG has been developed as vaccine against SARS-CoV-2 and is under testing in phase I/II human clinical trials [9]. Similarly, an oligomeric influenza haemaglutinine HA-Fc fusion protein induced epitope specific, neutralizing antibody responses in mice [10]. An IgG2a Fc fused to the Epstein-Barr virus (EBV) protein gp350 induced a potent neutralizing immune response in Balb/c mice [11]. A fusion protein of the Ebolavirus glycoprotein with Fc induced good humoral and cellular responses, including a robust CD8+ T cell response and protected mice from a lethal Ebolavirus infection [12]. A fusion protein consisting of HSV-2 gD protein fused to an IgG1 Fc fragment induced efficient mucosal and systemic, B and T cell immunity, including memory responses that were stable for at least 6 months [13]. All these experimental approaches demonstrated the suitability of recombinant Fc-fusion proteins for immunization against various viral pathogens.

Similar experimental approaches showed effects of Fc-fusion proteins also as possible antitumour vaccines. For example, an Xcl1-OVA-Fc fusion construct could be taken up efficiently by dendritic cells and stimulated potent H-2kb/OVA-specific T-cell responses that were able to control tumour progression in mice. The vaccine increased Ag-specific tumour-infiltrating CD8+ T-cells with elevated IFNg production and degranulation potential [14].

Likewise, approaches have been attempted to use Fc fusion proteins as vaccination strategy against some bacterial infections. For example, an IgG2a Fc-PspA fusion protein polarized alveolar macrophages (AM) towards an activated AM1 phenotype, induced Th1 cell polarization and activated conventional dendritic cells, suggesting good potential as a vaccine candidate against Streptococcus pneumoniae [15]. To induce immune responses against common fungal antigens, like chitin and beta-glucans, Dectin1-Fc(IgG2a), Dectin1-Fc(IgG2b) and wheat germ agglutinin (WGA)-Fc(IgG2a) fusion proteins have been examined for their potential to induce immune responses against several fungal pathogens and antifungal activity could be demonstrated against Histoplasma capsulatum, Cryptococcus neoformans, Candida albicans and Aspergillus fumigatus [16]. Finally, Fc fusion proteins have been tested as vaccine strategies against protozoan pathogens, such as Plasmodium falciparum causing malaria [17].

Immune complexes (ICs) are generated during the natural humoral immune response, when endogenously produced usually polyclonal antibodies bind polyvalently to the microbial target antigen(s) usually present at multiple copies on the outside of the intruding pathogen, forming large antibody-Ag complexes. The antibodies opsonize the microbial agent by binding with their Fab region to exposed epitopes in target antigens which can also lead to allosteric or structural Fab-mediated changes in the target antigen and can thereby cause the exposure of novel B-cell epitopes. Potentiating effects of ICs on the B-cell response have been attributed to the function of follicular dendritic cells (FDC) which can capture and retain intact ICs to serve as long-lasting Ag depots for B-cell stimulation [18, 19]. FDCs can be found in primary follicles and in germinal centres of secondary and tertiary lymphoid organs, such as spleen, lymph nodes and gut-associated lymphoid tissues where they can encounter Ag-specific B cells and help in the B cell affinity maturation by somatic hypermutation. These B cells can phagocytose the ICs presented by FDCs and can then stimulate CD4+ T-cells by MHC-II presentation of IC-derived Ag peptides, a process that feeds back promoting B-cell proliferation and finally antibody secretion.

Another key feature of ICs is that polyvalent binding of many antibody molecules (opsonization) covers the pathogen or tumour cell with Fc domains which increases the effector function through avidity binding of Fc to FcR on APCs. Interaction of ICs with FcR on APCs can lead to the uptake of the ICs via phagocytosis and the activation of the APC. Potentiating effects of ICs on antigen presentation and T-cell stimulation have long been known [20]. In addition, ICs may also suppress T-cell activation under certain circumstances including antibody-excess and polyclonality of antibodies [21]. When comparing an IC vaccine against Mycobacterium tuberculosis either in excess of Ag or of antibody, animals that were immunized with ICs in excess of Ag survived a lethal infection while animals of the antibody excess IC group showed some mortality [22].

Immune complex (IC) vaccination strategies are aiming to resemble naturally occurring complexes of target Ag(s) bound to antibodies to immunize against the Ig-opsonized Ag(s). Multivalent binding of IgGs to their target(s) within an IC thereby is important for the induction of a strong and durable humoral immune response. IC vaccination approaches have been experimentally tested against a variety of pathogens. Yeast-derived hepatitis B virus (HBV) surface antigen (HBsAg) was used together with antibodies derived from hyper-immunized donors to generate an IC vaccine against HBV. In a phase I clinical study with healthy adults, serum anti-HBV antibodies could be generated and interferon gamma (IFNg) and interleukin 2 (IL-2) levels could be efficiently increased, without any safety issues [23]. The subsequent phase IIa study with HBeAg-positive chronic hepatitis B patients demonstrated reduction of viral load in half of the treated patients and showed T-cell cytokine responses in some, indicating a therapeutic effect of this vaccine. In addition, dendritic cells from these patients derived from PBMCs demonstrated activation and maturation upon ex vivo stimulation with this IC vaccine [24]. Interestingly, a phase IIb trial demonstrated no statistically different effects between the immunized group and placebo in the relevant primary and secondary endpoints [25] and a subsequent phase III aimed to achieve better results by increasing the number of injections of IC vaccine from six (phase IIb) to twelve (phase III). Surprisingly, even worse outcomes were noted as compared to phase IIb, suggesting that merely by increasing the dose of IC vaccine does not necessarily enhance the immune response [26]. The stimulation of de novo neutralizing antibodies and protective CD8+ T cell responses was observed in different SIV/SHIV animal models when using polyclonal or broadly neutralizing antibody therapies and this was proposed to be a result of the formation of ICs build by infused antibody binding to circulating virions or Ag and uptake of these ICs by APCs followed by MHC-I cross-presentation [27-30]. Bursaplex® vaccine, an IC vaccine, has been approved to protect poultry from infectious bursal disease virus (IBDV). In addition, IC vaccine strategies have also been developed against other veterinary viral diseases induced by equine herpesvirus 1 as well as porcine parvovirus. An IC-vaccine using the IgG-opsonized sE protein of Tick-Borne Encephalitis virus induced similar antibody responses in mice as compared to the sE protein alone, however analysis of fine specificities of induced antibodies suggested difference, suggesting that sE-IgG ICs may have revealed novel B-cell epitopes by antibody-induced structural changes [31]. IC-vaccine strategies have also been explored against other major human pathogens including human immunodeficiency virus [32], as well as against bacterial infections [33]. Soluble ICs as well as dendritic cells loaded with ICs were also investigated as antitumour vaccines [34].

Intriguingly, while using ICs as vaccine may provide a reasonable way to induce protective immune responses, also possible adverse effects have been described that need to be considered in the vaccine design. ICs can dysregulate immune responses in chronic infectious diseases and have been shown to impact inflammatory responses in autoimmune diseases by prolonged engagement of FcRs. This can lead to aberrant signalling and sustained inflammatory responses, including elevated levels of pro-inflammatory cytokines, activated lymphocytes and finally exhausted immune cells. Circulating ICs have for example been suggested to impair the antibody-mediated clearance of opsonized target cells in chronic lymphocytic choriomeningitis virus (LCMV) infected mice. When anti-CD20 rituximab was injected in LCMV chronically infected mice, B cell clearance was substantially reduced due to the presence of ICs which competitively bound FcgRs and blocked their function in rituximab-induced B cell clearance [35]. Agonistic anti-CD40 antibodies were also compromised in FcgR-dependent activation of dendritic cells in these mice. The authors propose that in chronic viral infections FcgR-mediated effector functions are reduced due to circulating ICs. Intriguingly, for other chronic virus infection such as HBV or hepatitis C virus (HCV) infection circulating ICs also have been described [36], suggesting that the observed effects may be relevant also for other chronic viral infections, possibly also for Herpes Simplex Virus (HSV) infection. Indeed, in serum samples from patients with erythema multiforme, a skin reaction that can be triggered by HSV infection, increased levels of HSV ICs were detected [37], suggesting that HSV-ICs can form and may under chronic recurrent conditions exert adverse effects on FcR function.

The Fc moiety of IgGs confers the capability to exert effector functions, including antibody-dependent cellular cytotoxicity (ADCC), complement-dependent cytotoxicity (CDC) and antibody-dependent phagocytosis (ADCP).

In ADCC, antibodies that have bound their target activate effector cells, which are mainly natural killer (NK) cells but can also be macrophages, neutrophiles or eosinophiles. Activation leads to killing of the antibody-bound target cell. In CDC, complement protein C1q binds to Fc domains of IgG1s, IgG3s or IgMs that bound to their target Ag, which leads to a cascade of events and finally in the formation of the membrane attack complex and lysis of the target cells. The different IgG isotypes interact with distinct binding affinities with the different FcgR that exist on human cells. IgG1 binds with high affinity to FcgRI and exerts ADCP, binds with medium affinity to FcgRIIaH131 and with low affinity to FcgRIIaR131, both also mediating ADCP, binds with low affinity to FcgRIIb variants, relevant for immunosuppression and with medium and low affinity to FcgRIIIaV158 and FcgRIIIaF158, respectively, both mediating ADCC [38]. IgG1 has also the highest affinity to C1q to activate CDC. Antibodies of the IgG2 isotype do not interact with FcgRI, bind with medium and low affinities to FcgRIIaH131 and FcgRIIaR131, mediating myeloid-cell induced ADCC, do not interact with FcgRIIb variants and bind with low affinity to FcgRIIIaV158, but not to FcgRIIIaF158. Antibodies of the IgG3 isotype bind with high affinity to FcgRI exerting ADCP, with low affinity to FcgRIIa or FcgRIIb variants, and with medium affinity to FcgRIIIa variants [39]. Antibodies of the IgG4 isotype bind with high affinity to FcgRI and can mediate ADCP and cytokine release, do interact with low affinity with FcgRIIa variants inducing receptor clustering and interact with low affinity with FcgRIIb variants, supporting clearance of IC and immunosuppression, while no interaction with FcgRIIIa variants has been observed [38]. To date only IgG1, IgG2 and IgG4 therapeutic antibodies have been clinically approved. The formats are relatively similar and mostly differ in their hinge and CH2 regions. IgG4 for example has a unique S228 residue in the hinge region, that can induce interchangeable disulfide bond configurations. This can lead in vivo to shuffling and result in monovalent-bispecific antibodies through Fab-arm exchange, which can be prevented via engineering of S228P amino acid substitution. IgG2 can adopt structurally different disulfide isomers (IgG2A, IgG2B, IgG2A/B) and transformation between different isomers has been shown to impart FcR-independent superagonistic properties attributed to IgG2B [40]. While also being able to exert Fc effector functions through high affinity with FcgRI and medium affinity to FcgRIII variants, antibodies of the IgG3 isotype have long hinge regions and complex disulfide bonds, which confers a greater polymorphism that is thought to increase their immunogenicity, a reason that hampered the development of IgG3 for clinical application to date.

FcgRI, FcgRIIa and FcgRIIIa function as activating receptors with the signal transduction motif, immunoreceptor tyrosine-based activation motif (ITAM), located in the g subunit of FcgRI and FcgRIIIa, while FcgRIIa carries this motif in its cytoplasmic tail. In contrast, crosslinking of the inhibitory receptor FcgRIIb causes the phosphorylation of the immunoreceptor tyrosine-based inhibitory motif (ITIM) and transduction of inhibitory signals. FcgRI is a high affinity receptor for IgG1 IgG3 and IgG4 and is mainly expressed on activated neutrophiles and myeloid cells including dendritic cells and monocytes/macrophages. FcgRI has high affinity for monomeric IgG1 and IgG3, hence it is thought that most of FcgRI molecules are occupied with free monomeric IgG1 due to high serum IgG1 concentrations of ^{˜}15 mg/ml. In the presence of proinflammatory cytokines interleukin 3 (IL3), tumor necrosis factor-a (TNFa) or interferon g (IFNg) clustering of FcgRI was induced resulting in a better uptake of ICs and demonstrating that nanoscale reorganisation of FcgRI could enhance FcgRI-mediated effector functions [41]. In addition to FcgRI, FcgRIIa is the main mediator for ADCP. While FcgRIIa does only have low and medium affinities for monomeric IgG1 and IgG3, respectively, it strongly binds to ICs, similarly to FcgRI, resulting in ADCP. The primary receptor for ADCC-induced cell killing in humans is FcgRIIIa, which is primarily expressed on natural killer (NK) cells and macrophages. In contrast, FcgRIIb is an inhibitory receptor expressed on immune cells, including B cells, dendritic cells, monocytes/macrophages, mast cells and basophils, as well as on liver sinusoidal endothelial cells (LSEC), where it also plays a critical role in clearing ICs. Knock-out of FcgRIIb in mice severely reduced clearance of blood-borne ICs [42]. Indeed, blood-borne HIV-1 ICs were demonstrated to be cleared by LSECs via FcgRIIb-mediated endocytosis and transport to the lysosome, showing that viral ICs can also be cleared through FcgRIIb [43]. While FcgRIIb-mediated phagocytosis of ICs in LSEC can promote tolerogenic responses (by e.g. induction of Tregs), LSECs can also shift towards immunogenic responses, depending on the cytokine milieu. To which degree FcgRIIb expressed on other immune cells may also contribute to IC clearance is largely unknown. However, knock-out studies suggest that FcgRIIb negatively impacts the phagocytosis of immune complexes in macrophages [44]. Mutation of IgG1 at glycosylation site N297 leads to reduced effector functions, due to reduced binding to low affinity FcgRs. In contrast, in the context of increased avidity (e.g. by multiple mAb copies within an IC), N297-mutated IgG1 can still bind to FcgRI, however the efficiency depends on the levels of present free monomeric IgGs that compete with the binding to FcgRI. In the presence of high levels of monomeric IgG1, FcgRI binding to IgG1-N297A ICs was strongly reduced [45].

IgGs do not only interact with FcgR but also with non-classical FcRs, including the neonatal FcRn and the cytosolic tripartite motif protein 21 (TRIM21).

The neonatal FcRn is an MHC-I-related receptor that can interact with IgGs in form of a heterodimer consisting of the heavy a-chain and the b2-microglobulin. FcRn is involved in transport e.g. by mediating transcytosis, distribution and persistence of IgG antibodies. FcRn interaction with IgG is dependent on several histidine residues that are located on the exposed loops at the CH2-CH3 domain interface (e.g. His435), which interact with acidic residues of FcRn. This interaction confers the characteristic high affinity at acidic pH and relatively weak affinity at pH 7.3-7.4, that is present for most IgG subclasses. Hence FcRn is important to maintain serum IgG levels by recycling. Certain human IgG3 allotypes have Arg435 and hence have a relatively high affinity binding to FcRn at pH6 as well as at neutral pH due to the lack of protonation/deprotonation cycles at the different pHs. In addition, FcRn participates in the transport of ICs for MHC-presentation in hematopoietic cells. ICs are usually taken up by FcgR-induced phagocytosis and in early endosomes once pH becomes acidic transferred to FcRn. FcRn then supports delivery of multimeric IgG-Ag complexes but not monomeric IgG-Ag complexes towards proteolytic degradation and MHC-I cross-presentation or MHC-II presentation stimulating T-cell responses [46]. Indeed, FcRn knock-out mice demonstrated reduced CD4+ expansion after IC stimulation, supporting a function of FcRn in MHC-II Ag-presentation for T-cell stimulation [47]. Supporting this model, FcRn was found in a ternary complex with FcgRIIa and IC and it was shown that FcRn blockade with an antibody reduced IC-induced inflammation in a rheumatoid arthritis model, demonstrating that FcRn is involved in pro-inflammatory IC-induced immune responses [48]. FcRn is also important for the uptake of IgG-opsonized pathogens by neutrophiles, the most abundant phagocytic cell of the body. In addition, FcRn overexpression in mice was also shown to increase T cell responses [49]. Cross-presentation of Ag-derived peptides after IC phagocytosis on MHC-I is thought to be dependent on APC cell type as well as the nature of the Ag bound within the IC and the uptake route and can occur via a vacuolar or a cytosolic pathway. Not only the Fc domain of IgG is important for FcRn binding, but also the variable Fab region can impact FcRn binding and subsequent effects, suggesting a more complex interplay [50].

The dimeric cytosolic non-classical Fc-receptor TRIM21 can bind via its C-terminal PRYSPRY domains to dimeric Fc present in IgG and has a ubiquitin-ligase enzymatic domain that allows polyubiquitination of IgG-bound target protein and subsequent proteasomal degradation. Complexes of IgG bound to viral Ag can be taken up into the cytosol through largely unknown mechanisms and TRIM21 binding to IgG and ubiquitination will induce an antiviral state in the cell, a process that is tightly regulated through autoubiquitination of TRIM21 as well as phosphorylation. Like FcRn, TRIM21 binds to the CH2-CH3 region of the Fc domain. As a consequence of TRIM21 binding the IgG-Ag complex is firstly monoubiquitinated, then polyubiquitinated via K63- and later branched K48-linked ubiquitin chains and directed to the proteasome, where the deubiquitinating enzyme Pohl liberates the ubiquitin chains en bloc and induces NFkB, AP1, IRF3, IRF5 and IRF 7 signalling which causes the production of pro-inflammatory cytokines. Evidence exist that TRIM21-mediated proteolytic degradation of Adenoviral vectors may impact MHC-I presentation and induction of T-cell responses [51]. Induction of proinflammatory cytokines through TRIM21-mediated ubiquitination has largely been studied on non-enveloped Adenoviral vector systems and bacteria, however, non-neutralizing antibodies binding to N-protein from LCMV, an enveloped virus, can also be bound by TRIM21 and also change N-specific T cell responses to confer protection from infection [52].

In addition to the active immunization strategies using Fc-fusion proteins or ICs as described above, during which the host immune response is sought to be induced by the exposure of the host to foreign signatures or structures, in passive immunization, antibodies are given as prophylaxis or as therapy against microbial agents with the aim to neutralize their pathogenic activity and to give a short-term protection from the pathogen and/or the associated disease. This type of immunity can protect against measles, mumps, whooping cough, poliomyelitis, rabies, rubella, tetanus, chickenpox and herpes zoster virus infection. The administration of antibodies is also used as a treatment of symptoms associated with snake or spider bites and also as immunosuppressant (Rho-GAM, antilymphocyte serum). Passive immunization is often ineffective, since the duration of immunity is brief and can largely vary from person to person. Long-term protections after passive antibody therapies have not been described so far.

The first monoclonal antibody (mAb) muromonab-CD3 was a murine mAb against CD3 that functioned as was approved by the FDA in 1986 and in 2021 the 100^{th} mAb was approved of which approximately one third have been developed for the treatment of cancer. Monoclonal antibody therapies have been developed and clinically approved by the FDA for the treatment of several different indications, including infectious diseases, such as SARS-CoV-2 infection/COVID-19 (Bamlanivimab/Etesevimab; Casirivimab/Imdevimab; Sotrovimab, all targeting Spike protein), HIV infection (Ibalizumab, targeting CD4), respiratory syncytial virus (RSV) infection (Palivizumab, targeting the A antigenic site of the RSV F protein), anthrax (Obiltoxaximab, Raxibacumab), Clostridium difficile infection (Bezlotoxumab), diverse types of cancers, such as bladder cancer (Durvalumab; Atezolizumab, both targeting PD-L1), hairy cell leukemia (Moxetumomab pasudodox, targeting CD22), diffuse large B cell lymphoma (Polatuzumab vedotin, targeting CD79b), cutaneous squamous cell carcinoma (Cemiplimab, targeting PD-1), acute myeloid leukaemia (Gemtuzumab ozogamicin, targeting CD33), Merkel cell carcinoma (Avelumab, targeting PD-L1), acute lymphoblastic leukaemia (Inotuzumab ozogamicin, targeting CD22; Blinatumomab, bispecific targeting CD19 and CD3), soft tissue sarcoma (Olaratumab, targeting PDGFRa), multiple myeloma (Daratumumab, targeting CD38; Elotuzumab, targeting SLAMF7), neuroblastoma (Dinutuximab, targeting GD2), non-small cell lung cancer (Necitumumab, targeting EGFR), melanoma (Nivolumab; Pembrolizumab, both targeting PD-1), gastric cancer (Ramucirumab, targeting VEGFR2), chronic lymphocytic leukaemia (Obinutuzumab; Ofatumumab, both targeting CD20), breast cancer (Pertuzumab; Trastuzumab; Trastuzumab emtansine, all targeting HER2), Hodgkin lymphoma (Brentuximab vedotin, targeting CD30), metastatic melanoma (Ipilimumab, targeting CTLA-4), colorectal cancer (Panitumumab; Cetuximab, both targeting EGFR; Bevacizumab, targeting VEGF-A), Non-Hodgkin lymphoma (Ibritumomab tiuxetan; Rituximab, both targeting CD20), chronic myeloid leukaemia (Alemtuzumab, targeting CD52), as well as other diseases including for example asthma (Omalizumab, targeting IgE), rheumatoid arthritis (Adalimumab, targeting TNFa), Crohn's disease (Infliximab, targeting TNFa), or transplant rejection (Daclizumab, targeting IL2). Besides approved mAb therapies, as of December 2019, worldwide >570 therapeutic mAbs were under investigation in clinical trials.

In light of the prior art, there is a need to provide further means for the effective treatment or prevention of infectious diseases, in particular with respect to persistent or long-term effects.

The present invention is, in part, based on the surprising observation that an antibody (having a certain specificity towards an antigen) is exclusively capable of inducing antibody dependent cellular phagocytosis (ADCP) without eliciting antibody-dependent cellular cytotoxicity (ADCC) and/or complement-dependent cytotoxicity (CDC).

This surprising finding of the present invention leads to the unexpected possibility that antibodies can be used as a vaccine to actively immunize a subject against an infectious disease or a malignant disease which, in particular, is linked to the beneficial effect that a persistent and long-term immunity is generated.

Thus, in a nutshell, based on the observed effect, the present invention relates, in its broadest term, to the therapeutic use of antibodies or antibody-derived molecules, e.g., Fc fusion proteins or a combination thereof as a strategy to actively immunize individuals against the targeted antigen through the induction of de novo endogenous T- and/or B-cell immune responses.

According to the present invention, shifting Fc-effector functions towards ADCP while avoiding ADCC and CDC induces long-lasting responses and enhances de novo responses also against epitopes that are revealed through the binding of the (monoclonal) antibody.

The mode of action of described monoclonal antibodies is to bind to specific targets thereby inactivating or activating the target. This can be merely by masking a site through mAb-binding but can also involve the induction of Fc-mediated ADCC, or CDC.

Therapeutic monoclonal antibodies that do not induce CDC or ADCC, but exclusively induce ADCP have not been described in the art according to the best of our knowledge.

The present invention, thus, is, in part, based on the proposal, that modification of monoclonal antibodies towards activating ADCP while at the same time avoiding CDC and ADCC leads to a long-lasting active immunization.

This is, in fact, surprising in light of the prior art discussed above and based on the experimental evidence of the Examples.

Indeed, the present invention surprisingly demonstrates that a therapeutic anti-HSV IgG1 has long-lasting protective effects, including evidence from a clinical study. Without being bound to theory, based on this surprising observation of the present invention, a long-lasting vaccine-like effect can be induced by the formation of ICs through monoclonal antibody target binding, followed by ADCP, thereby inducing proteolytic processing that leads to the enhanced presentation of target peptides on MHC-I via cross-presentation or MHC-II for the induction of de novo CD8 and CD4 T-cell responses, respectively. This leads to the rationale of the present invention.

The absence of ADCC and CDC is thereby an important feature to prevent unwanted immunological side effects and imbalances, such as tolerogenic responses due to the killing of target cells. The experimental results of the present invention provide evidence that shifting the Fc-function of antibody-opsonized pathogens towards an exclusive ADCP-inducing functionality and avoiding ADCC and CDC will generate long-lasting immunological vaccine-like effects.

ADCC and CDC inevitably lead to the destruction of cells or pathogens by proteolytic digestion, e.g., through the serin protease granzyme B that is secreted by natural killer cells or cytotoxic T cells. In contrast, endocytosed Ag-ICs are mainly digested by lysosomal proteases, including cysteine proteases Cathepsins B, K, H, L and S and aspartyl protease Cathepsin D. We propose that exclusively targeting ADCP and avoiding CDC and ADCC will result in a more potent and possibly broader T-cell peptide presentation on MHC complexes. Changes in T-cell responses after monoclonal therapy have been suggested before, however, the findings of the present invention reveal a new pathway that enhances de novo T-cell and B-cell responses through the combination of the specific enhancement of ADCP, the induction of a specific cytokine response and at the same time the eradication of ADCC and CDC functions in therapeutic antibodies that has never been proposed before.

In view of the prior art, the technical problem underlying the present invention is the provision of further means and methods for the treatment and/or prevention of (recurrent) infectious or malignant diseases, in particular, with respect to long-term or persistent effects. The advantage of these further means and methods for the treatment and/or prevention provided by the present invention is, inter alia, that individuals/subjects that have to be treated would simultaneously acquire a long term immunity against said infectious or malignant diseases in terms of a vaccination.

The technical problem is solved by provision of the embodiments characterized in the claims.

As mentioned above, it has surprisingly been demonstrated in the appended examples that an antibody (having a certain specificity towards an antigen) is exclusively capable of inducing antibody dependent cellular phagocytosis (ADCP) without eliciting antibody-dependent cellular cytotoxicity (ADCC) and/or complement-dependent cytotoxicity (CDC).

This finding is in particular surprising and unexpected in light of the prior art discussed above. As mentioned, this surprising finding of the present invention leads to the unexpected possibility that antibodies can be used as a vaccine to actively immunize a subject against an infectious disease or a malignant disease which, in particular, is linked to the beneficial effect that a persistent and long-term immunity is generated. However, the present invention is not limited to the concept of using an antibody against an antigen correlated with said infectious disease or malignant disease as a vaccine for use in actively immunising a subject against an infectious disease or a malignant disease. Based on this rationale, it is also envisaged that an Fc-containing fusion protein comprising an Fc part of an antibody fused to an antigen correlated with said infectious disease or malignant disease can be used as a vaccine for use in actively immunising a subject against an infectious disease or a malignant disease.

Thus, in a first aspect, the present invention relates to a vaccine for use in actively immunising a subject against an infectious disease or a malignant disease, said vaccine comprising:
(a) an antibody against an antigen correlated with said infectious disease or malignant disease; or
(b) an Fc-containing fusion protein comprising an Fc part of an antibody fused to an antigen correlated with said infectious disease or malignant disease, wherein said Fc part is capable of binding a receptor present on or in an antigen presenting cell (APC) selected from the group consisting of:
   Type I: activatory FcγRI, FcγRIIa, FcγRIIc, FcγRIIIa, FcγRIIIb, and inhibitory FcγRIIb; and
   Type II: neonatal FcR (FcRn) and cytosolic TRIM21.

Moreover, in a second aspect, which is related to the above first aspect, the present invention relates to a vaccine for use in actively immunizing a subject against an infectious disease, wherein said infectious disease is an HSV-associated disease; and
(a) wherein said antibody is an anti-HSV antibody and said subject suffers from an acute HSV-associated disease, preferably an acute HSV infection; or
(b) wherein in said Fc-containing fusion protein comprising an Fc part of an antibody fused to an antigen, the antigen correlates with an HSV-associated disease.

The second aspect is described in more detail further below.

In the following, the first aspect, is described in more detail.

Thus, the present invention generally relates to a vaccine.

A vaccine commonly refers to an “immunogenic composition” that comprises at least one agent that resembles a disease-causing virus or microorganism. Thus, a vaccine is generally a biological preparation that normally provides active acquired immunity to a particular infectious disease. A vaccine is often made from weakened or killed forms of the microbe, its toxins, or one of its surface proteins. The agent stimulates the body's immune system to recognize the agent as a threat, destroy it, and to further recognize and destroy any of the microorganisms or viruses associated with that agent that it may encounter in the future.

A vaccine or an immunogenic portion thereof commonly elicits an immunological response (cellular or antibody-mediated immune response) in the host to the composition.

The term “vaccine” as used herein in terms of the present invention refers, however, to a composition which does not itself comprise at least one agent that resembles a disease-causing virus or microorganism. Instead, the vaccine comprises (a) an antibody against an antigen correlated with said infectious disease or malignant disease; or (b) an Fc-containing fusion protein comprising an Fc part of an antibody fused to an antigen correlated with said infectious disease or malignant disease, wherein said Fc part is capable of binding a receptor present on or in an antigen presenting cell (APC) selected from the group consisting of: Type I: activatory FcγRI, FcγRIIa, FcγRIIc, FcγRIIIa, FcγRIIIb, and inhibitory FcγRIIb; and Type II: neonatal FcR (FcRn) and cytosolic TRIM21.

As outlined above and below, it is proposed that said vaccine in terms of the present invention elicits an immunological response (i.e., a cellular or antibody-mediated immune response) in an animal or a subject by eliciting antibody dependent cellular phagocytosis (ADCP), more specifically, by mediating an Fc-based effector function by an antibody dependent cellular phagocytosis (ADCP). In other words, it is proposed that said vaccine in terms of the present invention elicits an immunological response by eliciting antibody dependent cellular phagocytosis (ADCP) via interaction with any of the above FcRs, and/or by eliciting antibody dependent cellular phagocytosis (ADCP) leading to the production of cytokines by the antigen presenting cell (APC). It is also proposed that said vaccine in terms of the present invention elicits an immunological response by eliciting antibody dependent cellular phagocytosis (ADCP) leading to the generation and antigen presenting cell (APC) presentation of de novo HSV-derived peptides; and/or eliciting antibody dependent cellular phagocytosis (ADCP) leading to the activation of a T-cell (and/or B-cell) immune response against de novo HSV-derived peptides; and/or by eliciting antibody dependent cellular phagocytosis (ADCP) leading to the generation and antigen presenting cell (APC) presentation of HSV-derived peptides.

A vaccine may additionally comprise further components typical to pharmaceutical compositions as defined below.

As used herein, the terms “vaccine” and “vaccine composition” are used interchangeably and in particular refer to a composition that will elicit a protective immune response in a subject that has been exposed to the composition in terms of the present invention as outlined above and below.

The vaccine of the present invention is for use in actively immunising a subject against an infectious disease or a malignant disease.

“Active immunization” in terms of the present invention refers to the induction of immunity after exposure to the above-defined vaccine, ultimately leading to eliciting an immunological response (cellular or antibody-mediated immune response). Generally, active immunization aims to ensure that a sufficient supply of antibodies or T- and B-cells that react against a potential infectious agent or toxin are present in the body, preferably, before infection occurs or the toxin is encountered.

Active immunization in terms of the present invention is opposed to “passive immunization”. Passive immunity is commonly understood as the transfer of active humoral immunity of ready-made antibodies. Passive immunity can occur naturally, when maternal antibodies are transferred to the fetus through the placenta, and it can also be induced artificially, when high levels of antibodies specific to a pathogen or toxin (obtained from humans, horses, or other animals) are transferred to non-immune persons through blood products that contain antibodies, such as in immunoglobulin therapy or antiserum therapy.

The vaccine of the present invention (comprising (a) an antibody against an antigen correlated with said infectious disease or malignant disease; or (b) an Fc-containing fusion protein comprising an Fc part of an antibody fused to an antigen correlated with said infectious disease or malignant disease, wherein said Fc part is capable of binding a receptor present on or in an antigen presenting cell (APC) selected from the group consisting of: Type I: activatory FcγRI, FcγRIIa, FcγRIIc, FcγRIIIa, FcγRIIIb, and inhibitory FcγRIIb; and Type II: neonatal FcR (FcRn) and cytosolic TRIM21), although it comprises an antibody component and an Fc part of an antibody component, respectively, is not used in passively immunising a subject.

In contrast, against the common understanding, the vaccine of the present invention comprising the above antibody component and an Fc part of an antibody component, respectively, is used in actively immunising a subject.

Active immunization can be monitored by periodic assessment by the skilled person by applying routine methods. Without being bound to theory, active immunization can, e.g., be monitored by analysing antibody levels against the antigen used for vaccination in the serum of vaccinated individuals. Serologic switch from seronegative to seropositive for immunoglobulins indicates that the vaccine actively induced a humoral immune response. Several methods have been established to test for seroconversion. The best known is Enzyme-linked immunosorbent assay (ELISA). Antigen is coated on a plate and serum is added. If seroconversion, the antibodies in the serum will bind to the antigen on the plate and can be detected by a secondary antibody usually coupled to an enzyme. In contrast to passive immunization, in active immunization these antibodies are derived from the vaccinated person. Addition of a substrate induces a colour switch by this enzyme and this can be measured using a photometer. Alternative assays include immunoblotting, electroluminescence based ELISAs, measurements using plasmon surface resonance or bio-layer interferometry, immunofluorescence based flow cytometry or microscopy.

In addition to this B-cell-mediated immune response active immunization is also inducing T-cell responses towards peptides derived from the vaccine antigen. T-cell responses towards the vaccine antigen can be for example measured by incubation of primary blood mononuclear cells (PBMCs) with the vaccine antigen and measurement of interleukin-2 (IL-2) or interferon gamma (IFNγ) secretion by activated T-cells using e.g. an ELISpot assay, cytokine ELISAs, or flow-cytometric assays in which released cytokines are bound by fluorescent beads. Active immunization is also seen by the changes in the T-cell receptor (TCR) and B-cell receptor (BCR) repertoire. Active immunization will induce clonal expansion of B-cells and T-cells that are directed against the vaccine antigen and hence when the entire repertoire is sequenced individual clones will become more prominent [55].

The vaccine of the present invention is for use in actively immunising a subject against an infectious disease or a malignant disease.

The term “infectious disease” is commonly known in the art and collectively refers in terms of the present invention to disorders caused by organisms such as bacteria, viruses, fungi or parasites. Many organisms live in and on our bodies. They're normally harmless or even helpful. But under certain conditions, some organisms may cause disease. An infection is commonly understood as the invasion of an organism's body or body tissues by disease-causing agents (like, e.g., bacteria, viruses, fungi or parasites), their multiplication or proliferation, and the reaction of the host's/subject's tissues to the infectious agents and the toxins they produce. An infectious disease, also known as a transmissible disease or communicable disease, is commonly referred to an illness resulting from an infection. As mentioned, infections can be caused by a wide range of pathogens, most prominently bacteria and viruses. Hosts/subjects can fight infections using their immune system. Subjects/hosts in terms of the present invention are preferrably mammalian hosts/subjects, more preferably human hosts/subjects which are known to react to infections with an innate response, often involving inflammation, followed by an adaptive response.

In preferred embodiments, the infectious disease is caused by infectious agents (often referred to as “pathogens” like, e.g., bacteria, viruses, fungi or parasites) are selected from the group consisting of:
Bacteria (preferably, Mycobacterium tuberculosis, Staphylococcus aureus, Escherichia coli, Clostridium botulinum, and Salmonella spp.);
Viruses and/or related agents such as viroids (preferably, HIV, Rhinovirus, Lyssaviruses such as Rabies virus, Ebolavirus and Severe acute respiratory syndrome coronavirus 2);
Fungi, further subclassified into:
   Ascomycota, including yeasts such as Candida (the most common fungal infection), filamentous fungi such as Aspergillus, Pneumocystis species, and dermatophytes, a group of organisms causing infection of skin and other superficial structures in humans. Basidiomycota, including the human-pathogenic genus Cryptococcus.
Prions
Parasites, which are usually divided into:

Unicellular organisms (e.g. malaria, Toxoplasma, Babesia)

Macroparasites (worms or helminths), nematodes (e.g., parasitic roundworms, and pinworms, tapeworms (cestodes), flukes (trematodes, e.g., schistosomes);

Arthropods (e.g., ticks, mites, fleas, and lice), can also cause human disease, which conceptually are similar to infections, but invasion of a human or animal body by these macroparasites is usually termed infestation.

In a more preferred embodiment, the vaccine defined above is for use in actively immunizing a subject against an infectious disease which is selected from the group consisting of fungal infections, bacterial infections, protozoan infections and viral infections.

In an even more preferred embodiment, said fungal infection is an infection by a Candida strain.

In another even more preferred embodiment, said bacterial infection is an infection by a Pseudomonas strain.

In another even more preferred embodiment, said protozoan infection is an infection by a malaria strains.

In another even more preferred embodiment, said viral infection is a HSV-associated disease (e.g., an HSV-associated disease caused by HSV-1- and/or HSV-2).

In line with the rationale of the present invention, the present invention is not particularly limited to a certain “infectious disease”. It is understood by the skilled person that the vaccine of the present invention can be tailored to any “infectious disease”, more specifically tailored to a corresponding “antigen correlated with said infectious disease”.

The term “malignant disease” is commonly known in the art and is understood, in its broadest sense, as the tendency of a medical condition to become progressively worse.

Malignancy is most familiar as a characterization of cancer. A malignant tumor contrasts with a non-cancerous benign tumor in that a malignancy is not self-limited in its growth, is capable of invading into adjacent tissues, and may be capable of spreading to distant tissues. A benign tumor has none of those properties.

Malignancy in cancers is characterized by anaplasia, invasiveness, and metastasis. Malignant tumors are also characterized by genome instability, so that cancers, as assessed by whole genome sequencing, frequently have between 10,000 and 100,000 mutations in their entire genomes. Cancers usually show tumor heterogeneity, containing multiple subclones. They also frequently have reduced expression of DNA repair enzymes due to epigenetic methylation of DNA repair genes or altered microRNAs that control DNA repair gene expression.

In a preferred embodiment, the malignant disease is a tumor, more preferably a cancer.

The term “cancer” refers to a group of diseases that involve abnormal increases in the number of cells, with the potential to invade or spread to other parts of the body. There are over 100 different known cancers that affect humans.

Cancers are often described by the body part that they originated in. However, some body parts contain multiple types of tissue, so for greater precision, cancers are additionally classified by the type of cell that the tumor cells originated from.

Accordingly, the vaccine for use in immunizing a subject against a malignant disease in terms of the present invention is a vaccine, wherein the malignant disease, more particularly, the cancer, is selected from the group consisting of:
Carcinoma. Carcinoma are cancers derived from epithelial cells. This group includes many of the most common cancers that occur in older adults. Nearly all cancers developing in the breast, prostate, lung, pancreas, and colon are carcinomas.
Sarcoma: Sarcoma are cancers arising from connective tissue (i.e., e.g., bone, cartilage, fat, nerve), each of which develop from cells originating in mesenchymal cells outside of the bone marrow.
Lymphoma and leukemia: These two classes of cancer arise from immature cells that originate in the bone marrow, and are intended to fully differentiate and mature into normal components of the immune system and the blood, respectively. A preferred type of leukemia is acute lymphoblastic leukemia. This type of leukemia is the most common type of cancer in children, accounting for approximately 30% of cases. A preferred type of lymphoma is diffuse large B cell lymphoma, which is the most common form accounting for approximately 30% of Non-Hodgkin lymphomas.
Germ cell tumor: Germ cell tumors are cancers derived from pluripotent cells, most often presenting in the testicle or the ovary (seminoma and dysgerminoma, respectively).
Blastoma: Blastoma are cancers derived from immature “precursor” cells or embryonic tissue. Blastomas are generally more common in children. Preferred blastomas are neuroblastoma, retinoblastoma, nephroblastoma, hepatoblastoma, medulloblastoma.

In more preferred embodiments, the vaccine for use in immunizing a subject against a malignant disease in terms of the present invention is a vaccine, wherein the malignant disease, more particularly, the cancer, is selected from the group consisting of:
bone and muscle sarcoma, cancer of the brain and/or cancer of the nervous system, cancer of the breast, cancer of the endocrine system cancer of the eye, gastrointestinal cancer, genitourinary or gynecologic cancer, head and neck cancer, hematopoietic cancer, cancer of the skin, thoracic and respiratory cancer, HIV/AIDS related cancer and unsorted cancers.

### Bone and Muscle Sarcoma.

In even more preferred embodiments, the Bone and muscle sarcoma is selected from the group consisting of: Chondrosarcoma, Ewing's sarcoma, Malignant fibrous histiocytoma of bone/osteosarcoma, Osteosarcoma, Rhabdomyosarcoma, Leiomyosarcoma and Myxosarcoma.

Cancer of the Brain and/or Cancer of the Nervous System.

In even more preferred embodiments, the cancer of the brain and/or the cancer of the nervous system is selected from the group consisting of: Astrocytoma, Brainstem glioma, Pilocytic astrocytoma, Ependymoma, Primitive neuroectodermal tumor, Cerebellar astrocytoma, Cerebral astrocytoma, Glioblastoma, Glioma, Medulloblastoma, Neuroblastoma, Oligodendroglioma, Pineal astrocytoma, Pituitary adenoma, and Visual pathway and hypothalamic glioma.

### Cancer of the Breast.

In even more preferred embodiments, the cancer of the breast (breast cancer) is selected from the group consisting of Inflammatory breast cancer, Invasive lobular carcinoma, Tubular carcinoma, Invasive cribriform carcinoma, Medullary carcinoma, Male breast cancer and Phyllodes tumor.

### Cancer of the Endocrine System.

In even more preferred embodiments, the cancer of the endocrine system is selected from the group consisting of: Adrenocortical carcinoma, Islet cell carcinoma (also termed endocrine pancreas), Multiple endocrine neoplasia syndrome, Parathyroid cancer, Pheochromocytoma, Thyroid cancer, and Merkel cell carcinoma.

### Cancer of the Eye.

In even more preferred embodiments, the cancer of the eye is selected from the group consisting of: Uveal melanoma, Retinoblastoma, and Optic nerve glioma.

### Gastrointestinal Cancer.

In even more preferred embodiments, the gastrointestinal cancer is selected from the group consisting of: Anal cancer, Appendix cancer, Cholangiocarcinoma, Carcinoid tumor, gastrointestinal, Colon cancer, Extrahepatic bile duct cancer, Gallbladder cancer, Gastric (stomach) cancer, Gastrointestinal carcinoid tumor, Gastrointestinal stromal tumor (GIST), Hepatocellular cancer, Pancreatic cancer (islet cell), Rectal cancer, and Small intestine cancer.

### Genitourinary or Gynecologic Cancer.

In even more preferred embodiments, the genitourinary or gynecologic cancer is selected from the group consisting of: Bladder cancer, Cervical cancer, Endometrial cancer, Extragonadal germ cell tumor, Ovarian cancer, Ovarian epithelial cancer (surface epithelial-stromal tumor), Ovarian germ cell tumor, Penile cancer, Kidney cancer, Renal cell carcinoma, Renal pelvis and ureter (transitional cell cancer), Prostate cancer, Testicular cancer, Gestational trophoblastic tumor, Ureter and renal pelvis (transitional cell cancer), Urethral cancer, Uterine sarcoma, Vaginal cancer, Vulvar cancer, and Wilms tumor (nephroblastoma).

### Head and Neck Cancer.

In even more preferred embodiments, the head and neck cancer is selected from the group consisting of: Esophageal cancer, Head and neck cancer, Nasopharyngeal carcinoma, Oral cancer, Oropharyngeal cancer, Paranasal sinus and nasal cavity cancer, Pharyngeal cancer, Salivary gland cancer and Hypopharyngeal cancer.

### Hematopoietic Cancer.

In even more preferred embodiments, the hematopoietic cancer is selected from the group consisting of: Acute biphenotypic leukemia, Acute eosinophilic leukemia, Acute lymphoblastic leukemia, Acute myeloid leukemia, Acute myeloid dendritic cell leukemia, AIDS-related lymphoma, Anaplastic large cell lymphoma, Angioimmunoblastic T-cell lymphoma, B-cell prolymphocytic leukemia, Burkitt's lymphoma, Chronic lymphocytic leukemia, Chronic myelogenous leukemia, Cutaneous T-cell lymphoma, Diffuse large B-cell lymphoma, Follicular lymphoma, Hairy cell leukemia, Hepatosplenic T-cell lymphoma, Hodgkin's lymphoma, Intravascular large B-cell lymphoma, Large granular lymphocytic leukemia, Lymphoplasmacytic lymphoma, Lymphomatoid granulomatosis, Mantle cell lymphoma, Marginal zone B-cell lymphoma, Mast cell leukemia, Mediastinal large B cell lymphoma, Multiple myeloma/plasma cell neoplasm, Myelodysplastic syndromes, Mucosa-associated lymphoid tissue lymphoma, Mycosis fungoides, Nodal marginal zone B cell lymphoma, Non-Hodgkin lymphoma, Precursor B lymphoblastic leukemia, Primary central nervous system lymphoma, Primary cutaneous follicular lymphoma, Primary cutaneous immunocytoma, Primary effusion lymphoma, Plasmablastic lymphoma, Sezary syndrome, Splenic marginal zone lymphoma, and T-cell prolymphocytic leukemia.

### Cancer of the Skin.

In even more preferred embodiments, the cancer of the skin is selected from the group consisting of: Basal cell carcinoma, Squamous cell carcinoma, Squamous cell skin cancer, Skin adnexal tumors (e.g. sebaceous carcinoma), Melanoma, Merkel cell carcinoma, Keratoacanthoma, Sarcomas of primary cutaneous origin (e.g. dermatofibrosarcoma protuberans), and Lymphomas of primary cutaneous origin (e.g. mycosis fungoides).

### Thoracic and Respiratory Cancer.

In even more preferred embodiments, the thoracic and respiratory cancer is selected from the group consisting of: Adenocarcinoma of the lung, Bronchial adenomas/carcinoids, Small cell lung cancer, Mesothelioma, Non-small cell lung cancer, Non-small cell lung carcinoma, Pleuropulmonary blastoma, Laryngeal cancer, Thymoma and thymic carcinoma, and Squamous-cell carcinoma of the lung.

### HIV/AIDS Related Cancer.

In even more preferred embodiments, the HIV/AIDS related cancer is Kaposi sarcoma.

### Unsorted Cancers.

There are also cancers known which are, however, not (yet) classified in any of the above groups of cancers. Corresponding cancers are collectively referred to as “unsorted cancers”. In more preferred embodiments, the unsorted cancer is selected from the group consisting of: Epithelioid hemangioendothelioma (EHE), Desmoplastic small round cell tumor and Liposarcoma.

In a more preferred embodiment, the vaccine defined above is for use in actively immunizing a subject against a malignant disease which is selected from solid tumors and malignant diseases of the blood/haematooncologic diseases.

A solid tumor is commonly referred to a tumor that does not contain any liquid or cysts.

Two major types of solid tumors in terms of the present invention are sarcomas and carcinomas.

Sarcomas are tumors in a blood vessel, bone, fat tissue, ligament, lymph vessel, muscle or tendon.

Preferred sarcomas are already outlined above and include, in particular embodiments, Ewing sarcoma and osteosarcoma, which are bone cancer sarcomas. Rhabdomyosarcoma, which is a soft tissue sarcoma found in muscles.

Carcinomas are tumors that form in epithelial cells. Epithelial cells are found in the skin, glands and the linings of organs. Those organs includes the bladder, ureters and part of the kidneys. Preferred carcinomas are already outlined above and include, in a particular embodiment, adrenocortical carcinoma. This is when a tumor develops in one or both adrenal glands, located above each kidney.

Diseases of the blood/haematooncologic diseases are already defined above.

As mentioned above, in line with the rationale of the present invention, the present invention is not particularly limited to a certain “infectious disease” or “malignant disease”. It is understood by the skilled person that the vaccine of the present invention can be tailored to any “infectious disease” or “malignant disease” as described above, more specifically tailored to a corresponding “antigen correlated with said infectious disease” and “antigen correlated with said malignant disease”, respectively.

As regards “antigen correlated with said malignant disease” and “antigen correlated with said infectious disease”, in a preferred embodiment, the antigen is specific for cancer or an antigen specific for infectious diseases.

The person skilled in the art is aware of cell surface structures, epitopes or antigens which are specific for cells that are specific for cancer, or an antigen specific for infectious diseases. Accordingly, the skilled person can choose and select an appropriate antigen specific for cancer or an antigen specific for infectious diseases.

Without being bound to theory, an “antigen correlated with said malignant disease” may, e.g., be a tumor associated antigen (TAA). A variety of tumor-associated antigens are known in the art, including but not limited to carbonic anhydrase IX, CCCL19, CCCL21, CSAp, CD1, CD1a, CD2, CD3, CD4, CD5, CD8, CD11A, CD14, CD15, CD16, CD18, CD19, IGF-1R, CD20, CD21, CD22, CD23, CD25, CD29, CD30, CD32b, CD33, CD37, CD38, CD40, CD40L, CD45, CD46, CD52, CD54, CD55, CD59, CD64, CD66a-e, CD67, CD70, CD74, CD79a, CD80, CD83, CD95, CD126, CD133, CD138, CD147, CD154, AFP, PSMA, CEACAM5, CEACAM6, B7, ED-B of fibronectin, Factor H, FHL-1, Flt-3, folate receptor, GROB, HER2, HMGB-1, hypoxia inducible factor (HIF), HM1.24, insulin-like growth factor-1 (ILGF-1), IFN-γ, IFN-α, IFN-β, IL-2, IL-4R, IL-6R, IL-13R, IL-15R, IL-17R, IL-18R, IL-6, IL-8, IL-12, IL-15, IL-17, IL-18, IL-25, IP-10, mCRP, MCP-1, MIP-1A, MIP-1B, MIF, MUC1, MUC2, MUC3, MUC4, MUC5ac, PAM4 antigen, NCA-95, NCA-90, NY-ESO-1, Ia, HM1.24, EGP-1, EGP-2, HLA-DR, tenascin, Le(y), RANTES, T101, TAC, Tn antigen, Thomson-Friedenreich antigens, tumor necrosis antigens, TNF-α, TRAIL receptor (R1 and R2), VEGFR, EGFR, PlGF, complement factors C3, C3a, C3b, C5a, C5, cancer testis (CT) antigens SPAG9, CT9, CT10, LAGE, MAGE-B5, MAGE-B6, MAGE-C2, MAGE-C3, MAGE-D, HAGE, SAGE and an oncogene product.

Without being bound to theory, an “antigen correlated with said infectious disease” may, e.g., be Epstein-Barr Virus (EBV) (derived) proteins or domains of proteins, preferably, EBNA1, EBNA2, EBNA3A, EBNA3B, EBNA3C, EBNA-LP, LMP1, LMP2A/B, gp350, A73, RPMS1, ZEBRA, Rta, major tegument protein p143, large tegument protein, tegument protein, major capsid protein, minor capsid protein, capsid proteins p18, p23, p40, gN, gp42, gM, gp60, gp78/55, gp150, 53/55kd membrane protein, viral IL-10, gB, gH, gL; Human Papilloma Virus (HPV) (derived) proteins or domains of proteins, preferably E1, E2, E4, E5, E6, E7, L1, L2; Human Immunodeficiency Virus 1 or 2 (derived) proteins or domains of proteins preferably Gag, Pol, Env, gp160, gp120, gp41, CA, MA, p2, p6, NC, IN, RTp66, RTp55, RTp51, PR, Rev, Tat, Nef, Vif, Vpr, Vpu, Vpx.

Herpes Simplex Virus (HSV) 1 or 2 (derived) proteins or domains of proteins, preferably gB, gC, gD, gH, gG, gL, gE, gI, gK, gM, VP1-2, ICP32. ICP0, VP11/12, UL13, vhs, VP16, US3, VP22, ICP34.5, US11, ICP4, DNA polymerase, major capsid protein, helicase, primase, uracil DNA glycosylase, dUTPase, ribonucleotide reductase, large tegument protein; Human Cytomegalovirus (HCMV) (derived) proteins or domains of proteins encoded by open reading frames (ORFs), preferably UL57, UL55, UL54, UL75, UL86, UL85, UL104, UL97, UL98, UL100, UL105, UL102, UL114, UL115, UL72, UL70, UL69, UL44, UL45, UL48; Hepatitis A Virus (HAV) (derived) proteins or domains of proteins, preferably VP1, VP2, VP3, VP4, P2-2A, P2-2B, P2-2C, P3-3A, P3-VPg, P3-3Cpro, P3-3Dpol; Hepatitis B Virus (HBV) (derived) proteins or domains of proteins, preferably M, L, S, polymerase, TP, Spacer, RT, RNaseH, preCore, Core, X, HBeAg; Hepatitis C Virus (HCV) (derived) proteins or domains of proteins, preferably E1, E2, core, NS1, NS2, NS3, NS4a, NS4b, NS5a, NS5b, precursor polyprotein; Influenza A Virus (IAV) (derived) proteins or domains of proteins, preferably HA, NA, M2, M1, NP, NS1, NS2/NEP, PA, PB1; Measles Virus (MV) (derived) proteins or domains of proteins, preferably N, P/C/V, M, F, H, L; Respiratory Syncytial Virus (RSV) (derived) proteins or domains of proteins, preferably NS1, NS2, N, P, M, SH, G, F, M21, M22, L; Rotavirus (derived) proteins or domains of proteins, preferably VP1, VP2, VP3, VP4, RdRp, VP5, VP6, VP7, VP8, NSP1, NSP2, NSP3, NSP4, NSP5, NSP6; Severe acute respiratory syndrome Coronavirus (SARS-Cov) type 1 and 2 (derived) proteins or domains of proteins, preferably ns1, ns2, PLpro, ns4, 3CL, ns6, ns7, ns8, ns9, ns10, RdRp, Hel, ns14, ns15, ns16, S, S-RBD, 3a, E, M, 6, 7a, 7b, 8 N, 9b, 14; Varizella Zoster Virus (VZV) (derived) proteins or domains of proteins or domains of proteins encoded by open reading frames (ORFs), preferably ORF0 to ORF71 (in total at least 71 proteins); Human Herpesvirus type 8 (HHV-8) (derived) proteins or domains of proteins, preferably K1, K2, K3, K4, K4.1, K5, K6, K7, K8, K8.1, K9, K10, K10.5 K11, K12, K13, K14, K15, gB, gL, gH, and proteins encoded in ORFs, preferably ORF2, ORF9, ORF10, ORF16, ORF18, ORF24, ORF30, ORF31, ORF34, ORF66, ORF21, ORF23, ORF25, ORF26, ORF65, ORF33, ORF34, ORF35, ORF36, ORF37, ORF38, ORF39, ORF40, ORF41, ORF42, ORF45, ORF49, ORF50, ORF52, ORF53, ORF55, ORF57, ORF59, ORF67, ORF69, ORF70, ORF72, ORF73, ORF74, ORF75; Human Herpesvirus type 6 (HHV-6) (derived) proteins or domains of proteins encoded by ORFs, preferably DR1, DR6, DR7/U1, U2, U3, U4, U7, U10, U11, U12, U13, U14, U15, U17, U18, U19, U20, U21, U22, U23, U24, U25, U26, U27, U28, U29, U30, U31, U32, U33, U34, U35, U36, U37, U38, U39, U40, U41, U42, U43, U44, U45, U46, U47, U48, U49, U50, U51, U52, U53, U54, U55, U56, U57, U58, U59, U61, U62, U63, U64, U65, U66, U69, U70, U71, U72, U73, U74, U75, U76, U77, U79, U81, U82, U83, U85, U86, U88, U90, U91, U94, U95, U100; Rabies Virus (derived) proteins or domains of proteins, preferably N, P, M, G, L; Mumps Virus (derived) proteins or domains of proteins, preferably N, V/P, M, F, HN, L; and Rhinovirus proteins (derived) or domains of proteins, preferably to VP1, VP2, VP3, VP4, P2-2A, P2-2B, P2-2C, P3-3A, P3-VPg, P3-3C, P3-3D.

Thus, in a preferred embodiment, in the vaccine for use according to the above first aspect, said antigen correlated with said malignant disease is selected from the group consisting of: tumor-associated antigens known in the art, including but not limited to carbonic anhydrase IX, CCCL19, CCCL21, CSAp, CD1, CD1a, CD2, CD3, CD4, CD5, CD8, CD11A, CD14, CD15, CD16, CD18, CD19, IGF-1R, CD20, CD21, CD22, CD23, CD25, CD29, CD30, CD32b, CD33, CD37, CD38, CD40, CD40L, CD45, CD46, CD52, CD54, CD55, CD59, CD64, CD66a-e, CD67, CD70, CD74, CD79a, CD80, CD83, CD95, CD126, CD133, CD138, CD147, CD154, AFP, PSMA, CEACAM5, CEACAM6, B7, ED-B of fibronectin, Factor H, FHL-1, Flt-3, folate receptor, GROB, HER2, HMGB-1, hypoxia inducible factor (HIF), HM1.24, insulin-like growth factor-1 (ILGF-1), IFN-γ, IFN-α, IFN-β, IL-2, IL-4R, IL-6R, IL-13R, IL-15R, IL-17R, IL-18R, IL-6, IL-8, IL-12, IL-15, IL-17, IL-18, IL-25, IP-10, mCRP, MCP-1, MIP-1A, MIP-1B, MIF, MUC1, MUC2, MUC3, MUC4, MUC5ac, PAM4 antigen, NCA-95, NCA-90, NY-ESO-1, Ia, HM1.24, EGP-1, EGP-2, HLA-DR, tenascin, Le(y), RANTES, T101, TAC, Tn antigen, Thomson-Friedenreich antigens, tumor necrosis antigens, TNF-α, TRAIL receptor (R1 and R2), VEGFR, EGFR, PlGF, complement factors C3, C3a, C3b, C5a, C5, cancer testis (CT) antigens SPAG9, CT9, CT10, LAGE, MAGE-B5, MAGE-B6, MAGE-C2, MAGE-C3, MAGE-D, HAGE, SAGE and an oncogene product.

Moreover, regarding the term “antigen correlated with said infectious disease”, in a preferred embodiment, in the vaccine for use according to the above first aspect, said antigen correlated with said infectious disease is selected from the group consisting of:
Epstein-Barr Virus (EBV) (derived) proteins or domains of proteins, preferably EBNA1, EBNA2, EBNA3A, EBNA3B, EBNA3C, EBNA-LP, LMP1, LMP2A/B, gp350, A73, RPMS1, ZEBRA, Rta, major tegument protein p143, large tegument protein, tegument protein, major capsid protein, minor capsid protein, capsid proteins p18, p23, p40, gN, gp42, gM, gp60, gp78/55, gp150, 53/55kd membrane protein, viral IL-10, gB, gH, gL;
Human Papilloma Virus (HPV) (derived) proteins or domains of proteins, preferably E1, E2, E4, E5, E6, E7, L1, L2;
Human Immunodeficiency Virus 1 or 2 (derived) proteins or domains of proteins, preferably to Gag, Pol, Env, gp160, gp120, gp41, CA, MA, p2, p6, NC, IN, RTp66, RTp55, RTp51, PR, Rev, Tat, Nef, Vif, Vpr, Vpu, Vpx;
Herpes Simplex Virus (HSV) 1 or 2 (derived) proteins or domains of proteins, preferably to gB, gC, gD, gH, gG, gL, gE, gI, gK, gM, VP1-2, ICP32. ICP0, VP11/12, UL13, vhs, VP16, US3, VP22, ICP34.5, US11, ICP4, DNA polymerase, major capsid protein, helicase, primase, uracil DNA glycosylase, dUTPase, ribonucleotide reductase, large tegument protein;
Human Cytomegalovirus (HCMV) (derived) proteins or domains of proteins encoded by open reading frames (ORFs), preferably UL57, UL55, UL54, UL75, UL86, UL85, UL104, UL97, UL98, UL100, UL105, UL102, UL114, UL115, UL72, UL70, UL69, UL44, UL45, UL48;
Hepatitis A Virus (HAV) (derived) proteins or domains of proteins, preferably VP1, VP2, VP3, VP4, P2-2A, P2-2B, P2-2C, P3-3A, P3-VPg, P3-3Cpro, P3-3Dpol;
Hepatitis B Virus (HBV) (derived) proteins or domains of proteins, preferably to M, L, S, polymerase, TP, Spacer, RT, RNaseH, preCore, Core, X, HBeAg;
Hepatitis C Virus (HCV) (derived) proteins or domains of proteins, preferably E1, E2, core, NS1, NS2, NS3, NS4a, NS4b, NS5a, NS5b, precursor polyprotein;
Influenza A Virus (IAV) (derived) proteins or domains of proteins, preferably HA, NA, M2, M1, NP, NS1, NS2/NEP, PA, PB1;
Measles Virus (MV) (derived) proteins or domains of proteins, preferably N, P/C/V, M, F, H, L;
Respiratory Syncytial Virus (RSV) (derived) proteins or domains of proteins, preferably NS1, NS2, N, P, M, SH, G, F, M21, M22, L;
Rotavirus (derived) proteins or domains of proteins, preferably VP1, VP2, VP3, VP4, RdRp, VP5, VP6, VP7, VP8, NSP1, NSP2, NSP3, NSP4, NSP5, NSP6;
Severe acute respiratory syndrome Coronavirus (SARS-Cov) type 1 and 2 (derived) proteins or domains of proteins, preferably ns1, ns2, PLpro, ns4, 3CL, ns6, ns7, ns8, ns9, ns10, RdRp, Hel, ns14, ns15, ns16, S, S-RBD, 3a, E, M, 6, 7a, 7b, 8 N, 9b, 14;
Varizella Zoster Virus (VZV) (derived) proteins or domains of proteins or domains of proteins encoded by open reading frames (ORFs), preferably ORF0 to ORF71 (in total at least 71 proteins);
Human Herpesvirus type 8 (HHV-8) (derived) proteins or domains of proteins, preferably K1, K2, K3, K4, K4.1, K5, K6, K7, K8, K8.1, K9, K10, K10.5 K11, K12, K13, K14, K15, gB, gL, gH, and proteins encoded in ORFs including but not limited to ORF2, ORF9, ORF10, ORF16, ORF18, ORF24, ORF30, ORF31, ORF34, ORF66, ORF21, ORF23, ORF25, ORF26, ORF65, ORF33, ORF34, ORF35, ORF36, ORF37, ORF38, ORF39, ORF40, ORF41, ORF42, ORF45, ORF49, ORF50, ORF52, ORF53, ORF55, ORF57, ORF59, ORF67, ORF69, ORF70, ORF72, ORF73, ORF74, ORF75;
Human Herpesvirus type 6 (HHV-6) (derived) proteins or domains of proteins encoded by ORFs, preferably DR1, DR6, DR7/U1, U2, U3, U4, U7, U10, U11, U12, U13, U14, U15, U17, U18, U19, U20, U21, U22, U23, U24, U25, U26, U27, U28, U29, U30, U31, U32, U33, U34, U35, U36, U37, U38, U39, U40, U41, U42, U43, U44, U45, U46, U47, U48, U49, U50, U51, U52, U53, U54, U55, U56, U57, U58, U59, U61, U62, U63, U64, U65, U66, U69, U70, U71, U72, U73, U74, U75, U76, U77, U79, U81, U82, U83, U85, U86, U88, U90, U91, U94, U95, U100;
Rabies Virus (derived) proteins or domains of proteins, preferably N, P, M, G, L;
Mumps Virus (derived) proteins or domains of proteins, preferably N, V/P, M, F, HN, L; and
Rhinovirus (derived) proteins or domains of proteins, preferably VP1, VP2, VP3, VP4, P2-2A, P2-2B, P2-2C, P3-3A, P3-VPg, P3-3C, P3-3D.

As mentioned above, the vaccine for use in actively immunizing a subject against an infectious disease or malignant disease requires, in a preferred embodiment, the presence of an acute infection, i.e., the presence of antigen. This, in particular, relates to the aspect (a) wherein an antibody against an antigen correlated with said infectious disease or malignant disease is used as a component of the vaccine of the present invention. This is because, for being able actively immunizing the subject in line with the rationale of the present invention, not only the antibody but also an antigen correlated with said infectious disease or malignant disease must be present. Regarding aspect (b), an active infection is not necessarily required as the antigen is fused to Fc and, accordingly, the respective antigen is readily present even in the absence of an acute infection.

“Acute” in this respect means that the subject has a breakout of the infectious disease or malignant disease and, accordingly, antigens correlated with said infectious disease or malignant disease are present in the body of the subject.

In a more preferred embodiment, “acute” means that the subject shows symptoms of the infectious disease or malignant disease. In other words, the subject to be vaccinated is in actual need of a vaccination or treatment and the term “acute” relates to situations after the (primary) onset of the infectious disease or malignant disease. The term “acute” as referred to in the context of the present invention is opposed to a prophylactic or preventive, i.e., measures taken for disease prevention, e.g., in order to prevent the infection and/or the onset/outbreak of the disease. More specifically, prophylactic treatment may be understood in a way that it prevents attachment of free virus particles (from outside the body) to target cells and in turn prevents virus replication.

In the context of the aspect wherein an Fc-containing fusion protein comprising an Fc part of an antibody fused to an antigen correlated with said infectious disease or malignant disease is used as a component of the vaccine of the present invention, does not necessarily require for its active immunization of a subject against an infectious disease or malignant disease in terms of the present invention the presence of an acute infection. This, in particular, relates to the aspect wherein an antibody against an antigen correlated with said infectious disease or malignant disease is used as a component of the vaccine of the present invention. This is because, for being able actively immunizing the subject in line with the rationale of the present invention, not only the antibody but also an antigen correlated with said infectious disease or malignant disease must be present. However, in the context of the aspect wherein an Fc-containing fusion protein comprising an Fc part of an antibody fused to an antigen correlated with said infectious disease or malignant disease is used as a component of the vaccine of the present invention, the antigen correlated with said infectious disease or malignant disease is already there, i.e., part of the Fc-containing fusion protein and, accordingly, is already comprised in the vaccine.

As already outlined above, the vaccine of the present invention may comprise one of the two alternative components (while, it is also contemplated that the vaccine comprises both components), i.e.,
(a) an antibody against an antigen correlated with said infectious disease or malignant disease; or
(b) an Fc-containing fusion protein comprising an Fc part of an antibody fused to an antigen correlated with said infectious disease or malignant disease, wherein said Fc part is capable of binding a receptor present on or in an antigen presenting cell (APC) selected from the group consisting of:
   Type I: activatory FcγRI, FcγRIIa, FcγRIIc, FcγRIIIa, FcγRIIIb, and inhibitory FcγRIIb; and
   Type II: neonatal FcR (FcRn) and cytosolic TRIM21.

These two components are separately described in more detail in the following.

An Antibody Against an Antigen Correlated with Said Infectious Disease or Malignant Disease:

As regards (a), i.e., an antibody against an antigen correlated with said infectious disease or malignant disease, the term “antibody” is known in the art and is often also referred to as an immunoglobulin (Ig).

In the context of the present invention, in line with the rationale of the present invention, the term “antibody” refers to an antibody which comprises at least one Fc-part of an antibody. Accordingly, in a preferred embodiment, the antibody of component (a) of the vaccine of the invention described above (i.e., the antibody against an antigen correlated with said infectious disease or malignant disease) comprises at least one Fc-part of an antibody.

An “Fc”-part or “Fc”-region of an antibody contains two heavy chain fragments comprising the C_{H}2 and C_{H}3 domains of an antibody. The two heavy chain fragments are held together by two or more disulfide bonds and by hydrophobic interactions of the C_{H}3 domains.

In the context of the present invention, the term “antibody” relates to full immunoglobulin molecules as well as to parts of such immunoglobulin molecules (i.e., “antigen-binding fragment thereof”).

Furthermore, the term relates, as outlined in more detail below, to modified and/or altered antibody molecules. The term also relates to recombinantly or synthetically generated/synthesized antibodies. The term also relates to intact antibodies as well as to antibody fragments thereof, like, separated light and heavy chains.

As the antibody of the present invention comprises at least one Fc-part of an antibody, Fab, Fv, Fab′, Fab′-SH, F(ab′)2 are not envisaged.

The term antibody also comprises but is not limited to fully-human antibodies, chimeric antibodies, humanized antibodies, CDR-grafted antibodies and antibody constructs, like single chain Fvs (scFv) or antibody-fusion proteins.

In a preferred embodiment, the antibody according the present invention is a monoclonal antibody. In a further preferred embodiment, the according to the present invention is a humanized or a fully human antibody. In a further preferred embodiment, the antibody according to the present invention is a murine antibody.

The term “monoclonal antibody” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Monoclonal antibodies are advantageous in that they may be synthesized by a hybridoma culture, essentially uncontaminated by other immunoglobulins. The modified “monoclonal” indicates the character of the antibody as being amongst a substantially homogeneous population of antibodies, and is not to be construed as requiring production of the antibody by any particular method. As mentioned above, the monoclonal antibodies to be used in accordance with the present invention may be made by the hybridoma method described by Kohler, Nature 256 (1975), 495.

The term “polyclonal antibody” as used herein, refers to an antibody which was produced among or in the presence of one or more other, non-identical antibodies. In general, polyclonal antibodies are produced from a B-lymphocyte in the presence of several other B-lymphocytes which produced non-identical antibodies. Usually, polyclonal antibodies are obtained directly from an immunized animal.

The term “fully-human antibody” as used herein refers to an antibody which comprises human immunoglobulin protein sequences only. A fully human antibody may contain murine carbohydrate chains if produced in a mouse, in a mouse cell or in a hybridoma derived from a mouse cell. Similarly, “mouse antibody” or “murine antibody” refers to an antibody which comprises mouse/murine immunoglobulin protein sequences only. Alternatively, a “fully-human antibody” may contain rat carbohydrate chains if produced in a rat, in a rat cell, in a hybridoma derived from a rat cell. Similarly, the term “rat antibody” refers to an antibody that comprises rat immunoglobulin sequences only. Fully-human antibodies may also be produced, for example, by phage display which is a widely used screening technology which enables production and screening of fully human antibodies. Also phage antibodies can be used in context of this invention. Phage display methods are described, for example, in U.S. Pat. Nos. 5,403,484, 5,969,108 and 5,885,793. Another technology which enables development of fully-human antibodies involves a modification of mouse hybridoma technology. Mice are made transgenic to contain the human immunoglobulin locus in exchange for their own mouse genes (see, for example, U.S. Pat. No. 5,877,397).

The term “chimeric antibodies”, refers to an antibody which comprises a variable region of the present invention fused or chimerized with an antibody region (e.g., constant region) from another, human or non-human species (e.g., mouse, horse, rabbit, dog, cow, chicken).

The term antibody also relates to recombinant human antibodies, heterologous antibodies and heterohybrid antibodies. The term “recombinant human antibody” includes all human sequence antibodies that are prepared, expressed, created or isolated by recombinant means, such as antibodies isolated from an animal (e.g., a mouse) that is transgenic for human immunoglobulin genes; antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences.

Such recombinant human antibodies have variable and constant regions (if present) derived from human germline immunoglobulin sequences. Such antibodies can, however, be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

A “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

The term “heterohybrid antibody” refers to an antibody having light and heavy chains of different organismal origins. For example, an antibody having a human heavy chain associated with a murine light chain is a heterohybrid antibody. Examples of heterohybrid antibodies include chimeric and humanized antibodies.

The term antibody also relates to humanized antibodies. “Humanized” forms of non-human (e.g. murine or rabbit) antibodies are chimeric immunoglobulins, immunoglobulin chains or fragments thereof which contain minimal sequence derived from non-human immunoglobulin. Often, humanized antibodies are human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Furthermore, humanized antibody may comprise residues, which are found neither in the recipient antibody nor in the imported CDR or framework sequences. These modifications are made to further refine and optimize antibody performance. In general, the humanized antibody will comprise substantially all of at least one, and typically two variable domains, in which all or substantially all of the CDR regions correspond to those of a non-human immunoglobulin and all or substantially all of the FR regions are those of a human immunoglobulin consensus sequence. The humanized antibody may also comprise at least a portion of an immunoglobulin constant region (Fc), typically that of a human immunoglobulin. For further details, see: Jones Nature 321 (1986), 522-525; Reichmann Nature 332 (1998), 323-327 and Presta Curr Op Struct Biol 2 (1992), 593-596.

A popular method for humanization of antibodies involves CDR grafting, where a functional antigen-binding site from a non-human ‘donor’ antibody is grafted onto a human ‘acceptor’ antibody. Moreover, humanization of antibodies involves the substitution of amino acid residues in framework regions or in the grated CDRs in order to improve the fitting/matching of the grafted CDRs with the framework scaffold of the acceptor variable regions. Such a fitting/matching typically restores the initial affinity of the donor antibody or even improves the same; see, e.g., EP 0 451 216. CDR grafting methods are known in the art and described, for example, in U.S. Pat. Nos. 5,225,539, 5,693,761 and 6,407,213. Another related method is the production of humanized antibodies from transgenic animals that are genetically engineered to contain one or more humanized immunoglobulin loci which are capable of undergoing gene rearrangement and gene conversion (see, for example, U.S. Pat. No. 7,129,084).

Accordingly, in context of the present invention, the term “antibody” relates to full immunoglobulin molecules as well as to parts of such immunoglobulin molecules.

Yet, as mentioned above, the antibody preferably contains an Fc-region of an antibody.

Furthermore, the term relates, as discussed above, to modified and/or altered antibody molecules. The term also relates to recombinantly or synthetically generated/synthesized antibodies. The term also relates to intact antibodies.

As the antibody of the present invention comprises at least one Fc-part of an antibody, the term does not relate to intact antibodies or to antibody fragments thereof, like, separated light and heavy chains, Fab, Fv, Fab′, Fab′-SH, F(ab′)2.

The term antibody also comprises but is not limited to fully-human antibodies, chimeric antibodies, humanized antibodies, CDR-grafted antibodies and antibody constructs, like single chain Fvs (scFv) or antibody-fusion proteins.

In a preferred embodiment, the antibody in terms of the present invention is a full-length antibody, i.e., a full immunoglobulin molecule which is often also referred to as complete antibody.

“Single-chain Fv” or “scFv” antibody fragments have, in the context of the invention, the V_{H} and V_{L} domains of an antibody, wherein these domains are present in a single polypeptide chain. Generally, the scFv polypeptide further comprises a polypeptide linker between the V_{H} and V_{L} domains which enables the scFv to form the desired structure for antigen binding. Techniques described for the production of single chain antibodies are described, e.g., in Plückthun in The Pharmacology of Monoclonal Antibodies, Rosenburg and Moore eds. Springer-Verlag, N.Y. (1994), 269-315.

A “Fab fragment” as used herein is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}2 and C_{H}3 domains of an antibody. The two heavy chain fragments are held together by two or more disulfide bonds and by hydrophobic interactions of the C_{H}3 domains.

A “Fab′ fragment” as described in the context of the invention contains one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

A “F(ab′)₂ fragment” as described in the context of the invention contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” as described in the context of the invention comprises the variable regions from both the heavy and light chains, but lacks the constant regions.

Antibodies, antibody constructs, antibody fragments, antibody derivatives (all being Ig-derived) to be employed in accordance with the invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook (1989), loc. cit. The term “Ig-derived domain” particularly relates to (poly) peptide constructs comprising at least one CDR. Fragments or derivatives of the recited Ig-derived domains define (poly) peptides which are parts of the above antibody molecules and/or which are modified by chemical/biochemical or molecular biological methods. Corresponding methods are known in the art and described inter alia in laboratory manuals (see Sambrook et al., Molecular Cloning: A Laboratory Manual; Cold Spring Harbor Laboratory Press, 2nd edition (1989) and 3rd edition (2001); Gerhardt et al., Methods for General and Molecular Bacteriology ASM Press (1994); Lefkovits, Immunology Methods Manual: The Comprehensive Sourcebook of Techniques; Academic Press (1997); Golemis, Protein-Protein Interactions: A Molecular Cloning Manual Cold Spring Harbor Laboratory Press (2002)).

As already outlined above, in line with the rationale of the present invention, the present invention is not particularly limited to a certain “infectious disease” or a certain “malignant disease”. As a direct consequence thereof, it is understood by the skilled person that the vaccine of the present invention is not particularly limited to an “antibody against a particular antigen correlated with a infectious disease or malignant disease”.

Therefore, the term “antigen correlated with said infectious disease or malignant disease” in terms of the present invention is not particularly limited to a specific antigen but can be any antigen which correlates with said infectious disease or malignant disease.

The “antigen correlated with said infectious disease” may be derived from the above-described infectious disease-causing agent (like, e.g., bacteria, viruses, fungi or parasites as described above), i.e., may be, e.g., a part or structure or domain of the infectious disease-causing agent which is capable of eliciting an immune response.

The “antigen correlated with said malignant disease” may be derived from the above-described malignant diseases, i.e., may be, e.g., a part or structure or domain of malignant cell which is capable of eliciting an immune response.

Accordingly, it is understood by the skilled person that the vaccine of the present invention can be tailored to any “infectious disease” or “malignant disease”, more specifically tailored to a corresponding “antigen correlated with said infectious disease” and “antigen correlated with said malignant disease”, respectively.

Thus, in a preferred embodiment, the “antibody against an antigen correlated with a infectious disease or a malignant disease” in terms of the present invention is an antibody which specifically binds to or specifically recognizes or interacts with “antigen correlated with said infectious disease” and “antigen correlated with said malignant disease”, respectively. The terms “binding to”, “specifically binding to”, “specifically recognizing” or “specifically interacting” as used in the context of the present invention are used herein interchangeably and are further defined further below.

Accordingly, the antibody as used in the context of the present invention, is not particularly limited as long as it is an antibody against an “antigen correlated with said infectious disease” and “antigen correlated with said malignant disease”, respectively.

Thus, the antibody may be any antibody which specifically binds to or specifically recognizes or interacts with an “antigen that correlates with said infectious disease” and “antigen that correlates with said malignant disease”, respectively. Preferably, the antigen, i.e., the domain, is a surface-antigen.

The skilled person is readily in a position to generate such an antibody directed to a given domain (i.e., an “antigen correlated with said infectious disease” and “antigen correlated with said malignant disease”, respectively) and determine whether a respective antibody is capable of detecting/binding to a given “antigen correlated with said infectious disease” and “antigen correlated with said malignant disease”, respectively, based on the skilled person's common general knowledge and the methods described above.

The generation of specific antibodies against an “antigen correlated with said infectious disease” and “antigen correlated with said malignant disease”, respectively, may be based, for example, on the immunization of animals, like mice. However, also other animals for the production of antibody/antisera are envisaged within the present invention. For example, monoclonal and polyclonal antibodies can be produced by rabbit, mice, goats, donkeys and the like. The polynucleotide encoding a correspondingly chosen polypeptide of the “antigen correlated with said infectious disease” and the “antigen correlated with said malignant disease”, respectively can be subcloned into an appropriate vector, wherein the recombinant polypeptide is to be expressed in an organism being able for an expression, for example in bacteria. Thus, the expressed recombinant protein can be intra-peritoneally injected into a mice and the resulting specific antibody can be, for example, obtained from the mice serum being provided by intra-cardiac blood puncture. The present invention also envisages the production of specific antibodies against native polypeptides and recombinant polypeptides by using a DNA vaccine strategy as exemplified in the appended examples. DNA vaccine strategies are well-known in the art and encompass liposome-mediated delivery, by gene gun or jet injection and intramuscular or intradermal injection. Thus, antibodies directed against a polypeptide or a protein or an epitope of an “antigen correlated with said infectious disease” and an “antigen correlated with said malignant disease”, respectively, can be obtained by directly immunizing the animal by directly injecting intramuscularly the vector expressing the desired polypeptide or a protein or an epitope of an “antigen correlated with said infectious disease” and an “antigen correlated with said malignant disease”, respectively. The amount of obtained specific antibody can be quantified using an ELISA, which is also described herein below. Further methods for the production of antibodies are well known in the art, see, e.g. Harlow and Lane, “Antibodies, A Laboratory Manual”, CSH Press, Cold Spring Harbor, 1988.

The term “specifically binds”, as used herein, refers to a binding reaction that is determinative of the presence of the desired “antigen correlated with said infectious disease” and “antigen correlated with said malignant disease”, respectively, and an antibody in the presence of a heterogeneous population of proteins and other biologics.

Thus, under designated assay conditions, the specified antibodies and a corresponding “antigen correlated with said infectious disease” and “antigen correlated with said malignant disease”, respectively, bind to one another and do not bind in a significant amount to other components present in a sample. Specific binding to a target analyte under such conditions may require a binding moiety that is selected for its specificity for a particular target analyte. A variety of immunoassay formats may be used to select antibodies specifically reactive with a particular antigen. For example, solid-phase ELISA immunoassays are routinely used to select monoclonal antibodies specifically immunoreactive with an analyte. See Shepherd and Dean (2000), Monoclonal Antibodies: A Practical Approach, Oxford University Press and/or Howard and Bethell (2000) Basic Methods in Antibody Production and Characterization, Crc. Pr. Inc. for a description of immunoassay formats and conditions that can be used to determine specific immunoreactivity. Typically a specific or selective reaction will be at least twice background signal to noise and more typically more than 10 to 100 times greater than background. The person skilled in the art is in a position to provide for and generate specific binding molecules directed against the novel polypeptides. For specific binding-assays it can be readily employed to avoid undesired cross-reactivity, for example polyclonal antibodies can easily be purified and selected by known methods (see Shepherd and Dean, loc. cit.).

The term “antibody against an antigen correlated with said infectious disease or malignant disease” means in accordance with this invention that the antibody molecule or antigen-binding fragment thereof is capable of specifically recognizing or specifically interacting with and/or binding to at least two amino acids of the “antigen correlated with said infectious disease” and “antigen correlated with said malignant disease”, respectively. Said term relates to the specificity of the antibody molecule, i.e. to its ability to discriminate between the specific regions of the “antigen correlated with said infectious disease” and “antigen correlated with said malignant disease”, respectively. Accordingly, specificity can be determined experimentally by methods known in the art and methods as disclosed and described herein. Such methods comprise, but are not limited to Western blots, ELISA-, RIA-, ECL-, IRMA-tests and peptide scans. Such methods also comprise the determination of K_{D}-values as, inter alia, illustrated in the appended examples. The peptide scan (pepspot assay) is used routinely employed to map linear epitopes in a polypeptide antigen. The primary sequence of the polypeptide is synthesized successively on activated cellulose with peptides overlapping one another. The recognition of certain peptides by the antibody to be tested for its ability to detect or recognize a specific antigen/epitope is scored by routine colour development (secondary antibody with horseradish peroxide and 4-chloronaphthol and hydrogen peroxide), by a chemoluminescence reaction or similar means known in the art. In the case of, inter alia, chemoluminescence reactions, the reaction can be quantified. If the antibody reacts with a certain set of overlapping peptides one can deduce the minimum sequence of amino acids that are necessary for reaction. The same assay can reveal two distant clusters of reactive peptides, which indicate the recognition of a discontinuous, i.e. conformational epitope in the antigenic polypeptide (Geysen (1986), Mol. Immunol. 23, 709-715).

The term “binding to” as used in the context of the present invention defines a binding (interaction) of at least two “antigen-interaction-sites” with each other. The term “antigen-interaction-site” defines, in accordance with the present invention, a motif of a polypeptide, i.e., a part of the antibody or antigen-binding fragment of the present invention, which shows the capacity of specific interaction with a specific antigen or a specific group of antigens of the “antigen correlated with said infectious disease” and “antigen correlated with said malignant disease”, respectively. Said binding/interaction is also understood to define a “specific recognition”. The term “specifically recognizing” means in accordance with this invention that the antibody is capable of specifically interacting with and/or binding to at least two amino acids of each of an “antigen correlated with said infectious disease” and an “antigen correlated with said malignant disease”, respectively as defined herein. Antibodies can recognize, interact and/or bind to different epitopes.

The term “specific interaction” as used in accordance with the present invention means that the antibody or antigen-binding fragment thereof of the invention does not or does not essentially cross-react with (poly) peptides of similar structures. Accordingly, the antibody or antigen-binding fragment thereof of the invention specifically binds to/interacts with structures of an “antigen correlated with said infectious disease” and an “antigen correlated with said malignant disease”, respectively, as defined above. Specific examples of such molecules against which said first and second, Ig-derived domain is directed are given herein below.

Cross-reactivity of a panel of antibodies or antigen-binding fragments thereof under investigation may be tested, for example, by assessing binding of said panel of antibody or antigen-binding fragment thereof under conventional conditions (see, e.g., Harlow and Lane, Antibodies: A Laboratory Manual, Cold Spring Harbor Laboratory Press, (1988) and Using Antibodies: A Laboratory Manual, Cold Spring Harbor Laboratory Press, (1999)) to the (poly)peptide of interest as well as to a number of more or less (structurally and/or functionally) closely related (poly)peptides. Only those constructs (i.e. antibodies, antigen-binding fragments thereof and the like) that bind to the certain structure of the HSV, e.g., a specific epitope or (poly) peptide/protein of the HSV but do not or do not essentially bind to any of the other epitope or (poly) peptides of the same HSV, are considered specific for the epitope or (poly) peptide/protein of interest and selected for further studies in accordance with the method provided herein. These methods may comprise, inter alia, binding studies, blocking and competition studies with structurally and/or functionally closely related molecules. These binding studies also comprise FACS analysis, surface plasmon resonance (SPR, e.g. with BIAcore®), analytical ultracentrifugation, isothermal titration calorimetry, fluorescence anisotropy, fluorescence spectroscopy or by radiolabeled ligand binding assays.

The term “binding to” does not only relate to a linear epitope but may also relate to a conformational epitope, a structural epitope or a discontinuous epitope consisting of two regions of the human target molecules or parts thereof. In the context of this invention, a conformational epitope is defined by two or more discrete amino acid sequences separated in the primary sequence which comes together on the surface of the molecule when the polypeptide folds to the native protein (Sela, Science 166 (1969), 1365 and Laver, Cell 61 (1990), 553-536). Moreover, the term “binding to” is interchangeably used in the context of the present invention with the terms “interacting with” or “recognizing”.

Accordingly, specificity can be determined experimentally by methods known in the art and methods as described herein. Such methods comprise, but are not limited to Western Blots, ELISA-, RIA-, ECL-, IRMA-tests and peptide scans.

An Fc-Containing Fusion Protein Comprising an Fc Part of an Antibody Fused to an Antigen Correlated with Said Infectious Disease or Malignant Disease:

As regards (b), i.e., an Fc-containing fusion protein comprising an Fc part of an antibody fused to an antigen correlated with said infectious disease or malignant disease, wherein said Fc part is capable of binding a receptor present on or in an antigen presenting cell (APC) selected from the group consisting of: Type I: activatory FcγRI, FcγRIIa, FcγRIIc, FcγRIIIa, FcγRIIIb, and inhibitory FcγRIIb; and Type II: neonatal FcR (FcRn) and cytosolic TRIM21.

The fusion protein according the (b) comprises at least two modules, i.e., an “Fc-part of an antibody” as well as “antigen correlated with said infectious disease or malignant disease”.

The term “antigen correlated with said infectious disease or malignant disease” has already been defined above in the context of (a), i.e., in the context of the antibody against and “antigen correlated with said infectious disease or malignant disease”.

As regards the preferred embodiments of the “antigen correlated with said infectious disease or malignant disease” the same applies, mutatis mutandis, as has been set forth above in the context of the antibody against and “antigen correlated with said infectious disease or malignant disease” as defined above.

Thus, in a preferred embodiment, in the vaccine for use according to the above first aspect, said antigen correlated with said malignant disease is selected from the group consisting of: tumor-associated antigens known in the art, including but not limited to carbonic anhydrase IX, CCCL19, CCCL21, CSAp, CD1, CD1a, CD2, CD3, CD4, CD5, CD8, CD11A, CD14, CD15, CD16, CD18, CD19, IGF-1R, CD20, CD21, CD22, CD23, CD25, CD29, CD30, CD32b, CD33, CD37, CD38, CD40, CD40L, CD45, CD46, CD52, CD54, CD55, CD59, CD64, CD66a-e, CD67, CD70, CD74, CD79a, CD80, CD83, CD95, CD126, CD133, CD138, CD147, CD154, AFP, PSMA, CEACAM5, CEACAM6, B7, ED-B of fibronectin, Factor H, FHL-1, Flt-3, folate receptor, GROB, HER2, HMGB-1, hypoxia inducible factor (HIF), HM1.24, insulin-like growth factor-1 (ILGF-1), IFN-γ, IFN-α, IFN-β, IL-2, IL-4R, IL-6R, IL-13R, IL-15R, IL-17R, IL-18R, IL-6, IL-8, IL-12, IL-15, IL-17, IL-18, IL-25, IP-10, mCRP, MCP-1, MIP-1A, MIP-1B, MIF, MUC1, MUC2, MUC3, MUC4, MUC5ac, PAM4 antigen, NCA-95, NCA-90, NY-ESO-1, Ia, HM1.24, EGP-1, EGP-2, HLA-DR, tenascin, Le(y), RANTES, T101, TAC, Tn antigen, Thomson-Friedenreich antigens, tumor necrosis antigens, TNF-α, TRAIL receptor (R1 and R2), VEGFR, EGFR, PlGF, complement factors C3, C3a, C3b, C5a, C5, cancer testis (CT) antigens SPAG9, CT9, CT10, LAGE, MAGE-B5, MAGE-B6, MAGE-C2, MAGE-C3, MAGE-D, HAGE, SAGE and an oncogene product.

Moreover, regarding the term “antigen correlated with said infectious disease”, in a preferred embodiment, in the vaccine for use according to the above first aspect, said antigen correlated with said infectious disease is selected from the group consisting of:
Epstein-Barr Virus (EBV) (derived) proteins or domains of proteins, preferably EBNA1, EBNA2, EBNA3A, EBNA3B, EBNA3C, EBNA-LP, LMP1, LMP2A/B, gp350, A73, RPMS1, ZEBRA, Rta, major tegument protein p143, large tegument protein, tegument protein, major capsid protein, minor capsid protein, capsid proteins p18, p23, p40, gN, gp42, gM, gp60, gp78/55, gp150, 53/55kd membrane protein, viral IL-10, gB, gH, gL;
Human Papilloma Virus (HPV) (derived) proteins or domains of proteins, preferably E1, E2, E4, E5, E6, E7, L1, L2;
Human Immunodeficiency Virus 1 or 2 (derived) proteins or domains of proteins, preferably to Gag, Pol, Env, gp160, gp120, gp41, CA, MA, p2, p6, NC, IN, RTp66, RTp55, RTp51, PR, Rev, Tat, Nef, Vif, Vpr, Vpu, Vpx;
Herpes Simplex Virus (HSV) 1 or 2 (derived) proteins or domains of proteins, preferably to gB, gC, gD, gH, gG, gL, gE, gI, gK, gM, VP1-2, ICP32. ICP0, VP11/12, UL13, vhs, VP16, US3, VP22, ICP34.5, US11, ICP4, DNA polymerase, major capsid protein, helicase, primase, uracil DNA glycosylase, dUTPase, ribonucleotide reductase, large tegument protein;
Human Cytomegalovirus (HCMV) (derived) proteins or domains of proteins encoded by open reading frames (ORFs), preferably UL57, UL55, UL54, UL75, UL86, UL85, UL104, UL97, UL98, UL100, UL105, UL102, UL114, UL115, UL72, UL70, UL69, UL44, UL45, UL48;
Hepatitis A Virus (HAV) (derived) proteins or domains of proteins, preferably VP1, VP2, VP3, VP4, P2-2A, P2-2B, P2-2C, P3-3A, P3-VPg, P3-3Cpro, P3-3Dpol;
Hepatitis B Virus (HBV) (derived) proteins or domains of proteins, preferably to M, L, S, polymerase, TP, Spacer, RT, RNaseH, preCore, Core, X, HBeAg;
Hepatitis C Virus (HCV) (derived) proteins or domains of proteins, preferably E1, E2, core, NS1, NS2, NS3, NS4a, NS4b, NS5a, NS5b, precursor polyprotein;
Influenza A Virus (IAV) (derived) proteins or domains of proteins, preferably HA, NA, M2, M1, NP, NS1, NS2/NEP, PA, PB1;
Measles Virus (MV) (derived) proteins or domains of proteins, preferably N, P/C/V, M, F, H, L;
Respiratory Syncytial Virus (RSV) (derived) proteins or domains of proteins, preferably NS1, NS2, N, P, M, SH, G, F, M21, M22, L;
Rotavirus (derived) proteins or domains of proteins, preferably VP1, VP2, VP3, VP4, RdRp, VP5, VP6, VP7, VP8, NSP1, NSP2, NSP3, NSP4, NSP5, NSP6;
Severe acute respiratory syndrome Coronavirus (SARS-Cov) type 1 and 2 (derived) proteins or domains of proteins, preferably ns1, ns2, PLpro, ns4, 3CL, ns6, ns7, ns8, ns9, ns10, RdRp, Hel, ns14, ns15, ns16, S, S-RBD, 3a, E, M, 6, 7a, 7b, 8 N, 9b, 14;
Varizella Zoster Virus (VZV) (derived) proteins or domains of proteins or domains of proteins encoded by open reading frames (ORFs), preferably ORF0 to ORF71 (in total at least 71 proteins);
Human Herpesvirus type 8 (HHV-8) (derived) proteins or domains of proteins, preferably K1, K2, K3, K4, K4.1, K5, K6, K7, K8, K8.1, K9, K10, K10.5 K11, K12, K13, K14, K15, gB, gL, gH, and proteins encoded in ORFs including but not limited to ORF2, ORF9, ORF10, ORF16, ORF18, ORF24, ORF30, ORF31, ORF34, ORF66, ORF21, ORF23, ORF25, ORF26, ORF65, ORF33, ORF34, ORF35, ORF36, ORF37, ORF38, ORF39, ORF40, ORF41, ORF42, ORF45, ORF49, ORF50, ORF52, ORF53, ORF55, ORF57, ORF59, ORF67, ORF69, ORF70, ORF72, ORF73, ORF74, ORF75;
Human Herpesvirus type 6 (HHV-6) (derived) proteins or domains of proteins encoded by ORFs, preferably DR1, DR6, DR7/U1, U2, U3, U4, U7, U10, U11, U12, U13, U14, U15, U17, U18, U19, U20, U21, U22, U23, U24, U25, U26, U27, U28, U29, U30, U31, U32, U33, U34, U35, U36, U37, U38, U39, U40, U41, U42, U43, U44, U45, U46, U47, U48, U49, U50, U51, U52, U53, U54, U55, U56, U57, U58, U59, U61, U62, U63, U64, U65, U66, U69, U70, U71, U72, U73, U74, U75, U76, U77, U79, U81, U82, U83, U85, U86, U88, U90, U91, U94, U95, U100;
Rabies Virus (derived) proteins or domains of proteins, preferably N, P, M, G, L;
Mumps Virus (derived) proteins or domains of proteins, preferably N, V/P, M, F, HN, L; and
Rhinovirus (derived) proteins or domains of proteins, preferably VP1, VP2, VP3, VP4, P2-2A, P2-2B, P2-2C, P3-3A, P3-VPg, P3-3C, P3-3D.

As regards the second module, i.e., an Fc part of an antibody, the term “Fc part of an antibody” refers to, in its broadest sense, to an “Fc” region which comprises two heavy chain fragments comprising the C_{H}2 and C_{H}3 domains of an antibody. The two heavy chain fragments are held together by two or more disulfide bonds and by hydrophobic interactions of the C_{H}3 domains.

Any Fc-region or Fc-part of an antibody may be used when fused to an antigen correlated with said infectious disease or malignant disease in terms of the present invention as long as said Fc part is capable of binding a receptor present on or in an antigen presenting cell (APC) selected from the group consisting of: Type I: activatory FcγRI, FcγRIIa, FcγRIIc, FcγRIIIa, FcγRIIIb, and inhibitory FcγRIIb; and Type II: neonatal FcR (FcRn) and cytosolic TRIM21.

Assays for assessing the capability of an Fc part of an antibody to bind to a receptor present on or in an antigen presenting cell (APC) are known in the art.

Without being bound to theory, the capability of an Fc part of an antibody to bind to a receptor present on or in an antigen presenting cell (APC) may be assessed as followed:

A fusion protein consisting of the FcR extracellular domain coupled to the intracellular signaling domain of the murine T-cell receptor (CD3zeta) is overexpressed in murine BW5147 cells. Interaction of Fc parts of antibodies or within immune complexes with the extracellular FcR domain induces signaling via the CD3zeta domain and secretion of IL-2 into the culture supernatant. IL-2 in the supernatant can be measured using an ELISA, in which anti-IL-2 antibodies are coated on a plate and IL-2 in the supernatant binds to these antibodies and can be detected using a secondary antibody coupled to an enzyme, commonly horse-reddish peroxidase. After addition of a substrate a color change can be measured using a photometer. The detailed method is described in [56]. Capability of binding to TRIM21 can be measured in vitro using SEC-MALS as described in the art, e.g., in Mallery, et al., 2010 [61]. Cytosolic engagement of TRIM21 can be measured using the TRIMaway assay described in the art (Clift, et al., 2017 [62]) and by comparison of cells in which TRIM21 expression has been knocked-down or knocked-out with cells in which TRIM21 is expressed with respect to the fate of the antigen bound by antibodies, as described for antibodies interacting with Adenovirus and degradation of adenoviral proteins after incubation of cells expressing TRIM21, but not in cells lacking TRIM21, with Adenovirus-antibody complexes; see, e.g., Mallery, et al., 2010 [61].

In preferred embodiments, the Fc-part of an antibody is capable of binding to a receptor present on or in an APC selected from the group consisting of: Type I: activatory FcγRI, FcγRIIa, FcγRIIc, FcγRIIIa, FcγRIIIb, and inhibitory FcγRIIb; and Type II: neonatal FcR (FcRn) and cytosolic TRIM21.

In preferred embodiments, the receptor present on or in an APC may have the following amino acid sequence selected from the group consisting of:
amino acid sequence of an activatory FcγRI transcript variant 1 to 9 (SEQ ID NO:17 to 25); amino acid sequence of an FcγRIIa (SEQ ID NO:26 to 29);
amino acid sequence of an FcγRIIc (SEQ ID NO:30);
amino acid sequence of an FcγRIIIa (SEQ ID NO:31 to 34);
amino acid sequence of an FcγRIIIb (SEQ ID NO:35 to 38);
amino acid sequence of an inhibitory FcγRIIb (SEQ ID NO:39 to 50);
amino acid sequence of an neonatal FcR (FcRn) (SEQ ID NO:51); and
amino acid sequence of an cytosolic TRIM21 (SEQ ID NO:52).

The receptor present on or in an APC to which the Fc-part of the above antibody is capable of binding to is not particularly limited to the amino acid sequences of any one of SEQ ID NOs: 17 to 52 but may also be a receptor sequence which comprises or consists of the amino acid sequences of any one of SEQ ID NOs: 17 to 52 with at least 95%, 90%, 85%, 75%, 70%, 65%, 60%, 55% or 50% sequence homology, preferably identity with the sequences of any one of SEQ ID NOs: 17 to 52 as long as an Fc-part of the antibody has the capability of binding to said receptor present on or in an APC as described herein above and below.

Regarding the determination whether an Fc-part of the antibody has the capability of binding to said receptor present on or in an APC, the same applies as has been set forth above. Corresponding assays for determining this capability has also been set forth above which applies, mutatis mutandis, to the determination whether an Fc-part of the antibody has the capability of binding to any of the above described receptors present on or in an APC.

In order to determine whether an amino acid sequence has a certain degree of identity to the sequences of any one of SEQ ID Nos: 17 to 52, the skilled person can use means and methods well known in the art, e.g. alignments, either manually or by using computer programs known to the person skilled in the art. Such an alignment can, e.g., be done with means and methods known to the skilled person, e.g. by using a known computer algorithm such as the Lipman-Pearson method (Science 227 (1985), 1435) or the CLUSTAL algorithm. It is preferred that in such an alignment maximum homology is assigned to conserved amino acid residues present in the amino acid sequences. In a preferred embodiment ClustalW2 is used for the comparison of amino acid sequences. In the case of pairwise comparisons/alignments, the following settings are preferably chosen: Protein weight matrix: BLOSUM 62; gap open: 10; gap extension: 0.1. In the case of multiple comparisons/alignments, the following settings are preferably chosen: Protein weight matrix: BLOSUM 62; gap open: 10; gap extension: 0.2; gap distance: 5; no end gap.

In accordance with the present invention, the term “identical” or “percent identity” in the context of two or more nucleic acid or amino acid sequences, refers to two or more sequences or subsequences that are the same, or that have a specified percentage of amino acid residues or nucleotides that are the same (e.g., 60% or 65% identity, preferably, 70-95% identity, more preferably at least 95% identity with the nucleic acid sequences or with the amino acid sequences as described above, when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably, the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art.

Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul, (1997) Nucl. Acids Res. 25:3389-3402; Altschul (1993) J. Mol. Evol. 36:290-300; Altschul (1990) J. Mol. Biol. 215:403-410). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff (1989) PNAS 89:10915) uses alignments (B) of 50, expectation (E) of 10, M=5, N=4, and a comparison of both strands.

Preferably, the amino acid substitution(s) are “conservative substitution(s)” which refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity (see, e.g., Watson Molecular Biology of the Gene, The Benjamin/Cummings Pub. Co. 4th Ed. (1987), 224. In addition, substitutions of structurally or functionally similar amino acids are less likely to disrupt biological activity. Within the context of the present invention the binding compounds/antibodies of the present invention comprise polypeptide chains with sequences that include up to 0 (no changes), 1, 2, 3, 4, 5, 6, 7, 8, 9, 10, 12, 15, 20 or more conservative amino acid substitutions when compared with the specific amino acid sequences disclosed herein, for example, SEQ ID NO: 9 (referring to the variable region of the antibody heavy chain of the antibody) and 10 (referring to the variable of the light chain of the antibody). As used herein, the phrase “up to X” conservative amino acid substitutions includes 0 substitutions and any number of substitutions up to 10 and including 0, 1, 2, 3, 4, 5, 6, 7, 8, 9, 10 substitutions.

Such exemplary substitutions are preferably made in accordance with those set forth in Table 1 as follows:

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

As outlined above, the Fc part of the fusion protein is capable of binding a receptor present on or in an antigen presenting cell (APC) as just defined in the preceding embodiment.

Without being bound to theory, in preferred embodiments, the Fc part of the fusion protein may be an Fc domain which is known in the art to bind to one or more of the above receptors.

In further preferred embodiments, the Fc part of the fusion protein may be an Fc domain which has an amino acid sequence selected from the group consisting of:
HDIT101 Fc (SEQ ID NO: 53);
HDIT101 Fc G236A (SEQ ID NO: 54) (in SEQ ID NO:54, the Fc region's mutation G236A is located at position G16A);
HDIT101 Fc G236A/S239D/I332E (SEQ ID NO: 55) (in SEQ ID NO:55, the Fc region's mutation G236A/S239D/I332E is located at position G16A/S19D/I112E, respectively).

Thus, in preferred embodiments, the Fc-part of the Fc-containing fusion protein contains an Fc-part of an antibody wherein said Fc-part of an antibody preferably is/or has the following amino acid sequence selected from the group consisting of: SEQ ID NO: 53 to 55.

The Fc-part of the Fc-containing fusion protein not particularly limited to the amino acid sequences of any one of SEQ ID NOs: 53 to 55 but may also be an Fc-part of an antibody of SEQ ID NOs: 53 to 55 with at least 95%, 90%, 85%, 75%, 70%, 65%, 60%, 55% or 50% sequence homology, preferably, identity with the sequences of any one of SEQ ID NOs: 53 to 55 as long as said Fc-part of the fusion protein has the capability of binding to an Fc receptor present on or in an APC as described herein above and below.

Regarding the determination whether an Fc-part of the fusion has the capability of binding to an receptor present on or in an APC, the same applies as has been set forth above. Corresponding assays for determining this capability has also been set forth above which applies, mutatis mutandis, to the determination whether any of the above-described Fc-parts of the fusion protein has the capability of binding to an receptors present on or in an APC.

As regards the determination of sequence identity, the same applies as has been set forth above.

In a further preferred embodiment, the Fc-part of the Fc-containing fusion protein has an amino acid sequence as shown in SEQ ID NO:54 or an amino acid sequence with at least 95%, 90%, 85%, 75%, 70%, 65%, 60%, 55% or 50% sequence homology, preferably, identity with the sequences of any one of SEQ ID NO: 54, in which an amino acid residue at position 16 in the amino acid sequence shown in SEQ ID NO:54 or at a position corresponding to this position, is substituted with another amino acid residue and wherein said Fc-part of the fusion protein has the capability of binding to an Fc receptor present on or in an APC as described herein above and below. Preferably, an amino acid residue at position 16 in the amino acid sequence shown in SEQ ID NO:54 or at a position corresponding to this position is substituted with the amino acid A.

In a further preferred embodiment, the Fc-part of the Fc-containing fusion protein has an amino acid sequence as shown in SEQ ID NO:55 or an amino acid sequence with at least 95%, 90%, 85%, 75%, 70%, 65%, 60%, 55% or 50% sequence homology, preferably, identity with the sequences of any one of SEQ ID NO: 55, in which an amino acid residue at position 16, 19, and/or 112 in the amino acid sequence shown in SEQ ID NO:55 or at a position corresponding to this position, is substituted with another amino acid residue and wherein said Fc-part of the antibody has the capability of binding to an Fc receptor present on or in an APC as described herein above and below. Preferably, an amino acid residue at position 16 in the amino acid sequence shown in SEQ ID NO:55 or at a position corresponding to this position is substituted with the amino acid A. Preferably, an amino acid residue at position 19 in the amino acid sequence shown in SEQ ID NO:55 or at a position corresponding to this position is substituted with the amino acid D. Preferably, an amino acid residue at position 112 in the amino acid sequence shown in SEQ ID NO:55 or at a position corresponding to this position is substituted with the amino acid E.

In further preferred embodiments, the Fc-containing fusion protein may have an amino acid sequence selected from the group consisting of:
HSV-1 gB extracellular domain fused to HDIT101 Fc domain (SEQ ID NO: 56);
HSV-1 gB extracellular domain fused to HDIT101 Fc domain with G236A (SEQ ID NO: 57) (in SEQ ID NO:57, the Fc region's mutation G236A is located at position G724A);
HSV-1 gB extracellular domain fused to HDIT101 Fc domain with G236A/S239D/I332E (SEQ ID NO: 58) (in SEQ ID NO:58, the Fc region's mutation G236A/S239D/I332E is located at position G724A/S727D/I820E, respectively);
HSV-2 gB extracellular domain fused to HDIT101 Fc domain (SEQ ID NO: 59);
HSV-2 gB extracellular domain fused to HDIT101 Fc domain with G236A (SEQ ID NO: 60) (in
SEQ ID NO:60, the Fc region's mutation G236A is located at position G726A); and
HSV-2 gB extracellular domain fused to HDIT101 Fc domain with G236A/S239D/I332E (SEQ ID NO: 61) (in SEQ ID NO:61, the Fc region's mutation G236A/S239D/I332E is located at position G726A/S729D/I822E, respectively).

Thus, in preferred embodiments, the Fc-containing fusion protein has the following amino acid sequence selected from the group consisting of: SEQ ID NO: 56 to 61.

The Fc-containing fusion protein is not particularly limited to the amino acid sequences of any one of SEQ ID NOs: 56 to 61 but may also be an Fc-containing fusion protein of SEQ ID NOs: 56 to 61 with at least 95%, 90%, 85%, 75%, 70%, 65%, 60%, 55% or 50% sequence homology, preferably, identity with the sequences of any one of SEQ ID NOs: 56 to 51 as long as said Fc-part of the Fc fusion protein has the capability of binding to an Fc receptor present on or in an APC as described herein above and below.

Regarding the determination whether an Fc-part of the fusion protein has the capability of binding to a receptor present on or in an APC, the same applies as has been set forth above. Corresponding assays for determining this capability has also been set forth above which applies, mutatis mutandis, to the determination whether any of the above-described Fc-parts of the fusion protein has the capability of binding to an receptor present on or in an APC.

As regards the determination of sequence identity, the same applies as has been set forth above.

In a further preferred embodiment, the Fc-containing fusion protein has an amino acid sequence as shown in SEQ ID NO:57 or an amino acid sequence with at least 95%, 90%, 85%, 75%, 70%, 65%, 60%, 55% or 50% sequence homology, preferably, identity with the sequences of any one of SEQ ID NO: 57, in which an amino acid residue at position 724 in the amino acid sequence shown in SEQ ID NO:57 or at a position corresponding to this position, is substituted with another amino acid residue and wherein said Fc-part of the fusion protein has the capability of binding to an Fc receptor present on or in an APC as described herein above and below. Preferably, an amino acid residue at position 724 in the amino acid sequence shown in SEQ ID NO:57 or at a position corresponding to this position is substituted with the amino acid A.

In a further preferred embodiment, the Fc-containing fusion protein has an amino acid sequence as shown in SEQ ID NO:58 or an amino acid sequence with at least 95%, 90%, 85%, 75%, 70%, 65%, 60%, 55% or 50% sequence homology, preferably, identity with the sequences of any one of SEQ ID NO: 58, in which an amino acid residue at position 724, 727, and/or 820 in the amino acid sequence shown in SEQ ID NO:58 or at a position corresponding to this position, is substituted with another amino acid residue and wherein said Fc-part of the fusion protein has the capability of binding to an Fc receptor present on or in an APC as described herein above and below. Preferably, an amino acid residue at position 724 in the amino acid sequence shown in SEQ ID NO:58 or at a position corresponding to this position is substituted with the amino acid A. Preferably, an amino acid residue at position 727 in the amino acid sequence shown in SEQ ID NO:58 or at a position corresponding to this position is substituted with the amino acid D. Preferably, an amino acid residue at position 820 in the amino acid sequence shown in SEQ ID NO:58 or at a position corresponding to this position is substituted with the amino acid E.

In a further preferred embodiment, the Fc-containing fusion protein has an amino acid sequence as shown in SEQ ID NO:60 or an amino acid sequence with at least 95%, 90%, 85%, 75%, 70%, 65%, 60%, 55% or 50% sequence homology, preferably, identity with the sequences of any one of SEQ ID NO: 60, in which an amino acid residue at position 726 in the amino acid sequence shown in SEQ ID NO:60 or at a position corresponding to this position, is substituted with another amino acid residue and wherein said Fc-part of the fusion protein has the capability of binding to an Fc receptor present on or in an APC as described herein above and below. Preferably, an amino acid residue at position 726 in the amino acid sequence shown in SEQ ID NO:60 or at a position corresponding to this position is substituted with the amino acid A.

In a further preferred embodiment, the Fc-containing fusion protein has an amino acid sequence as shown in SEQ ID NO:61 or an amino acid sequence with at least 95%, 90%, 85%, 75%, 70%, 65%, 60%, 55% or 50% sequence homology, preferably, identity with the sequences of any one of SEQ ID NO: 61, in which an amino acid residue at position 726, 729, and/or 822 in the amino acid sequence shown in SEQ ID NO:61 or at a position corresponding to this position, is substituted with another amino acid residue and wherein said Fc-part of the fusion protein has the capability of binding to an Fc receptor present on or in an APC as described herein above and below. Preferably, an amino acid residue at position 726 in the amino acid sequence shown in SEQ ID NO:61 or at a position corresponding to this position is substituted with the amino acid A. Preferably, an amino acid residue at position 729 in the amino acid sequence shown in SEQ ID NO:61 or at a position corresponding to this position is substituted with the amino acid D. Preferably, an amino acid residue at position 822 in the amino acid sequence shown in SEQ ID NO:61 or at a position corresponding to this position is substituted with the amino acid E.

Regarding the determination whether an Fc-part of the fusion protein has the capability of binding to an receptor present on or in an APC, the same applies as has been set forth above. Corresponding assays for determining this capability has also been set forth above which applies, mutatis mutandis, to the determination whether any of the above-described Fc-parts of the fusion protein has the capability of binding to an receptors present on or in an APC.

As regards the determination of sequence identity, the same applies as has been set forth above.

The addition of cell penetrating peptides has been shown to induce better MHC-I presentation. Two of the known peptides to induce this are Tat from HIV-1 and the PTD-4 peptide (Del Gaizo, et al., 2003 [63]). Hence, in preferred embodiments, the addition of one or more protein transduction peptides (preferably, Tat peptide and/or PTD-4 peptide) to an Ag-Fc fusion protein as defined herein above is envisaged to further enhance MHC-I presentation.

Thus, in preferred embodiments, the Fc-containing fusion protein has the following amino acid sequence:
HSV-1 gB extracellular domain fused to Tat peptide and HDIT101 Fc domain (SEQ ID NO: 62). The Fc-containing fusion protein is not particularly limited to the amino acid sequence of SEQ ID NO: 62 but may also be an Fc-containing fusion protein of SEQ ID NO: 62 with at least 95%, 90%, 85%, 75%, 70%, 65%, 60%, 55% or 50% sequence homology, preferably, identity with the sequences of SEQ ID NO: 62 as long as said Fc-part of the Fc fusion protein has the capability of binding to an Fc receptor present on or in an APC as described herein above and below.

In further preferred embodiments, the Fc-containing fusion protein has the following amino acid sequence:
HSV-1 gB extracellular domain fused to Tat peptide and HDIT101 Fc domain with G236A (SEQ ID NO: 63) (in SEQ ID NO:63, the Fc region's mutation G236A is located at position G733A). In a further preferred embodiment, the Fc-containing fusion protein has an amino acid sequence as shown in SEQ ID NO:63 or an amino acid sequence with at least 95%, 90%, 85%, 75%, 70%, 65%, 60%, 55% or 50% sequence homology, preferably, identity with the sequences of any one of SEQ ID NO: 63, in which an amino acid residue at position 733 in the amino acid sequence shown in SEQ ID NO:63 or at a position corresponding to this position, is substituted with another amino acid residue and wherein said Fc-part of the fusion protein has the capability of binding to an Fc receptor present on or in an APC as described herein above and below. Preferably, an amino acid residue at position in the amino acid sequence shown in SEQ ID NO:63 or at a position corresponding to this position is substituted with the amino acid A. In further preferred embodiments, the Fc-containing fusion protein has the following amino acid sequence:
HSV-1 gB extracellular domain fused to Tat peptide and HDIT101 Fc domain with G236A/S239D/I332E (SEQ ID NO: 64) (in SEQ ID NO:64, the Fc region's mutation G236A/S239D/I332E is located at position G733A/S736D/I829E, respectively).

In a further preferred embodiment, the Fc-containing fusion protein has an amino acid sequence as shown in SEQ ID NO:64 or an amino acid sequence with at least 95%, 90%, 85%, 75%, 70%, 65%, 60%, 55% or 50% sequence homology, preferably, identity with the sequences of any one of SEQ ID NO: 64, in which an amino acid residue at position 733, 736, and/or 829 in the amino acid sequence shown in SEQ ID NO:64 or at a position corresponding to this position, is substituted with another amino acid residue and wherein said Fc-part of the fusion protein has the capability of binding to an Fc receptor present on or in an APC as described herein above and below. Preferably, an amino acid residue at position 733 in the amino acid sequence shown in SEQ ID NO:64 or at a position corresponding to this position is substituted with the amino acid A. Preferably, an amino acid residue at position 736 in the amino acid sequence shown in SEQ ID NO:64 or at a position corresponding to this position is substituted with the amino acid D. Preferably, an amino acid residue at position 829 in the amino acid sequence shown in SEQ ID NO:64 or at a position corresponding to this position is substituted with the amino acid E.

In further preferred embodiments, the Fc-containing fusion protein has the following amino acid sequence:
HSV-2 gB extracellular domain fused to Tat peptide and HDIT101 Fc domain (SEQ ID NO: 65). The Fc-containing fusion protein is not particularly limited to the amino acid sequence of SEQ ID NO: 65 but may also be an Fc-containing fusion protein of SEQ ID NO: 65 with at least 95%, 90%, 85%, 75%, 70%, 65%, 60%, 55% or 50% sequence homology, preferably, identity with the sequences of SEQ ID NO: 65 as long as said Fc-part of the Fc fusion protein has the capability of binding to an Fc receptor present on or in an APC as described herein above and below.

In further preferred embodiments, the Fc-containing fusion protein has the following amino acid sequence:
HSV-2 gB extracellular domain fused to Tat peptide and HDIT101 Fc domain with G236A (SEQ ID NO: 66) (in SEQ ID NO:66, the Fc region's mutation G236A is located at position G735A).

In a further preferred embodiment, the Fc-containing fusion protein has an amino acid sequence as shown in SEQ ID NO:66 or an amino acid sequence with at least 95%, 90%, 85%, 75%, 70%, 65%, 60%, 55% or 50% sequence homology, preferably, identity with the sequences of any one of SEQ ID NO: 66, in which an amino acid residue at position 735 in the amino acid sequence shown in SEQ ID NO:66 or at a position corresponding to this position, is substituted with another amino acid residue and wherein said Fc-part of the fusion protein has the capability of binding to an Fc receptor present on or in an APC as described herein above and below. Preferably, an amino acid residue at position 735 in the amino acid sequence shown in SEQ ID NO:66 or at a position corresponding to this position is substituted with the amino acid A.

In further preferred embodiments, the Fc-containing fusion protein has the following amino acid sequence:
HSV-2 gB extracellular domain fused to Tat peptide and HDIT101 Fc domain with G236A/S239D/I332E (SEQ ID NO: 67) (in SEQ ID NO:67, the Fc region's mutation G236A/S239D/I332E is located at position G735A/S738D/I831E, respectively).

In a further preferred embodiment, the Fc-containing fusion protein has an amino acid sequence as shown in SEQ ID NO:67 or an amino acid sequence with at least 95%, 90%, 85%, 75%, 70%, 65%, 60%, 55% or 50% sequence homology, preferably, identity with the sequences of any one of SEQ ID NO: 67, in which an amino acid residue at position 735, 738, and/or 831 in the amino acid sequence shown in SEQ ID NO:67 or at a position corresponding to this position, is substituted with another amino acid residue and wherein said Fc-part of the fusion protein has the capability of binding to an Fc receptor present on or in an APC as described herein above and below. Preferably, an amino acid residue at position 735 in the amino acid sequence shown in SEQ ID NO:67 or at a position corresponding to this position is substituted with the amino acid A. Preferably, an amino acid residue at position 738 in the amino acid sequence shown in SEQ ID NO:67 or at a position corresponding to this position is substituted with the amino acid D. Preferably, an amino acid residue at position 831 in the amino acid sequence shown in SEQ ID NO:67 or at a position corresponding to this position is substituted with the amino acid E.

In further preferred embodiments, the Fc-containing fusion protein has the following amino acid sequence:
HSV-1 gB extracellular domain fused to PTD-4 peptide and HDIT101 Fc domain (SEQ ID NO: 68).

The Fc-containing fusion protein is not particularly limited to the amino acid sequence of SEQ ID NO: 68 but may also be an Fc-containing fusion protein of SEQ ID NO: 68 with at least 95%, 90%, 85%, 75%, 70%, 65%, 60%, 55% or 50% sequence homology, preferably, identity with the sequences of SEQ ID NO: 68 as long as said Fc-part of the Fc fusion protein has the capability of binding to an Fc receptor present on or in an APC as described herein above and below.

In further preferred embodiments, the Fc-containing fusion protein has the following amino acid sequence:
HSV-1 gB extracellular domain fused to PTD-4 peptide and HDIT101 Fc domain with G236A (SEQ ID NO: 69) (in SEQ ID NO:69, the Fc region's mutation G236A is located at position G732A).

In a further preferred embodiment, the Fc-containing fusion protein has an amino acid sequence as shown in SEQ ID NO:69 or an amino acid sequence with at least 95%, 90%, 85%, 75%, 70%, 65%, 60%, 55% or 50% sequence homology, preferably, identity with the sequences of any one of SEQ ID NO: 69, in which an amino acid residue at position 732 in the amino acid sequence shown in SEQ ID NO:69 or at a position corresponding to this position, is substituted with another amino acid residue and wherein said Fc-part of the fusion protein has the capability of binding to an Fc receptor present on or in an APC as described herein above and below. Preferably, an amino acid residue at position 732 in the amino acid sequence shown in SEQ ID NO:69 or at a position corresponding to this position is substituted with the amino acid A.

In further preferred embodiments, the Fc-containing fusion protein has the following amino acid sequence:
HSV-1 gB extracellular domain fused to PTD-4 peptide and HDIT101 Fc domain with G236A/S239D/I332E (SEQ ID NO: 70) (in SEQ ID NO:70, the Fc region's mutation G236A/S239D/I332E is located at position G732A/S735D/I828E, respectively).

In a further preferred embodiment, the Fc-containing fusion protein has an amino acid sequence as shown in SEQ ID NO:70 or an amino acid sequence with at least 95%, 90%, 85%, 75%, 70%, 65%, 60%, 55% or 50% sequence homology, preferably, identity with the sequences of any one of SEQ ID NO: 70, in which an amino acid residue at position 732, 735, and/or 828 in the amino acid sequence shown in SEQ ID NO:70 or at a position corresponding to this position, is substituted with another amino acid residue and wherein said Fc-part of the fusion protein has the capability of binding to an Fc receptor present on or in an APC as described herein above and below. Preferably, an amino acid residue at position 732 in the amino acid sequence shown in SEQ ID NO:70 or at a position corresponding to this position is substituted with the amino acid A. Preferably, an amino acid residue at position 735 in the amino acid sequence shown in SEQ ID NO:70 or at a position corresponding to this position is substituted with the amino acid D. Preferably, an amino acid residue at position 828 in the amino acid sequence shown in SEQ ID NO:70 or at a position corresponding to this position is substituted with the amino acid E.

In further preferred embodiments, the Fc-containing fusion protein has the following amino acid sequence:
HSV-2 gB extracellular domain fused to PTD-4 peptide and HDIT101 Fc domain (SEQ ID NO: 71).

The Fc-containing fusion protein is not particularly limited to the amino acid sequence of SEQ ID NO: 71 but may also be an Fc-containing fusion protein of SEQ ID NO: 71 with at least 95%, 90%, 85%, 75%, 70%, 65%, 60%, 55% or 50% sequence homology, preferably, identity with the sequences of SEQ ID NO: 71 as long as said Fc-part of the Fc fusion protein has the capability of binding to an Fc receptor present on or in an APC as described herein above and below.

In further preferred embodiments, the Fc-containing fusion protein has the following amino acid sequence:
HSV-2 gB extracellular domain fused to PTD-4 peptide and HDIT101 Fc domain with G236A (SEQ ID NO: 72) (in SEQ ID NO:72, the Fc region's mutation G236A is located at position G734A).

In a further preferred embodiment, the Fc-containing fusion protein has an amino acid sequence as shown in SEQ ID NO:72 or an amino acid sequence with at least 95%, 90%, 85%, 75%, 70%, 65%, 60%, 55% or 50% sequence homology, preferably, identity with the sequences of any one of SEQ ID NO: 72, in which an amino acid residue at position 734 in the amino acid sequence shown in SEQ ID NO:72 or at a position corresponding to this position, is substituted with another amino acid residue and wherein said Fc-part of the fusion protein has the capability of binding to an Fc receptor present on or in an APC as described herein above and below. Preferably, an amino acid residue at position 734 in the amino acid sequence shown in SEQ ID NO:72 or at a position corresponding to this position is substituted with the amino acid A.

In further preferred embodiments, the Fc-containing fusion protein has the following amino acid sequence:
HSV-2 gB extracellular domain fused to PTD-4 peptide and HDIT101 Fc domain with G236A/S239D/I332E (SEQ ID NO: 73) (in SEQ ID NO:73, the Fc region's mutation G236A/S239D/I332E is located at position G734A/S737D/I830E, respectively).

In a further preferred embodiment, the Fc-containing fusion protein has an amino acid sequence as shown in SEQ ID NO:73 or an amino acid sequence with at least 95%, 90%, 85%, 75%, 70%, 65%, 60%, 55% or 50% sequence homology, preferably, identity with the sequences of any one of SEQ ID NO: 73, in which an amino acid residue at position 734, 737, and/or 830 in the amino acid sequence shown in SEQ ID NO:73 or at a position corresponding to this position, is substituted with another amino acid residue and wherein said Fc-part of the fusion protein has the capability of binding to an Fc receptor present on or in an APC as described herein above and below. Preferably, an amino acid residue at position 734 in the amino acid sequence shown in SEQ ID NO:73 or at a position corresponding to this position is substituted with the amino acid A. Preferably, an amino acid residue at position 737 in the amino acid sequence shown in SEQ ID NO:73 or at a position corresponding to this position is substituted with the amino acid D. Preferably, an amino acid residue at position 830 in the amino acid sequence shown in SEQ ID NO:73 or at a position corresponding to this position is substituted with the amino acid E.

In further preferred embodiments, the Fc-containing fusion protein has the following amino acid sequence:
HSV-1F gB extracellular domain fused to IgG1 hinge*-CH2-CH3 (SEQ ID NO: 74)
HSV-2G gB extracellular domain fused to IgG1 hinge*-CH2-CH3 (SEQ ID NO: 75)
HSV-1F gB extracellular domain fused to IgG1 hinge*-CH2-CH3-StrepTag (SEQ ID NO: 76)
HSV-2G gB extracellular domain fused to IgG1 hinge*-CH2-CH3-StrepTag (SEQ ID NO: 77)
HSV-1F gB extracellular domain fused to StrepTag (SEQ ID NO: 78)
HSV-2G gB extracellular domain fused to StrepTag (SEQ ID NO: 79)

The strep-tagged sequences have also a thrombin cleavage site before the StrepTag to remove it. This has beneficial properties, in particular, for a more efficient production and purification of said Fc-containing fusion protein.

Thus, in further preferred embodiments, the Fc-containing fusion protein has the following amino acid sequence:
HSV-1F gB extracellular domain fused to IgG1 hinge*-CH2-CH3 (SEQ ID NO: 74).

The Fc-containing fusion protein is not particularly limited to the amino acid sequence of SEQ ID NO: 74 but may also be an Fc-containing fusion protein of SEQ ID NO: 74 with at least 95%, 90%, 85%, 75%, 70%, 65%, 60%, 55% or 50% sequence homology, preferably, identity with the sequences of SEQ ID NO: 74 as long as said Fc-part of the Fc fusion protein has the capability of binding to an Fc receptor present on or in an APC as described herein above and below.

In further preferred embodiments, the Fc-containing fusion protein has the following amino acid sequence:
HSV-2G gB extracellular domain fused to IgG1 hinge*-CH2-CH3 (SEQ ID NO: 75)

The Fc-containing fusion protein is not particularly limited to the amino acid sequence of SEQ ID NO: 75 but may also be an Fc-containing fusion protein of SEQ ID NO: 75 with at least 95%, 90%, 85%, 75%, 70%, 65%, 60%, 55% or 50% sequence homology, preferably, identity with the sequences of SEQ ID NO: 75 as long as said Fc-part of the Fc fusion protein has the capability of binding to an Fc receptor present on or in an APC as described herein above and below.

In further preferred embodiments, the Fc-containing fusion protein has the following amino acid sequence:
HSV-1F gB extracellular domain fused to IgG1 hinge*-CH2-CH3-StrepTag (SEQ ID NO: 76)

The Fc-containing fusion protein is not particularly limited to the amino acid sequence of SEQ ID NO: 76 but may also be an Fc-containing fusion protein of SEQ ID NO: 76 with at least 95%, 90%, 85%, 75%, 70%, 65%, 60%, 55% or 50% sequence homology, preferably, identity with the sequences of SEQ ID NO: 76 as long as said Fc-part of the Fc fusion protein has the capability of binding to an Fc receptor present on or in an APC as described herein above and below.

In further preferred embodiments, the Fc-containing fusion protein has the following amino acid sequence:
HSV-2G gB extracellular domain fused to IgG1 hinge*-CH2-CH3-StrepTag (SEQ ID NO: 77)

The Fc-containing fusion protein is not particularly limited to the amino acid sequence of SEQ ID NO: 77 but may also be an Fc-containing fusion protein of SEQ ID NO: 77 with at least 95%, 90%, 85%, 75%, 70%, 65%, 60%, 55% or 50% sequence homology, preferably, identity with the sequences of SEQ ID NO: 77 as long as said Fc-part of the Fc fusion protein has the capability of binding to an Fc receptor present on or in an APC as described herein above and below.

In further preferred embodiments, there are also disclosed fusion proteins which do not have an Fc-containing part. A corresponding protein has the following amino acid sequence:
HSV-1F gB extracellular domain fused to StrepTag (SEQ ID NO: 78)

This protein is not particularly limited to the amino acid sequence of SEQ ID NO: 78 but may also be a fusion protein of SEQ ID NO: 78 with at least 95%, 90%, 85%, 75%, 70%, 65%, 60%, 55% or 50% sequence homology, preferably, identity with the sequences of SEQ ID NO: 78 as long as said protein has the capability of binding to an Fc receptor present on or in an APC as described herein above and below.

In further preferred embodiments, there is also disclosed a fusion protein which does not have an Fc-containing part. A corresponding has the following amino acid sequence:
HSV-2G gB extracellular domain fused to StrepTag (SEQ ID NO: 79)

This protein is not particularly limited to the amino acid sequence of SEQ ID NO: 79 but may also be a protein of SEQ ID NO: 79 with at least 95%, 90%, 85%, 75%, 70%, 65%, 60%, 55% or 50% sequence homology, preferably, identity with the sequences of SEQ ID NO: 79 as long as said protein has the capability of binding to an Fc receptor present on or in an APC as described herein above and below.

Regarding the determination whether an Fc-part of the fusion protein has the capability of binding to a receptor present on or in an APC, the same applies as has been set forth above.

Corresponding assays for determining this capability has also been set forth above which applies, mutatis mutandis, to the determination whether any of the above-described Fc-parts of the fusion protein has the capability of binding to a receptor present on or in an APC.

As regards the determination of sequence identity, the same applies as has been set forth above.

As mentioned, according to (b), the construct comprising an Fc part of an antibody fused to an antigen correlated with said infectious disease or malignant disease is an “Fc-containing fusion protein”.

Thus, in a preferred embodiment, the construct according to (b) is present in the form of a fusion protein, i.e., a protein which is formed by the expression of a hybrid gene made by combining at least two gene sequences. Typically, as will be explained in more detail further below, this is accomplished by cloning a cDNA into an expression vector in frame with an existing gene. Accordingly, the construct may be a fusion protein, i.e., a chimeric molecule which is formed by joining two or more polypeptides via a peptide bond between the amino terminus of one module and the carboxyl terminus of another molecule. In this way, the above Fc part of an antibody and the antigen correlated with said infectious disease or malignant disease are joined together in the form of a fusion protein wherein both parts are proteinaceous of nature. Once cloned in frame, the fusion protein is then recombinantly expressed by a corresponding nucleic acid sequence encoding said fusion protein.

Alternatively, the Fc part of an antibody and the antigen correlated with said infectious disease or malignant disease may also be covalently coupled by a chemical conjugate. Thus, the modules of the construct may be chemically coupled in a covalent linkage. In particular, in preferred embodiments, multimeric complexes with multiple Fc domains and/or multiple antigen molecules are envisaged. A corresponding example of a multimeric complex is shown in Argentinian AntiCovid Consortium 2022 [64].

Accordingly, in such an embodiment, both parts of (a), i.e., the Fc part of an antibody and the antigen correlated with said infectious disease or malignant disease, may individually be synthesized (either chemically or by recombinant technology), optionally purified and then chemically coupled in a covalent linkage. Thus, the Fc-containing fusion protein of the present invention may be a construct, wherein the Fc part of an antibody and the antigen correlated with said infectious disease or malignant disease are chemically coupled in a covalent linkage. The term “chemically coupled in a covalent linkage” relates to conjugation techniques which are well-known to the skilled person. Many methods for making covalent or non-covalent conjugates with proteins or peptides are known in the art and any such known method may be utilized. Without being bound to theory, a construct according to the present invention can be prepared by using a heterobifunctional cross-linker, such as N-succinyl 3-(2-pyridyldithio)propionate (SPDP). Yu et al., Int. J. Cancer 56: 244 (1994). General techniques for such conjugation are well-known in the art; see, for example, Wong, Chemistry of Protein Conjugation and Cross-linking (CRC Press 1991); Upeslacis et al., “Modification of Antibodies by Chemical Methods,” in Monoclonal Antibodies: Principles and Applications, Birch et al. (eds.), pages 187-230 (Wiley-Liss, Inc. 1995); Price, “Production and Characterization of Synthetic Peptide-Derived Antibodies,” in Monoclonal Antibodies: Production, Engineering and Clinical Application, Ritter et al. (eds.), pages 60-84 (Cambridge University Press 1995). Therefore, in view of the fact that methods for coupling the Fc part of an antibody and the antigen correlated with said infectious disease or malignant disease in a covalent linkage to each other, preferable to a protein/peptide are well-known to the person skilled in the art the examples provided herewith are not limiting.

The Fc-containing fusion protein in accordance with the present invention may not only comprise the above two parts, i.e., the Fc part of an antibody and the antigen correlated with said infectious disease or malignant disease. Rather, it may be desirable that between the individual parts (a) linker moiety/moieties are placed which may, e.g., facilitate the construction of the fusion protein.

Yet, as mentioned above, the Fc-containing fusion protein of the present invention is, in a preferred embodiment, a “classical” fusion protein. A variety of methods are known for making fusion proteins, including nucleic acid synthesis, hybridization and/or amplification to produce a synthetic double-stranded nucleic acid encoding a fusion protein of interest. Such double-stranded nucleic acids may be inserted into expression vectors for fusion protein production by standard molecular biology techniques (see, e.g. Sambrook et al., Molecular Cloning, A laboratory manual, 2nd Ed, 1989).

Accordingly, a nucleic acid molecule may be prepared encoding the Fc-containing fusion protein comprising the Fc part of an antibody and the antigen correlated with said infectious disease or malignant disease as defined above. The nucleic acid is, for example a DNA, encoding the Fc-containing fusion protein comprising the Fc part of an antibody and the antigen correlated with said infectious disease or malignant disease. The above nucleic acid molecule of the present invention may be a natural nucleic acid molecule as well as a recombinant nucleic acid molecule. The nucleic acid molecule of the invention may, therefore, be of natural origin, synthetic or semi-synthetic. It may comprise DNA, RNA as well as PNA and it may be a hybrid thereof.

It is evident to the person skilled in the art that regulatory sequences may be added to the nucleic acid molecule of the invention, in particular for its (recombinant) expression. For example, promoters, transcriptional enhancers and/or sequences which allow for induced expression of the polynucleotide of the invention may be employed. A suitable inducible system is for example tetracycline-regulated gene expression as described, e.g., by Gossen and Bujard, Proc. Natl. Acad. Sci. USA 89 (1992), 5547-5551) and Gossen, Trends Biotech. 12 (1994), 58-62, or a dexamethasone-inducible gene expression system as described, e.g. by Crook, EMBO J. 8 (1989), 513-519.

Furthermore, said nucleic acid molecule may contain, for example, thioester bonds and/or nucleotide analogues. Said modifications may be useful for the stabilization of the nucleic acid molecule against endo- and/or exonucleases in the cell. Said nucleic acid molecules may be transcribed by an appropriate vector containing a chimeric gene which allows for the transcription of said nucleic acid molecule in the cell. In the context of the present invention said nucleic acid molecules are labeled. Methods for the detection of nucleic acids are well known in the art, e.g., Southern and Northern blotting, PCR or primer extension.

The nucleic acid molecule(s) encoding the Fc-containing fusion protein comprising the Fc part of an antibody and the antigen correlated with said infectious disease or malignant disease as described above may be a recombinantly produced chimeric nucleic acid molecule comprising any of the aforementioned nucleic acid molecules either alone or in combination. Preferably, the nucleic acid molecule of the invention is part of a vector.

The vector of the present invention may be, e.g., a plasmid, cosmid, virus, bacteriophage or another vector used e.g. conventionally in genetic engineering, and may comprise further genes such as marker genes which allow for the selection of said vector in a suitable host cell and under suitable conditions.

Furthermore, the vector of the present invention may, in addition to the nucleic acid sequences of the invention, comprise expression control elements, allowing proper expression of the coding regions in suitable hosts. Such control elements are known to the skilled person and may include a promoter, a splice cassette, translation initiation codon, translation and insertion site for introducing an insert into the vector. Preferably, the nucleic acid molecule of the invention is operatively linked to said expression control sequences allowing expression in eukaryotic or prokaryotic cells. Accordingly, the present invention relates to a vector comprising the nucleic acids of the invention, wherein the nucleic acid is operably linked to control sequences that are recognized by a host cell when the eukaryotic and/or prokaryotic (host) cell is transfected with the vector.

Control elements ensuring expression in eukaryotic and prokaryotic (host) cells are well known to those skilled in the art. As mentioned herein above, they usually comprise regulatory sequences ensuring initiation of transcription and optionally poly-A signals ensuring termination of transcription and stabilization of the transcript. Additional regulatory elements may include transcriptional as well as translational enhancers, and/or naturally-associated or heterologous promoter regions. Possible regulatory elements permitting expression in for example mammalian host cells comprise the CMV-HSV thymidine kinase promoter, SV40, RSV-promoter (Rous Sarcoma Virus), human elongation factor 1α-promoter, the glucocorticoid-inducible MMTV-promoter Mouse Mammary Tumor Virus), metallothionein- or tetracyclin-inducible promoters, or enhancers, like CMV enhancer or SV40-enhancer. For expression in neural cells, it is envisaged that neurofilament-, PGDF-, NSE-, PrP-, or thy-1-promoters can be employed. Said promoters are known in the art and, inter alia, described in Charron, J. Biol. Chem. 270 (1995), 25739-25745. For the expression in prokaryotic cells, a multitude of promoters including, for example, the tac-lac-promoter or the trp promoter, has been described. Besides elements which are responsible for the initiation of transcription such regulatory elements may also comprise transcription termination signals, such as SV40-poly-A site or the tk-poly-A site, downstream of the polynucleotide. In this context, suitable expression vectors are known in the art such as Okayama-Berg cDNA expression vector pcDV1 (Pharmacia), pRc/CMV, pcDNA1, pcDNA3 (In-vitrogene), pSPORT1 (GIBCO BRL), pX (Pagano, Science 255 (1992), 1144-1147), yeast two-hybrid vectors, such as pEG202 and dpJG4-5 (Gyuris, Cell 75 (1995), 791-803), or prokaryotic expression vectors, such as lambda gt11 or pGEX (Amersham-Pharmacia). Beside the nucleic acid molecules of the present invention, the vector may further comprise nucleic acid sequences encoding for secretion signals. Such sequences are well known to the person skilled in the art. Furthermore, depending on the expression system used leader sequences capable of directing the peptides of the invention to a cellular compartment may be added to the coding sequence of the nucleic acid molecules of the invention and are well known in the art. The leader sequence(s) is (are) assembled in appropriate phase with translation, initiation and termination sequences, and preferably, a leader sequence capable of directing secretion of translated protein, or a protein thereof, into the periplasmic space or extracellular medium. Optionally, the heterologous sequence can encode a fusion protein including a C- or N-terminal identification peptide imparting desired characteristics, e.g., stabilization or simplified purification of expressed recombinant product. Once the vector has been incorporated into the appropriate host, the host is maintained under conditions suitable for high level expression of the nucleotide sequences, and, as desired, the collection and purification of the antibody molecules or fragments thereof of the invention may follow.

Furthermore, the vector of the present invention may also be an expression vector. The nucleic acid molecules and vectors of the invention may be designed for direct introduction or for introduction via liposomes, viral vectors (e.g. adenoviral, retroviral), electroporation, ballistic (e.g. gene gun) or other delivery systems into the cell. Additionally, a baculoviral system can be used as eukaryotic expression system for the nucleic acid molecules of the invention.

For its expression, a host cell may be prepared comprising the vector of the present invention. Thus, the present invention relates to a host transfected or transformed with the vector of the invention or a non-human host carrying the vector of the present invention, i.e., to a host cell or host which is genetically modified with a nucleic acid molecule according to the invention or with a vector comprising such a nucleic acid molecule. The term “genetically modified” means that the host cell or host comprises in addition to its natural genome a nucleic acid molecule or vector according to the invention which was introduced into the cell or host or into one of its predecessors/parents. The nucleic acid molecule or vector may be present in the genetically modified host cell or host either as an independent molecule outside the genome, preferably as a molecule which is capable of replication, or it may be stably integrated into the genome of the host cell or host. The transformation of the host cell with a vector according to the invention can be carried out by standard methods, as for instance described in Sambrook and Russell (2001), Molecular Cloning: A Laboratory Manual, CSH Press, Cold Spring Harbor, NY, USA; Methods in Yeast Genetics, A Laboratory Course Manual, Cold Spring Harbor Laboratory Press, 1990. The host cell is cultured in nutrient media meeting the requirements of the particular host cell used, in particular in respect of the pH value, temperature, salt concentration, aeration, antibiotics, vitamins, trace elements etc. Corresponding host cells used for the expression of the Fc-containing fusion protein of the present invention may be any prokaryotic or eukaryotic cell. Suitable prokaryotic cells are those generally used for cloning like E. coli or Bacillus subtilis. Furthermore, eukaryotic cells comprise, for example, fungal or animal cells. Examples for suitable fungal cells are yeast cells, preferably those of the genus Saccharomyces and most preferably those of the species Saccharomyces cerevisiae. Suitable animal cells are, for instance, insect cells, vertebrate cells, preferably mammalian cells, such as e.g. HEK293, NSO, CHO, COS-7, MDCK, U2-OSHela, NIH3T3, MOLT-4, Jurkat, PC-12, PC-3, IMR, NT2N, Sk-n-sh, CaSki, C33A. These host cells, e.g. CHO-cells, may provide posts-translational (secondary) modifications to the antibody molecules of the invention, including leader peptide removal, folding and assembly of H and C chains, glycosylation of the molecule at correct sides and secretion of the functional molecule. Further suitable cell lines known in the art are obtainable from cell line depositories, like, e.g., the Deutsche Sammlung von Mikroorganismen und Zellkulturen GmbH (DSMZ) or the American Type Culture Collection (ATCC). In accordance with the present invention, it is furthermore envisaged that primary cells/cell cultures may function as host cells. Said cells are in particular derived from insects (like insects of the species Drosophila or Blatta) or mammals (like human, swine, mouse or rat). Said host cells may also comprise cells from and/or derived from cell lines like neuroblastoma cell lines. The above mentioned primary cells are well known in the art and comprise, inter alia, primary astrocytes, (mixed) spinal cultures or hippocampal cultures.

The Fc-containing fusion protein comprising the Fc part of an antibody and the antigen correlated with said infectious disease or malignant disease may be produced by methods known in the art. As an example, the above host cell harbouring an expression vector encoding the Fc-containing fusion protein comprising the Fc part of an antibody and the antigen correlated with said infectious disease or malignant disease of the invention may be cultured in culture medium. The Fc-containing fusion protein comprising the Fc part of an antibody and the antigen correlated with said infectious disease or malignant disease may then be recovered from the host cell or culture medium.

Host cells, e.g., CHO cells, may provide post-translational (secondary) modification on the expressed binding compounds of the present invention. These modifications comprise, inter alia, glycosylation and phosphorylation.

Methods of recovering and/or subsequently purifying the construct of the present invention are known to the person skilled in the art.

In a preferred embodiment, the vaccine for use according to the present invention as described herein above, said active immunization elicits a persistent or long-term immunity against said infectious disease or malignant disease in said subject and wherein said subject suffers from an acute infectious disease or a malignant disease.

Preferably, a persistent or long-term immunity, in an active immunization, has effects for several months to years. In case of eradication of e.g., tumors or cancer cells the term “long-term”, in a preferred embodiment, relates to an effect that is “life-long”. For chronic recurrent infectious diseases, e.g., in preferred embodiments, HSV infection the term “long-term” is the protection of reactivation of symptom-free months to years.

In preferred embodiments of the first aspect of the present invention, in the vaccine for use as defined above, the antibody or the Fc-containing fusion protein is capable of eliciting antibody dependent cellular phagocytosis (ADCP) independent from antibody-dependent cellular cytotoxicity (ADCC) and/or complement-dependent cytotoxicity (CDC). In more preferred embodiments, in the vaccine for use as defined above, the Fc-containing fusion protein is capable of eliciting antibody dependent cellular phagocytosis (ADCP) independent from antibody-dependent cellular cytotoxicity (ADCC) and/or complement-dependent cytotoxicity (CDC) while the Fc-containing fusion protein may be capable of mediating ADCC and CDC in case the Fc-containing fusion protein comprising an Fc part of an antibody fused to an antigen is able to bridge to cells via binding of said antigen to cells, so that Fc is free and can mediate ADCC or CDC.

In the following, the second aspect, is described in more detail.

As mentioned above, in a second aspect, which is related to the above first aspect, the present invention relates to a vaccine for use in actively immunizing a subject against an infectious disease, wherein said infectious disease is an HSV-associated disease; and
(a) wherein said antibody is an anti-HSV antibody and said subject suffers from an acute HSV-associated disease, preferably an acute HSV infection; or
(b) wherein an Fc-containing fusion protein comprising an Fc part of an antibody is fused to an antigen, wherein the antigen correlates with an HSV-associated disease, wherein said wherein said Fc part is capable of binding a receptor present on or in an antigen presenting cell (APC) selected from the group consisting of:
   Type I: activatory FcγRI, FcγRIIa, FcγRIIc, FcγRIIIa, FcγRIIIb, and inhibitory FcγRIIb; and
   Type II: neonatal FcR (FcRn) and cytosolic TRIM21.

Thus, the vaccine of the second aspect of the present invention may comprise one of the two alternative components (while, it is also contemplated that the vaccine comprises both components), i.e.,
(a) wherein said antibody is an anti-HSV antibody and said subject suffers from an acute HSV-associated disease, preferably an acute HSV infection; or
(b) wherein an Fc-containing fusion protein comprising an Fc part of an antibody is fused to an antigen, wherein the antigen correlates with an HSV-associated disease, wherein said wherein said Fc part is capable of binding a receptor present on or in an antigen presenting cell (APC) selected from the group consisting of:
   Type I: activatory FcγRI, FcγRIIa, FcγRIIc, FcγRIIIa, FcγRIIIb, and inhibitory FcγRIIb; and
   Type II: neonatal FcR (FcRn) and cytosolic TRIM21.

These two components are separately described in more detail in the following.

An Anti-HSV Antibody and Said Subject Suffers from an Acute HSV-Associated Disease, Preferably an Acute HSV Infection:

As regards (a), i.e., the antibody being an anti-HSV antibody and said subject suffering from an acute HSV-associated disease, preferably an acute HSV infection, the same applies, mutatis mutandis, as has been set forth above in relation to the first aspect of the present invention with the exception that the “antibody against an antigen correlated with said infectious disease” is an anti-HSV antibody and said “antigen correlated with said infectious disease” is an antigen that correlates with an acute HSV-associated disease, preferably an acute HSV infection.

As mentioned above, the antibody contains at least an Fc-part of an antibody in line with the rationale of the present invention.

The antibody as used in the vaccine of the second aspect of the present invention is not particularly limited as long as it is an “anti-HSV antibody”. Thus, the antibody may be any antibody which specifically binds to or specifically recognizes or interacts with a HSV, i.e., a domain or an antigen of a HSV.

The term “binding to” as used in the context of the present invention defines a binding (interaction) of at least two “antigen-interaction-sites” with each other. The term “antigen-interaction-site” defines, in accordance with the present invention, a motif of a polypeptide, i.e., a part of the antibody or antigen-binding fragment of the present invention, which shows the capacity of specific interaction with a specific antigen or a specific group of antigens of the HSV. Said binding/interaction is also understood to define a “specific recognition”. The term “specifically recognizing” means in accordance with this invention that the antibody is capable of specifically interacting with and/or binding to at least two amino acids of each of a HSV as defined herein. Antibodies can recognize, interact and/or bind to different epitopes on a HSV. This term relates to the specificity of the antibody molecule, i.e., to its ability to discriminate between the specific regions of a HSV.

The term “specific interaction” as used in accordance with the present invention means that the antibody or antigen-binding fragment thereof of the invention does not or does not essentially cross-react with (poly) peptides of similar structures. Accordingly, the antibody or antigen-binding fragment thereof of the invention specifically binds to/interacts with structures of a HSV, preferably HSV-1 or HSV-2. Specific examples of such molecules against which said first and second, Ig-derived domain is directed are given herein below.

Cross-reactivity of a panel of antibody or antigen-binding fragment thereof under investigation may be tested, for example, by assessing binding of said panel of antibody or antigen-binding fragment thereof under conventional conditions (see, e.g., Harlow and Lane, Antibodies: A Laboratory Manual, Cold Spring Harbor Laboratory Press, (1988) and Using Antibodies: A Laboratory Manual, Cold Spring Harbor Laboratory Press, (1999)) to the (poly)peptide of interest as well as to a number of more or less (structurally and/or functionally) closely related (poly)peptides. Only those constructs (i.e. antibodies, antigen-binding fragments thereof and the like) that bind to the certain structure of the HSV, e.g., a specific epitope or (poly) peptide/protein of the HSV but do not or do not essentially bind to any of the other epitope or (poly) peptides of the same HSV, are considered specific for the epitope or (poly) peptide/protein of interest and selected for further studies in accordance with the method provided herein. These methods may comprise, inter alia, binding studies, blocking and competition studies with structurally and/or functionally closely related molecules. These binding studies also comprise FACS analysis, surface plasmon resonance (SPR, e.g. with BIAcore®), analytical ultracentrifugation, isothermal titration calorimetry, fluorescence anisotropy, fluorescence spectroscopy or by radiolabeled ligand binding assays.

The term “binding to” does not only relate to a linear epitope but may also relate to a conformational epitope, a structural epitope or a discontinuous epitope consisting of two regions of the human target molecules or parts thereof. In the context of this invention, a conformational epitope is defined by two or more discrete amino acid sequences separated in the primary sequence which comes together on the surface of the molecule when the polypeptide folds to the native protein (Sela, Science 166 (1969), 1365 and Laver, Cell 61 (1990), 553-536). Moreover, the term “binding to” is interchangeably used in the context of the present invention with the terms “interacting with” or “recognizing”.

Accordingly, specificity can be determined experimentally by methods known in the art and methods as described herein. Such methods comprise, but are not limited to Western Blots, ELISA-, RIA-, ECL-, IRMA-tests and peptide scans.

As mentioned above, the vaccine for use in actively immunizing a subject against an infectious disease (wherein said infectious disease is an HSV-associated disease and (a) wherein said antibody is an anti-HSV antibody and said subject suffers from an acute HSV-associated disease, preferably an acute HSV infection) requires the presence of an acute infection. This is because, for being able actively immunizing the subject in line with the rationale of the present invention, not only the antibody but also an antigen correlated with said HSV infection must be present.

“Acute” in this respect means that the subject has a breakout of the disease and, accordingly, HSV is present in the body. The breakout of the disease can either follow a primary infection or follow a reactivation (outbreak) from the HSV's latent reservoir.

In fact, it is well known that after a primary infection HSV spreads from infected epithelial cells to axons of sensory neurons innervating the site of the primary infection followed by retrograde transport to the respective dorsal root ganglia, where HSV establishes a latent reservoir for life. HSV infection of neurons exists as a reversible state and episodes of viral reactivation (outbreaks) may occur from time to time. Reactivation of the virus can be triggered by a wide range of stress stimuli (e.g. immunodeficiency, trauma, fever, menstruation, UV light and sexual intercourse) that act on the neuron, or at a peripheral site innervated by the infected ganglion, or systemically. Intermittent HSV reactivations result in the production of infectious HSV from latently infected neurons. Once reactivated the virus is transported by the neuron back to the nerve terminals in the epithelium.

In a more preferred embodiment, “acute” means that the subject shows symptoms of the disease. In other words, the subject to be vaccinated is in actual need of a vaccination or treatment and the term “acute” relates to situations after the (primary) onset or the reactivation breakout of the disease. The term “acute” as referred to in the context of the present invention is opposed to a prophylactic or preventive, i.e., measures taken for disease prevention, e.g., in order to prevent the infection and/or the onset/outbreak of the disease.

More specifically, prophylactic treatment may be understood in a way that it prevents attachment of free virus particles (from outside the body) to target cells and in turn prevents virus replication. In contrast, at an acute infection (which could be a primary or a recurrent infection) progeny virus have been raced upon HSV replication.

As will be outlined in more detail further below, the vaccine for use according to the second aspect of the present invention is for use in actively immunizing a subject against an HSV-associated disease, wherein said disease is selected from the group consisting of Herpes simplex labialis, Herpes simplex genitalis, chronic or disseminated cutaneous herpes simplex infection, Herpes gladiatorum, Eczema herpeticum, Herpes keratoconjunctivitis, Herpes neonatorum, Alzheimer disease (AD), HSV pneumonia, Bell's palsy, Herpes esophagitis, Herpesviral encephalitis and Herpesviral meningitis, Herpetic sycosis, Herpes withlow, Herpes gingivostomatitis, presence of an oral recidive, presence of a genital recidive, eczema herpeticatum, herpes neonatorum, immune deficiency, immunocompromised patients, resistance against a virusstatic agent, encephalitis, meningitis, meningoencephalitis, eye infections, and/or generalized HSV infections.

In more preferred embodiments, the vaccine for use according to the second aspect of the present invention is for use in actively immunizing a subject against an HSV-associated disease, wherein said disease is caused by HSV-1 or HSV-2, even more preferably wherein said HSV-associated disease is selected from the group consisting of Herpes simplex labialis, Herpes simplex genitalis, chronic or disseminated cutaneous herpes simplex infection, Herpes gladiatorum and Eczema herpeticum is to be topically administered.

In a preferred embodiment, the anti-HSV antibody of the vaccine for use according the second aspect of the present invention is a monoclonal. In a further preferred embodiment, the anti-HSV antibody of the vaccine for use according the second aspect of the present invention is a humanized or a fully human antibody. In a further preferred embodiment, the anti-HSV antibody of the vaccine for use according the second aspect of the present invention is a murine antibody.

As already outlined above in the context of the first aspect of the present invention, the term “monoclonal antibody” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Monoclonal antibodies are advantageous in that they may be synthesized by a hybridoma culture, essentially uncontaminated by other immunoglobulins. The modified “monoclonal” indicates the character of the antibody as being amongst a substantially homogeneous population of antibodies, and is not to be construed as requiring production of the antibody by any particular method. As mentioned above, the monoclonal antibodies to be used in accordance with the present invention may be made by the hybridoma method described by Kohler, Nature 256 (1975), 495.

The term “polyclonal antibody” as used herein in the context of the present disclosure, refers to an antibody which was produced among or in the presence of one or more other, non-identical antibodies. In general, polyclonal antibodies are produced from a B-lymphocyte in the presence of several other B-lymphocytes which produced non-identical antibodies. Usually, polyclonal antibodies are obtained directly from an immunized animal.

The term “fully-human antibody” as used herein refers to an antibody which comprises human immunoglobulin protein sequences only. A fully human antibody may contain murine carbohydrate chains if produced in a mouse, in a mouse cell or in a hybridoma derived from a mouse cell. Similarly, “mouse antibody” or “murine antibody” refers to an antibody which comprises mouse/murine immunoglobulin protein sequences only. Alternatively, a “fully-human antibody” may contain rat carbohydrate chains if produced in a rat, in a rat cell, in a hybridoma derived from a rat cell. Similarly, the term “rat antibody” refers to an antibody that comprises rat immunoglobulin sequences only. Fully-human antibodies may also be produced, for example, by phage display which is a widely used screening technology which enables production and screening of fully human antibodies. Also phage antibodies can be used in context of this invention. Phage display methods are described, for example, in U.S. Pat. Nos. 5,403,484, 5,969,108 and 5,885,793. Another technology which enables development of fully-human antibodies involves a modification of mouse hybridoma technology. Mice are made transgenic to contain the human immunoglobulin locus in exchange for their own mouse genes (see, for example, U.S. Pat. No. 5,877,397).

The term “chimeric antibodies”, refers to an antibody which comprises a variable region of the present invention fused or chimerized with an antibody region (e.g., constant region) from another, human or non-human species (e.g., mouse, horse, rabbit, dog, cow, chicken).

The term antibody also relates to recombinant human antibodies, heterologous antibodies and heterohybrid antibodies. The term “recombinant human antibody” includes all human sequence antibodies that are prepared, expressed, created or isolated by recombinant means, such as antibodies isolated from an animal (e.g., a mouse) that is transgenic for human immunoglobulin genes; antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences. Such recombinant human antibodies have variable and constant regions (if present) derived from human germline immunoglobulin sequences. Such antibodies can, however, be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

A “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

The term “heterohybrid antibody” refers to an antibody having light and heavy chains of different organismal origins. For example, an antibody having a human heavy chain associated with a murine light chain is a heterohybrid antibody. Examples of heterohybrid antibodies include chimeric and humanized antibodies.

The term antibody also relates to humanized antibodies. “Humanized” forms of non-human (e.g. murine or rabbit) antibodies are chimeric immunoglobulins, immunoglobulin chains which contain minimal sequence derived from non-human immunoglobulin. Often, humanized antibodies are human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Furthermore, humanized antibody may comprise residues, which are found neither in the recipient antibody nor in the imported CDR or framework sequences. These modifications are made to further refine and optimize antibody performance. In general, the humanized antibody will comprise substantially all of at least one, and typically two variable domains, in which all or substantially all of the CDR regions correspond to those of a non-human immunoglobulin and all or substantially all of the FR regions are those of a human immunoglobulin consensus sequence. The humanized antibody may also comprise at least a portion of an immunoglobulin constant region (Fc), typically that of a human immunoglobulin. For further details, see: Jones Nature 321 (1986), 522-525; Reichmann Nature 332 (1998), 323-327 and Presta Curr Op Struct Biol 2 (1992), 593-596.

As already outlined above in the context of the first aspect of the present invention, a popular method for humanization of antibodies involves CDR grafting, where a functional antigen-binding site from a non-human ‘donor’ antibody is grafted onto a human ‘acceptor’ antibody. CDR grafting methods are known in the art and described, for example, in U.S. Pat. Nos. 5,225,539, 5,693,761 and 6,407,213. Another related method is the production of humanized antibodies from transgenic animals that are genetically engineered to contain one or more humanized immunoglobulin loci which are capable of undergoing gene rearrangement and gene conversion (see, for example, U.S. Pat. No. 7,129,084).

Accordingly, in context of the present invention, the term “antibody” relates to full immunoglobulin molecules as well as to parts of such immunoglobulin molecules (i.e., “antigen-binding fragment thereof”). Furthermore, the term relates, in general terms, to modified and/or altered antibody molecules. The term also relates to recombinantly or synthetically generated/synthesized antibodies. The term also relates to intact antibodies. The term antibody also comprises but is not limited to fully-human antibodies, chimeric antibodies, humanized antibodies, CDR-grafted antibodies and antibody constructs, like single chain Fvs (scFv) or antibody-fusion proteins.

In a preferred embodiment, the anti-HSV antibody of the vaccine for use according the second aspect of the present invention is a full-length antibody, i.e., a full immunoglobulin molecule which is often also referred to as complete antibody.

“Single-chain Fv” or “scFv” antibody fragments (which are not envisaged in the context of the second aspect of the present invention) have, in the context of the invention, the V_{H} and V_{L} domains of an antibody, wherein these domains are present in a single polypeptide chain. Generally, the scFv polypeptide further comprises a polypeptide linker between the V_{H} and V_{L} domains which enables the scFv to form the desired structure for antigen binding. Techniques described for the production of single chain antibodies are described, e.g., in Pluckthun in The Pharmacology of Monoclonal Antibodies, Rosenburg and Moore eds. Springer-Verlag, N.Y. (1994), 269-315.

A “Fab fragment” as used herein is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}2 and C_{H}3 domains of an antibody. The two heavy chain fragments are held together by two or more disulfide bonds and by hydrophobic interactions of the C_{H}3 domains.

A “Fab′ fragment” contains one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

Antibodies, antibody constructs, antibody fragments, antibody derivatives (all being Ig-derived) to be employed in accordance with the invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook (1989), loc. cit. The term “Ig-derived domain” particularly relates to (poly) peptide constructs comprising at least one CDR. Fragments or derivatives of the recited Ig-derived domains define (poly) peptides which are parts of the above antibody molecules and/or which are modified by chemical/biochemical or molecular biological methods. Corresponding methods are known in the art and described inter alia in laboratory manuals (see Sambrook et al., Molecular Cloning: A Laboratory Manual; Cold Spring Harbor Laboratory Press, 2nd edition (1989) and 3rd edition (2001); Gerhardt et al., Methods for General and Molecular Bacteriology ASM Press (1994); Lefkovits, Immunology Methods Manual: The Comprehensive Sourcebook of Techniques; Academic Press (1997); Golemis, Protein-Protein Interactions: A Molecular Cloning Manual Cold Spring Harbor Laboratory Press (2002)).

The antibody of the vaccine for use according the second aspect of the present invention is not particularly limited as long as it is an “anti-HSV antibody”. Thus, the antibody may be any antibody which specifically binds to or specifically recognizes or interacts with a HSV, i.e., a domain, an antigen, preferably a surface-antigen of a HSV. The skilled person is readily in a position to generate such an antibody directed to a given domain (i.e., an antigen, preferably a surface-antigen of a HSV) and determine whether a respective antibody is capable of detecting/binding to a given domain, an antigen, preferably a surface-antigen of a HSV, preferably HSV-1 and/or HSV-2 based on the skilled person's common general knowledge and the methods described above.

In a preferred embodiment, the antibody of the vaccine for use according the second aspect of the present invention binds to/recognizes the viral antigen glycoproteins D, B, C, H, L, E or I (i.e., gD, gB, gC, gH, gL, gE, gI) Glycoproteins D, B, C, H, L, E and I are surface or envelope proteins of HSV-1 and/or HSV-2. These proteins may not only be found on the surface or in the envelope structure of HSV-1 and/or HSV-2, i.e., on the surface of released infectious particles (i.e., the envelope of free virions) but they may also be present on the surface of infected cells, i.e., on the surface of cells. Yet, in a more preferred embodiment, the antibody of the invention binds to/recognizes the viral surface antigen glycoprotein D, B, C, H, L, E or I (i.e., gD, gB, gC, gH, gL, gE, or gI) of the HSV-1 and/or HSV-2 envelope. In a preferred embodiment, the anti-HSV antibody or the antigen-binding fragment thereof for use according to the present invention recognizes the surface glycoprotein B (gB) of the HSV-1 and/or HSV-2 envelope, preferably an epitope thereof. The glycoprotein B of HSV-1 and/or HSV-2 is well-characterized and, without being bound to specific sequences, examples sequences of various HSV-1 and HSV-2 strains, respectively, are shown in SEQ ID NOs:11 to 16. SEQ ID NO:11 shows the sequence of the glycoprotein B of HSV-1 strain F, SEQ ID NO:12 shows the sequence of the glycoprotein B of HSV-1 strain KOS, SEQ ID NO:13 shows the sequence of the glycoprotein B of HSV-1 strain gC-39-R6, SEQ ID NO:14 shows the sequence of the glycoprotein B of HSV-2 strain HG52, SEQ ID NO:15 shows the sequence of the glycoprotein B of HSV-2 strain 333 and SEQ ID NO:16 shows the sequence of the glycoprotein B of HSV-2 strain MMA. A sequence alignment of these glycoprotein B amino acid sequences shows that the overall amino acid homology, preferably, identity of gB of HSV-1 and HSV-2 is 85% while the sequences are least conserved at the N- and C-terminal regions of the protein.

In a preferred embodiment, the anti-HSV antibody of the vaccine for use according the second aspect of the present invention, is capable of inhibiting the spreading of HSV from an infected cell to an adjacent second non-infected cell (cell-to-cell spread).

Cell-to-cell spread is the ability of the herpes virus to spread to an adjacent second non-infected cell without releasing cell-free particles. Reducing or eliminating the ability of the herpes virus to spread to an adjacent cell has the beneficial effect that the generation of lesions is avoided. In order to examine whether an antibody is capable of inhibiting the spread of HSV from an infected cell to an adjacent second non-infected cell (cell-to-cell spread), methods well-known to the person skilled in the art can be used. As an example, the following assay can be used: Vero cells grown to confluency on glass cover slips in 24-well tissue culture plates are infected for 4 h at 37° C. with a constant virus amount of 400 TCID₅₀/well. One median tissue culture infective dose (1 TCID₅₀) is the mount of a cytopathogenic agent, such as a virus, that will produce a cytopathic effect in 50% of the cell cultures inoculated. The virus inoculum is subsequently removed, the cells washed twice with PBS and further incubated for 2 days at 37° C. in 1 ml DMEM, 2% FCS, Pen/Strep containing an excess of either different anti-HSV antibodies or polyclonal anti-HSV control serum in order to prevent viral spreading via the supernatant. Viral antigens of HSV-infected cells are detected with a fluorescence labelled polyclonal goat-anti-HSV-serum (BETHYL Laboratories, Montgomery, TX USA, Catalog No. A190-136F, Lot No. A190-136F-2). Preferably, an antibody is inhibiting cell-to-cell spread if less than 20% of the adjacent cells are infected, preferably wherein less than 15%, less than 10%, less than 5%, more preferably less than 3% and most preferably less than 1% of the adjacent cells are infected in the above assay.

Cell-to-cell spread may also be assayed as follows: The presence of neutralizing antibodies does not necessarily prevent cell-to-cell spread of herpesviridae. To compare antibodies on disruption of HSV-1 and HSV-2 cell-to-cell spread this particular dissemination mode can be mimicked in vitro using standard test methods. E.g.: To infect individual cells, confluent Vero cell monolayers are initially incubated with either HSV-1 or HSV-2 at low MOI (e.g. 100 TCID50), respectively. After 4 h of adsorption at 37° C., the viral inoculum has to be removed. To promote direct cell-to-cell transmission from individually infected cells but prevent viral spread through viral particles across the cell culture supernatant, Vero cell monolayers are treated with an excess of neutralizing anti-gB antibodies, controls, or medium alone. After 48 h virus spread can be detected by immunostaining with a mouse monoclonal antibody specific for a common epitope on glycoprotein D of HSV-1 and HSV-2 (e.g. Acris Antibodies, San Diego, CA, USA) and fluorescence-conjugated secondary antibody. Immunofluorescence images can be acquired with a fluorescence microscope at a 100- or 400-fold magnification.

Moreover, only disclosed in the context of the present invention, the anti-HSV antibody of the vaccine for use according the second aspect of the present invention may also be capable of neutralizing HSV. “Neutralizing” herein means that the antibody opsonizes the virus so that it cannot infect any further cell. An assay for testing whether an antibody in a concentration of, e.g., at most 20 nM is capable of neutralizing a defined amount of HSV of, e.g., 100 TCID₅₀ Eis-Hübinger et al., Intervirology 32:351-360 (1991); Eis-Hübinger et al., Journal of General Virology 74:379-385 (1993) and in Examples 1 and 2 of WO2011/038933 A2. Thus, in a preferred embodiment, the antibody of the invention in a concentration of at most 20 nM, preferably of at most 16 nM, more preferably of at most 12 nM, such as of at most 10 nm, e.g., at most 8 nM or at most 6 nM, and most preferably of at most 4 nM is capable of neutralizing a defined amount of HSV of 100 TCID₅₀ to more than 80%, preferably by more than 90%, such as more than 95%, more preferably 96%, e.g., more than 97%, and most preferably more than 98%, e.g., more than 99% or even 100%.

Moreover, only disclosed in the context of the present invention, the anti-HSV antibody of the vaccine for use according the second aspect of the present invention may also be capable of inhibiting cell-to-cell spread independent from antibody-dependent cellular cytotoxicity (ADCC) and/or complement-dependent cytotoxicity (CDC).

As the above-described assays for testing the capability whether an antibody is capable of inhibiting cell-to-cell spread do not contain complement and/or cytotoxic effector cells, the same assays may be used in order to determine whether an antibody is capable of inhibiting cell-to-cell spread independent from antibody-dependent cellular cytotoxicity (ADCC) and/or complement-dependent cytotoxicity (CDC).

In a preferred embodiment, the anti-HSV antibody of the vaccine for use according the second aspect of the present invention comprises the complementarity determining regions V_{H}CDR1 comprising SEQ ID NO: 1, V_{H}CDR2 comprising SEQ ID NO: 2, V_{H}CDR3 comprising SEQ ID NO: 3, V_{L}CDR1 comprising SEQ ID NO: 4, V_{L}CDR2 comprising SEQ ID NO: 5, and V_{L}CDR3 comprising SEQ ID NO:6.

The term “CDR” as employed herein relates to “complementary determining region”, which is well known in the art. The CDRs are parts of immunoglobulins that determine the specificity of said molecules and make contact with a specific ligand. The CDRs are the most variable part of the molecule and contribute to the diversity of these molecules. There are three CDR regions CDR1, CDR2 and CDR3 in each V domain. CDR-H depicts a CDR region of a variable heavy chain and CDR-L relates to a CDR region of a variable light chain. VH means the variable heavy chain and VL means the variable light chain. The CDR regions of an Ig-derived region may be determined as described in Kabat “Sequences of Proteins of Immunological Interest”, 5th edit. NIH Publication no. 91-3242 U.S. Department of Health and Human Services (1991); Chothia J. Mol. Biol. 196 (1987), 901-917 or Chothia Nature 342 (1989), 877-883.

Accordingly, in the context of the present invention, the antibody molecule described herein above is selected from the group consisting of a full antibody (immunoglobulin, like an IgG1, an IgG2, an IgG2a, an IgG2b, an IgA1, an IgGA2, an IgG3, an IgG4, an IgA, an IgM, an IgD or an IgE), F(ab)-, Fab′-SH-, Fv-, Fab′-, F(ab′)2-fragment), a chimeric antibody, a CDR-grafted antibody, a fully human antibody, a bivalent antibody-construct, an antibody-fusion protein, a synthetic antibody, bivalent single chain antibody, a trivalent single chain antibody and a multivalent single chain antibody.

“Humanization approaches” are well known in the art and in particular described for antibody molecules, e.g. Ig-derived molecules. The term “humanized” refers to humanized forms of non-human (e.g., murine) antibodies or fragments thereof (such as Fv, Fab, Fab′, F(ab′), scFvs, or other antigen-binding partial sequences of antibodies) which contain some portion of the sequence derived from non-human antibody. Humanized antibodies include human immunoglobulins in which residues from a complementary determining region (CDR) of the human immunoglobulin are replaced by residues from a CDR of a non-human species such as mouse, rat or rabbit having the desired binding specificity, affinity and capacity. In general, the humanized antibody will comprise substantially all of at least one, and generally two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin; see, inter alia, Jones et al., Nature 321 (1986), 522-525, Presta, Curr. Op. Struct. Biol. 2 (1992), 593-596. Methods for humanizing non-human antibodies are well known in the art. Generally, a humanized antibody has one or more amino acids introduced into it from a source which is non-human still retain the original binding activity of the antibody. Methods for humanization of antibodies/antibody molecules are further detailed in Jones et al., Nature 321 (1986), 522-525; Reichmann et al., Nature 332 (1988), 323-327; and Verhoeyen et al., Science 239 (1988), 1534-1536. Specific examples of humanized antibodies, e.g. antibodies directed against EpCAM, are known in the art, see e.g. (LoBuglio, Proceedings of the American Society of Clinical Oncology Abstract (1997), 1562 and Khor, Proceedings of the American Society of Clinical Oncology Abstract (1997), 847).

Accordingly, in the context of this invention, antibody molecules are provided, which are humanized and can successfully be employed in pharmaceutical compositions.

Moreover, in a preferred embodiment, the antibody of the present invention is an antibody that binds to the glycoprotein B (gB) of HSV-1 and/or HSV-2 which comprises or consists of VH domain (heavy chain variable region) and VL domain (light chain variable region), i.e., the amino acid sequence of the variable region of the heavy chain of an antibody as depicted in SEQ ID NO:9 and the amino acid sequence of the variable region of the light chain of an antibody as depicted in SEQ ID NO:10.

However, the antibody as used in the present invention is not particularly limited to such variable heavy and light chain variable regions but may also be an antibody or antigen-binding fragment thereof that binds to the glycoprotein B (gB) of HSV-1 and/or HSV-2 envelope which comprises or consists of VH domain and VL domain with at least 95%, 90%, 85%, 75%, 70%, 65%, 60%, 55% or 50% sequence homology, preferably identity with the sequences of SEQ ID NOs: 9 and 10, respectively, as long as the antibody has the capability of having an effect in actively immunizing a subject against an HSV-associated disease as described herein above and below. Furthermore, the antibody or antigen-binding fragment thereof is a molecule that comprises VH and VL domains having up to 0, 1, 2, 3, 4, 5, 6, 7, 8, 9, 10 or more conservative amino acid substitutions with reference to the sequences of SEQ ID NOs: 9 and 10. Moreover, the antibody or antigen-binding fragment thereof is an antibody fragment selected from the group consisting of Fab, Fab′, Fab′-SH, FV, scFV, F(ab′)2, and a diabody.

In order to determine whether an amino acid sequence has a certain degree of identity to the sequences of SEQ ID NOs: 9 and 10, the skilled person can use means and methods well known in the art, e.g. alignments, either manually or by using computer programs known to the person skilled in the art. Such an alignment can, e.g., be done with means and methods known to the skilled person, e.g. by using a known computer algorithm such as the Lipman-Pearson method (Science 227 (1985), 1435) or the CLUSTAL algorithm. It is preferred that in such an alignment maximum homology is assigned to conserved amino acid residues present in the amino acid sequences. In a preferred embodiment ClustalW2 is used for the comparison of amino acid sequences. In the case of pairwise comparisons/alignments, the following settings are preferably chosen: Protein weight matrix: BLOSUM 62; gap open: 10; gap extension: 0.1. In the case of multiple comparisons/alignments, the following settings are preferably chosen: Protein weight matrix: BLOSUM 62; gap open: 10; gap extension: 0.2; gap distance: 5; no end gap.

In accordance with the present invention, the term “identical” or “percent identity” in the context of two or more nucleic acid or amino acid sequences, refers to two or more sequences or subsequences that are the same, or that have a specified percentage of amino acid residues or nucleotides that are the same (e.g., 60% or 65% identity, preferably, 70-95% identity, more preferably at least 95% identity with the nucleic acid sequences or with the amino acid sequences as described above which are capable of binding to gB of HSV-1 or HSV-2 and having the capability of having an effect in actively immunizing a subject against an HSV-associated disease as described herein above and below and/or being capable of inhibiting the spreading of HSV from an infected cell to an adjacent second non-infected cell (cell-to-cell spread) or being capable of inhibiting cell-to-cell spread independent from antibody-dependent cellular cytotoxicity (ADCC) and/or complement-dependent cytotoxicity (CDC) as described herein above and below), when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably, the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art.

Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul, (1997) Nucl. Acids Res. 25:3389-3402; Altschul (1993) J. Mol. Evol. 36:290-300; Altschul (1990) J. Mol. Biol. 215:403-410). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff (1989) PNAS 89:10915) uses alignments (B) of 50, expectation (E) of 10, M=5, N=4, and a comparison of both strands.

Preferably, the amino acid substitution(s) are “conservative substitution(s)” which refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity (see, e.g., Watson Molecular Biology of the Gene, The Benjamin/Cummings Pub. Co. 4th Ed. (1987), 224. In addition, substitutions of structurally or functionally similar amino acids are less likely to disrupt biological activity. Within the context of the present invention the binding compounds/antibodies of the present invention comprise polypeptide chains with sequences that include up to 0 (no changes), 1, 2, 3, 4, 5, 6, 7, 8, 9, 10, 12, 15, 20 or more conservative amino acid substitutions when compared with the specific amino acid sequences disclosed herein, for example, SEQ ID NO: 9 (referring to the variable region of the antibody heavy chain of the antibody) and 10 (referring to the variable of the light chain of the antibody). As used herein, the phrase “up to X” conservative amino acid substitutions includes 0 substitutions and any number of substitutions up to 10 and including 0, 1, 2, 3, 4, 5, 6, 7, 8, 9, 10 substitutions.

Such exemplary substitutions are preferably made in accordance with those set forth in Table 1 as follows:

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

Moreover, in a preferred embodiment, the anti-HSV antibody of the vaccine for use according to the second aspect of the present invention comprises an amino acid sequence with at least 70% sequence identity to the amino acid residues shown in positions 1 to 30, 38 to 51, 68 to 99, and 112 to 122 of SEQ ID NO: 7 and in positions 1 to 23, 41 to 55, 63 to 94, and 104 to 114 of SEQ ID NO: 8.

In a further, preferred embodiment, the anti-HSV antibody of the vaccine for use according to the second aspect of the present invention comprises an amino acid sequence with at least 75%, at least 80%, more preferably at least 85%, at least 90%, even more preferably at least 95%, and most preferably 98% overall sequence identity in the framework regions compared to the amino acid residues shown in positions 1 to 30, 38 to 51, 68 to 99, and 112 to 122 of SEQ ID NO: 7 and in positions 1 to 23, 41 to 55, 63 to 94, and 104 to 114 of SEQ ID NO: 8. Such antibodies are suitable for the vaccination of the present invention as long as the antibody or antigen-binding fragment binds to gB of HSV-1 or HSV-2 and has the capability of having an effect in actively immunizing a subject against an HSV-associated disease as described herein above and below and/or being capable of inhibiting the spreading of HSV from an infected cell to an adjacent second non-infected cell (cell-to-cell spread) and/or being capable of inhibiting cell-to-cell spread independent from antibody-dependent cellular cytotoxicity (ADCC) and/or complement-dependent cytotoxicity (CDC) as described herein above and below.

Thus, in a preferred embodiment, the anti-HSV antibody of the vaccine for use according to the second aspect of the present invention comprises an amino acid sequence having the above variable regions of the light and heavy chains (i.e., the CDRs defined above, i.e., V_{H}CDR1 comprising SEQ ID NO: 1, V_{H}CDR2 comprising SEQ ID NO: 2, V_{H}CDR3 comprising SEQ ID NO: 3, V_{L}CDR1 comprising SEQ ID NO: 4, V_{L}CDR2 comprising SEQ ID NO: 5, and V_{L}CDR3 comprising SEQ ID NO:6) while the amino acid sequence have a variability in the framework region with at least 75%, at least 80%, more preferably at least 85%, at least 90%, even more preferably at least 95%, and most preferably 98% overall sequence identity in the framework regions compared to the amino acid residues shown in positions 1 to 30, 38 to 51, 68 to 99, and 112 to 122 of SEQ ID NO: 7 and in positions 1 to 23, 41 to 55, 63 to 94, and 104 to 114 of SEQ ID NO: 8.

In this context, a polypeptide has “at least X % sequence identity” in the framework regions to SEQ ID NO:7 or 8 if SEQ ID NO:7 or SEQ ID NO: 8 is aligned with the best matching sequence of a polypeptide of interest and the amino acid identity between those two aligned sequences is at least X % over positions 1 to 30, 38 to 51, 68 to 99, and 112 to 122 of SEQ ID NO: 7 and positions 1 to 23, 41 to 55, 63 to 94, and 104 to 114 of SEQ ID NO: 8. As mentioned above, such an alignment of amino acid sequences can be performed using, for example, publicly available computer homology programs such as the “BLAST” program provided on the National Centre for Biotechnology Information (NCBI) homepage using default settings provided therein. Further methods of calculating sequence identity percentages of sets of amino acid sequences or nucleic acid sequences are known in the art.

Moreover, in a preferred embodiment, the anti-HSV antibody of the vaccine for use according to the second aspect of the present invention comprises the V_{H} of SEQ ID NO:9 and the V_{L} of SEQ ID NO:10.

The sequence of the glycoprotein B of HSV-1 and/or HSV-2 is well-characterized and, as defined above, without being bound to specific sequences, examples sequences of various HSV-1 and HSV-2 strains, respectively, are shown in SEQ ID NOs:11 to 16. The epitope recognized by the mAb 2c antibody is highly conserved among various HSV-strains as well as between HSV-1 and HSV-2.

This antibody of the vaccine for use according to the second aspect of the present invention is not limited to the antibody detecting the above epitope of glycoprotein B of HSV-1 and HSV-2. In fact, also other antibodies which detect another epitope of glycoprotein B or even an epitope of another protein or polypeptide of HSV-1 and HSV-2 can be used in the vaccine actively immunizing a subject against a HSV-associated disease in terms of the present invention and in line with the rationale of the present invention as described herein above and below.

With the normal skill of the person skilled in the art and by routine methods, the person skilled in the art can easily deduce from the sequences provided herein relevant epitopes (also functional fragments) of the polypeptides of HSV which are useful in the generation of antibodies like polyclonal and monoclonal antibodies. However, the person skilled in the art is readily in a position to also provide for engineered antibodies like CDR-grafted antibodies or also humanized and fully human antibodies and the like.

Particularly preferred in the context of the present invention are monoclonal antibodies. For the preparation of monoclonal antibodies, any technique which provides antibodies produced by continuous cell line cultures can be used. Examples for such techniques include the hybridoma technique, the trioma technique, the human B-cell hybridoma technique and the EBV-hybridoma technique to produce human monoclonal antibodies (Shepherd and Dean (2000), Monoclonal Antibodies: A Practical Approach, Oxford University Press, Goding and Goding (1996), Monoclonal Antibodies: Principles and Practice—Production and Application of Monoclonal Antibodies in Cell Biology, Biochemistry and Immunology, Academic Pr Inc, USA).

The antibody derivatives can also be produced by peptidomimetics. Further, techniques described for the production of single chain antibodies (see, inter alia, U.S. Pat. No. 4,946,778) can be adapted to produce single chain antibodies specifically recognizing an antigen of HSV. Also, transgenic animals may be used to express humanized antibodies to the polypeptide of HSV.

Without being bound to theory, an anti-HSV antibody of the vaccine for use according to the second aspect of the present invention can be produced against any native polypeptides and recombinant polypeptides of glycoprotein B or any another protein or polypeptide of HSV-1 and HSV-2. This production is based, for example, on the immunization of animals, like mice. However, also other animals for the production of antibody/antisera are envisaged within the present invention. For example, monoclonal and polyclonal antibodies can be produced by rabbit, mice, goats, donkeys and the like. The polynucleotide encoding a correspondingly chosen polypeptide of HSV-1 or HSV-2 can be subcloned into an appropriated vector, wherein the recombinant polypeptide is to be expressed in an organism being able for an expression, for example in bacteria. Thus, the expressed recombinant protein can be intra-peritoneally injected into a mice and the resulting specific antibody can be, for example, obtained from the mice serum being provided by intra-cardiac blood puncture. The present invention also envisages the production of specific antibodies against native polypeptides and recombinant polypeptides by using a DNA vaccine strategy as exemplified in the appended examples. DNA vaccine strategies are well-known in the art and encompass liposome-mediated delivery, by gene gun or jet injection and intramuscular or intradermal injection. Thus, antibodies directed against a polypeptide or a protein or an epitope of HSV-1 and HSV-2 can be obtained by directly immunizing the animal by directly injecting intramuscularly the vector expressing the desired polypeptide or a protein or an epitope of HSV-1 and HSV-2, in particular an epitope of gB. The amount of obtained specific antibody can be quantified using an ELISA, which is also described herein below. Further methods for the production of antibodies are well known in the art, see, e.g. Harlow and Lane, “Antibodies, A Laboratory Manual”, CSH Press, Cold Spring Harbor, 1988.

The term “specifically binds”, as used herein, refers to a binding reaction that is determinative of the presence of the desired polypeptide or a protein or an epitope of HSV-1 and HSV-2, in particular an epitope of gB, and an antibody in the presence of a heterogeneous population of proteins and other biologics.

Thus, under designated assay conditions, the specified antibodies and a corresponding polypeptide or a protein or an epitope of HSV-1 and HSV-2, in particular an epitope of gB, bind to one another and do not bind in a significant amount to other components present in a sample. Specific binding to a target analyte under such conditions may require a binding moiety that is selected for its specificity for a particular target analyte. A variety of immunoassay formats may be used to select antibodies specifically reactive with a particular antigen. For example, solid-phase ELISA immunoassays are routinely used to select monoclonal antibodies specifically immunoreactive with an analyte. See Shepherd and Dean (2000), Monoclonal Antibodies: A Practical Approach, Oxford University Press and/or Howard and Bethell (2000) Basic Methods in Antibody Production and Characterization, Crc. Pr. Inc. for a description of immunoassay formats and conditions that can be used to determine specific immunoreactivity. Typically a specific or selective reaction will be at least twice background signal to noise and more typically more than 10 to 100 times greater than background. The person skilled in the art is in a position to provide for and generate specific binding molecules directed against the novel polypeptides. For specific binding-assays it can be readily employed to avoid undesired cross-reactivity, for example polyclonal antibodies can easily be purified and selected by known methods (see Shepherd and Dean, loc. cit.).

The term “anti-HSV antibody” means in accordance with this invention that the antibody molecule or antigen-binding fragment thereof is capable of specifically recognizing or specifically interacting with and/or binding to at least two amino acids of the desired polypeptide or a protein or an epitope of HSV-1 and HSV-2, in particular an epitope of gB. Said term relates to the specificity of the antibody molecule, i.e. to its ability to discriminate between the specific regions a desired polypeptide or a protein or an epitope of HSV-1 and HSV-2, in particular an epitope of gB. Accordingly, specificity can be determined experimentally by methods known in the art and methods as disclosed and described herein. Such methods comprise, but are not limited to Western blots, ELISA-, RIA-, ECL-, IRMA-tests and peptide scans. Such methods also comprise the determination of K_{D}-values as, inter alia, illustrated in the appended examples. The peptide scan (pepspot assay) is used routinely employed to map linear epitopes in a polypeptide antigen. The primary sequence of the polypeptide is synthesized successively on activated cellulose with peptides overlapping one another. The recognition of certain peptides by the antibody to be tested for its ability to detect or recognize a specific antigen/epitope is scored by routine colour development (secondary antibody with horseradish peroxide and 4-chloronaphthol and hydrogen peroxide), by a chemoluminescence reaction or similar means known in the art. In the case of, inter alia, chemoluminescence reactions, the reaction can be quantified. If the antibody reacts with a certain set of overlapping peptides one can deduce the minimum sequence of amino acids that are necessary for reaction. The same assay can reveal two distant clusters of reactive peptides, which indicate the recognition of a discontinuous, i.e. conformational epitope in the antigenic polypeptide (Geysen (1986), Mol. Immunol. 23, 709-715).

A preferred epitope of the anti-HSV antibody is defined above and below is the same that is recognized by the mAb2c.

In a preferred embodiment, the anti-HSV antibody of the vaccine for use according to the second aspect of the present invention is the mAb 2c antibody (or an antigen-binding fragment thereof). This monoclonal antibody MAb 2c has been described elsewhere and has been demonstrated to neutralize virus by abrogating viral cell-to-cell spread, a key mechanism by which HSV-1/2 escapes humoral immune surveillance independent from antibody-dependent cellular cytotoxicity (ADCC) and/or complement-dependent cytotoxicity (CDC); Eis-Hübinger et al., Intervirology 32:351-360 (1991); Eis-Hübinger et al., Journal of General Virology 74:379-385 (1993); WO2011/038933 A2; Krawczyk A, et al., Journal of virology (2011); 85(4):1793-1803; Krawczyk A, et al., Proc Natl Acad Sci USA (2013); 110(17):6760-6765.

As outlined above, the vaccine of the second aspect of the present invention may comprise one of the two alternative components (while it is also contemplated that the vaccine comprises both components), i.e.,
(a) wherein said antibody is an anti-HSV antibody and said subject suffers from an acute HSV-associated disease, preferably an acute HSV infection; or
(b) wherein an Fc-containing fusion protein comprising an Fc part of an antibody is fused to an antigen, wherein the antigen correlates with an HSV-associated disease, wherein said wherein said Fc part is capable of binding a receptor present on or in an antigen presenting cell (APC) selected from the group consisting of:
   Type I: activatory FcγRI, FcγRIIa, FcγRIIc, FcγRIIIa, FcγRIIIb, and inhibitory FcγRIIb; and
   Type II: neonatal FcR (FcRn) and cytosolic TRIM21.

The second component (b) in more detail in the following.

An Fc-Containing Fusion Protein Comprising an Fc Part of an Antibody is Fused to an Antigen, Wherein the Antigen Correlates with an HSV-Associated Disease, Wherein Said Fc Part is Capable of Binding a Receptor Present on or in an Antigen Presenting Cell (APC):

As regards (b), i.e., Fc-containing fusion protein comprising an Fc part of an antibody fused to an antigen that correlates with an HSV-associated disease and corresponding preferred embodiments, the same applies, mutatis mutandis, as has been set forth above in relation to the first aspect of the present invention with the exception that the “antigen correlated with said infectious disease” is an antigen that correlates with an acute HSV-associated disease, preferably an acute HSV infection.

As regards said “antigen that correlates with an acute HSV-associated disease, preferably an acute HSV infection” and corresponding preferred embodiments, the same applies, mutatis mutandis, as has been set forth above in relation to the alternative (a) of the second aspect of the present invention.

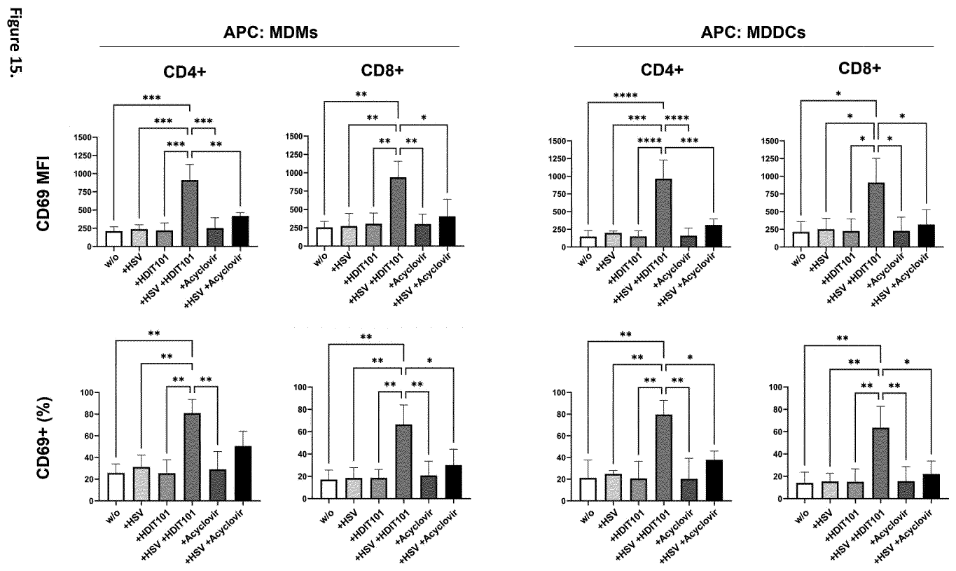

Without being bound to theory and as outlined in Example 2, below, and as illustrated in FIG. 13, further, more preferred embodiments of the above second aspect of the present invention are envisaged.

The rational of an induction of de novo T cell and B cell responses to induce long-lasting protection from HSV lesion recurrences due to the features of the HDIT101 antibody to not induce ADCC or CDC combined with its binding properties to its target protein gB, led to the proposal to combine the effects to generate a vaccine.

Thus, in a preferred embodiment, the vaccine of the present invention may comprise whole virus particles that are complexed with an anti-HSV antibody of the present invention, preferably the HDIT101 antibody.

Moreover, in a preferred embodiment, the vaccine of the present invention may comprise a trimeric gB ectodomain fused to the Fc portion of an anti-HSV antibody, preferably the HDIT101 antibody, complexed in addition with an anti-HSV antibody, preferably the HDIT101 antibody.

In further preferred embodiments, ADCP can even be further enhanced by the introduction of specific amino acid substitutions in Fc (e.g., G236A).

Accordingly, in preferred embodiments, the antibody and the Fc-containing fusion protein comprising an Fc part of an antibody, respectively, of the second aspect of the present invention comprises an Fc part of an antibody which has an amino acid substitution at position 236, 239 and/or 332 in the Fc part of an antibody.

Corresponding preferred embodiments and sequences apply, mutatis mutandis, to the second aspect of the present invention as defined above in the context of the first aspect of the present invention.

Without being bound to theory, as an Fc-fusion protein, gB-Fc adopts trimeric structures that contain 3 Fc domains, two of which dimerize, leaving one free Fc domain that can dimerize with free Fc domains of other gB-Fc trimers generating larger complexes of multiple copies of gB-Fc.

To increase immunogenicity, in further preferred embodiments, to reveal HDIT101-binding induced novel epitopes and to direct the structures towards ADCP, an anti-HSV antibody of the present invention, preferably the HDIT101 antibody, can be contacted with gB-Fc protein units leading to multimeric complexes of gB-Fc protein multivalently bound to an anti-HSV antibody, preferably the HDIT101 antibody. These structures combine the feature of the gB-Fc Ag target and the properties of an anti-HSV antibody in terms of the present invention, preferably the HDIT101 antibody, for enhanced ADCP, while importantly avoiding ADCC and CDC induction, which is proposed to lead to a largely increased T-cell (and B-cell) response and vaccinal effects. In preferred embodiments, a corresponding mAb-enhanced IC can be used as vehicle for enhanced delivery of unrelated Ags into the ADCP pathway, leading to the activation of de novo T-cell (and B-cell) responses.

Accordingly, in preferred embodiments, as it is, e.g., illustrated in FIG. 13, the Fc-containing fusion protein may comprise, in addition to the antigen correlated with an infectious disease or malignant disease (preferably with an HSV-infectious disease), an unrelated antigen fused thereto.

A corresponding “unrelated antigen” may be any antigen as described in the context of the first aspect of the present invention. Thus, as regards corresponding preferred embodiments regarding an “unrelated antigen” of an Fc-containing fusion protein that comprises, in addition to the antigen correlated with an infectious disease or malignant disease (preferably with an HSV-infectious disease) such an unrelated antigen fused thereto, the same applies, mutatis mutandis, as has been set forth above in relation to the first aspect of the present invention.

In preferred embodiments of the second aspect of the present invention, in the vaccine for use as defined above, the antibody or the Fc-containing fusion protein is capable of eliciting antibody dependent cellular phagocytosis (ADCP) independent from antibody-dependent cellular cytotoxicity (ADCC) and/or complement-dependent cytotoxicity (CDC). In more preferred embodiments, in the vaccine for use as defined above, the Fc-containing fusion protein is capable of eliciting antibody dependent cellular phagocytosis (ADCP) independent from antibody-dependent cellular cytotoxicity (ADCC) and/or complement-dependent cytotoxicity (CDC) while the Fc-containing fusion protein may be capable of mediating ADCC and CDC in case the Fc-containing fusion protein comprising an Fc part of an antibody fused to an antigen is able to bridge to cells via binding of said antigen to cells, so that Fc is free and can mediate ADCC or CDC.

In further preferred embodiments, the vaccine of the first and second aspect of the present invention, more specifically, the antibody and the Fc-containing fusion protein, respectively, thereof, is capable of eliciting antibody dependent cellular phagocytosis (ADCP) independent from antibody-dependent cellular cytotoxicity (ADCC) and/or complement-dependent cytotoxicity (CDC).

In more preferred embodiments, in the vaccine for use as defined above, the Fc-containing fusion protein is capable of eliciting antibody dependent cellular phagocytosis (ADCP) independent from antibody-dependent cellular cytotoxicity (ADCC) and/or complement-dependent cytotoxicity (CDC) while the Fc-containing fusion protein may be capable of mediating ADCC and CDC in case the Fc-containing fusion protein comprising an Fc part of an antibody fused to an antigen is able to bridge to cells via binding of said antigen to cells, so that Fc is free and can mediate ADCC or CDC.

“Independent from antibody-dependent cellular cytotoxicity (ADCC) and/or complement-dependent cytotoxicity (CDC)” means that the antibody and the Fc-containing fusion protein, respectively, of the present invention is capable of eliciting antibody dependent cellular phagocytosis (ADCP) without inducing antibody-dependent cellular cytotoxicity (ADCC) and/or complement-dependent cytotoxicity (CDC). Thus, in a preferred embodiment, the antibody and the Fc-containing fusion protein, respectively, of the present invention does not induce antibody-dependent cellular cytotoxicity (ADCC) and/or complement-dependent cytotoxicity (CDC).

In further preferred embodiments, the antibody and the Fc-containing fusion protein, respectively, of the present invention is capable of eliciting antibody dependent cellular phagocytosis (ADCP).

In further preferred embodiments, the antibody and the Fc-containing fusion protein, respectively, of the present invention is capable of mediating an Fc-based effector function by an antibody dependent cellular phagocytosis (ADCP).

Without being bound to theory, the capability of mediating an Fc-based effector function by an antibody dependent cellular phagocytosis (ADCP) can be tested as follows: Fluorescent beads can be coupled with antigen, and incubated with an antibody against the antigen. In the case of an ADCP-mediating antibody incubation of APCs with bead-Ag-antibody complexes leads to an enhanced uptake of fluorescent beads into APCs, as compared to beads-Ag without antibody and as compared to beads-Ag mixed with Fab2 fragments, demonstrating Fc-based ADCP. Uptake of fluorescent beads can be measured using flow cytometric assays.

In further preferred embodiments, the antibody and the Fc-containing fusion protein, respectively, of the present invention is capable of eliciting antibody dependent cellular phagocytosis (ADCP) via interaction with any of the above Fc receptors.

Without being bound to theory, the capability of eliciting antibody dependent cellular phagocytosis (ADCP) via interaction with any of the above Fc receptors can be tested as follows: To demonstrate ADCP via Fc receptors, APCs can be manipulated so that specific Fc receptors are reduced in expression, or completely knocked out using gene editing tools, such as CRISPR/Cas9. In addition, blocking specific Fc receptors with anti-Fc receptor antibodies can prevent binding of antibodies or Fc-Ag fusion proteins. Uptake of e.g. fluorescent beads coated with antigen after incubation with an antibody hence will be reduced in APCs in which the specific Fc receptor is reduced in expression by using e.g. silencing RNAs, microRNAs, short-hairpin RNAs, or CRISPR/Cas-mediated knock-out or in which the Fc receptor is blocked using an anti-Fc receptor antibody. Uptake of fluorescent beads can be measured by flow cytometry.

In further preferred embodiments, the antibody and the Fc-containing fusion protein, respectively, of the present invention is capable of eliciting antibody dependent cellular phagocytosis (ADCP) leading to the production of cytokines by the antigen presenting cell (APC).

Without being bound to theory, the capability of eliciting antibody dependent cellular phagocytosis (ADCP) leading to the production of cytokines by the antigen presenting cell (APC) can be tested as follows: APCs (e.g. monocyte-derived dendritic cells or macrophages) can be incubated with immune-complexes of antigen bound by antibody or by incubation with Fc-antigen fusion proteins, or by incubation with a mix of antibody-cross-linked Fc-Ag immune complexes (e.g. gB-Fc mixed with HDIT101). After one to two day incubation the supernatant is analysed for the presence of cytokines (e.g. IL1beta, IP10, TNFalpha, IL6, IFNgamma etc.) released by the APCs using commercially available assays, e.g. Luminex-based measurements or ELISAs. In case the antibody or Fc-Ag fusion protein mediates cytokine release through ADCP, this release is prevented in cells not expressing the specific Fc-receptor, i.e. cannot mediate ADCP or in cells in which the Fc receptor binding of the antibody or Fc-Ag fusion protein is inhibited with specific anti-Fc receptor antibodies.

In further preferred embodiments, the antibody and the Fc-containing fusion protein, respectively, of the present invention is capable of eliciting antibody dependent cellular phagocytosis (ADCP) leading to the generation and antigen presenting cell (APC) presentation of de novo HSV-derived peptides on MHC complexes.

Without being bound to theory, the capability of eliciting antibody dependent cellular phagocytosis (ADCP) leading to the generation and antigen presenting cell (APC) presentation of de novo HSV-derived peptides on MHC complexes can be tested as follows: APCs can be incubated with antibody bound to antigen, i.e. immune complexes or with Fc-Ag fusion proteins or a mix thereof. Cells are then lysed and MHC complexes can be immunoprecipitated using specific anti-MHC antibodies and the MHC immunopeptidome, i.e. the peptides bound and presented by the MHC can be identified using mass spectrometry. This approach will directly reveal the identity of the stimulated peptides. This approach can also be combined with stable isotope labeling to separate cellular peptides from antigen-derived peptides loaded on the MHC complex.

In further preferred embodiments, the antibody and the Fc-containing fusion protein, respectively, of the present invention is capable of eliciting antibody dependent cellular phagocytosis (ADCP) leading to the activation of a T-cell (and/or B-cell) immune response against de novo HSV-derived peptides.

Without being bound to theory, the capability of eliciting antibody dependent cellular phagocytosis (ADCP) leading to the activation of a T-cell (and/or B-cell) immune response against de novo HSV-derived peptides can be tested as follows: an approach to show T-cell stimulation by novel peptides presented by MHC complexes after ADCP via an antibody or an Fc-Ag fusion protein or a mix thereof as described in the present invention is to incubate APCs with antibody bound to Ag immune complexes, or with Fc-Ag fusion protein or a mix of Fc-Ag with antibody and then to inject these into an animal model of the malignant or infectious disease and to isolate the T cells after two weeks and to amplify the T-cell receptor nucleic acid sequences from total DNA or mRNA of circulating T-cells via polymerase chain reaction. Identification of the expansion of specific T cell clones in mice vaccinated with antibody-bound to antigen immune complexes or by Fc-Ag fusion proteins or a mix of Fc-Ag fusion protein with antibody which were absent before vaccination indicates for the presentation of de novo peptides via MHC. Similarly, B-cell receptor nucleic acid sequences can be amplified from circulating B cells and clonal expansion of B cells, i.e. clonal expansion of specific B-cell receptor sequences, that were absent before vaccination, indicate for a B cell response to de novo epitopes.

In further preferred embodiments, the antibody and the Fc-containing fusion protein, respectively, of the present invention the antibody is capable of eliciting antibody dependent cellular phagocytosis (ADCP) leading to the generation and antigen presenting cell (APC) presentation of HSV-derived peptides.

The vaccine for use in accordance with the above first and second aspect of the present invention is used for actively immunizing a subject.

Thus, the antibody and Fc-containing fusion protein, respectively, of the vaccine for use according to the first and second aspect of the present invention as defined above are particularly useful in medical settings.

Accordingly, in a preferred embodiment, the present invention relates to a vaccine pharmaceutical composition, comprising an effective amount of the antibody and Fc-containing fusion protein, respectively, as described above and at least one pharmaceutically acceptable excipient.

The term “vaccination”, “actively immunization” and the like are used herein to generally mean obtaining a desired pharmacological and/or physiological effect as described herein above in accordance with the rationale of the present invention.

The vaccine pharmaceutical composition of the present invention may be administered via a large range of classes of forms of administration known to the skilled person. Administration may be systemically, locally, orally, through aerosols including but not limited to tablets, needle injection, the use of inhalators, creams, foams, gels, lotions and ointments.

Preferably, the vaccine (or vaccine pharmaceutical composition) for use according to the first aspect of the present invention, is to be administered intravenously, topically, intradermally, subcutaneously, intra-cutanously, intramuscular an/or intrathecal.

Thus, preferably, the antibody and the Fc-containing fusion protein, respectively, as described in the context of the first aspect of the present invention as part of the vaccine (or vaccine pharmaceutical composition) is to be administered intravenously, topically, intradermally, subcutaneously, intra-cutanously, intramuscular an/or intrathecal.

Preferably, the vaccine (or vaccine pharmaceutical composition) for use according to the second aspect of the present invention, is to be administered intravenously, topically, intradermally, subcutaneously, intra-cutanously, intramuscular an/or intrathecal.

Thus, preferably, the antibody and the Fc-containing fusion protein, respectively, as described in the context of the second aspect of the present invention as part of the vaccine (or vaccine pharmaceutical composition) is to be administered intravenously, topically, intradermally, subcutaneously, intra-cutanously, intramuscular an/or intrathecal.

These routes of administration, i.e., via an intravenously, topically, intradermally, subcutaneously, intra-cutanously, intramuscular an/or intrathecal are known to the skilled person.

An excipient or carrier is an inactive substance formulated alongside the active ingredient, i.e., the antibody and the as described above of the present invention for the purpose of bulking-up formulations that contain potent active ingredients. Excipients are often referred to as “bulking agents,” “fillers,” or “diluents”. Bulking up allows convenient and accurate dispensation of a drug substance when producing a dosage form. They also can serve various therapeutic-enhancing purposes, such as facilitating drug absorption or solubility, or other pharmacokinetic considerations. Excipients can also be useful in the manufacturing process, to aid in the handling of the active substance concerned such as by facilitating powder flowability or non-stick properties, in addition to aiding in vitro stability such as prevention of denaturation over the expected shelf life. The selection of appropriate excipients also depends upon the route of administration and the dosage form, as well as the active ingredient and other factors.

Thus, the vaccine or vaccine pharmaceutical composition comprising an effective amount of the antibody and the Fc-containing fusion protein, respectively, of the present invention as described above may be in solid, liquid or gaseous form and may be, inter alia, in a form of (a) powder(s), (a) tablet(s), (a) solution(s) or (an) aerosol(s). It is preferred that said vaccine pharmaceutical composition optionally comprises a pharmaceutically acceptable carrier and/or diluent.

These vaccine or vaccine pharmaceutical compositions can be administered to the subject at a suitable dose. Administration of the suitable compositions may be effected by different ways as outlined above and may also be effected, e.g., by intravenous, intraperitoneal, subcutaneous, intramuscular, topical, intradermal, intranasal or intrabronchial administration.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Proteinaceous pharmaceutically active matter may be present in amounts between 1 ng and 10 mg/kg body weight per dose; however, doses below or above this exemplary range are envisioned, especially considering the aforementioned factors. If the regimen is a continuous infusion, it should also be in the range of 1 μg to 10 mg units per kilogram of body weight per minute.

Examples of suitable pharmaceutical carriers, excipients and/or diluents are well known in the art and include phosphate buffered saline solutions, water, emulsions, such as oil/water emulsions, various types of wetting agents, sterile solutions etc. Compositions comprising such carriers can be formulated by well known conventional methods. These vaccine pharmaceutical compositions can be administered to the subject at a suitable dose, i.e., in “an effective amount” which can easily be determined by the skilled person by methods known in the art. The dosage regimen will be determined by the attending physician and clinical factors. As is well known in the medical arts, dosages for any one patient depends upon many factors, including the patient's or subject's size, body surface area, age, the particular compound to be administered, sex, time and route of administration, general health, and other drugs being administered concurrently.

Thus, preferably, the antibody and the Fc-containing fusion protein, respectively, of the present invention as described above is included in an effective amount. The term “effective amount” refers to an amount sufficient to induce a detectable active immunization in the subject to which the vaccine pharmaceutical composition is to be administered. In accordance with the above, the content of the antibody and the Fc-containing fusion protein, respectively, of the present invention in the vaccine pharmaceutical composition is not limited as far as it is useful for actively immunizing as described above, but preferably contains 0.0000001-10% by weight per total composition. Further, the antibody described herein is preferably employed in a carrier. Generally, an appropriate amount of a pharmaceutically acceptable salt is used in the carrier to render the composition isotonic. Examples of the carrier include but are not limited to saline, Ringer's solution and dextrose solution. Preferably, acceptable excipients, carriers, or stabilisers are non-toxic at the dosages and concentrations employed, including buffers such as citrate, phosphate, and other organic acids; salt-forming counter-ions, e.g. sodium and potassium; low molecular weight (>10 amino acid residues) polypeptides; proteins, e.g. serum albumin, or gelatine; hydrophilic polymers, e.g. polyvinylpyrrolidone; amino acids such as histidine, glutamine, lysine, asparagine, arginine, or glycine; carbohydrates including glucose, mannose, or dextrins; monosaccharides; disaccharides; other sugars, e.g. sucrose, mannitol, trehalose or sorbitol; chelating agents, e.g. EDTA; non-ionic surfactants, e.g. Tween, Pluronics or polyethylene glycol; antioxidants including methionine, ascorbic acid and tocopherol; and/or preservatives, e.g. octadecyldimethylbenzyl ammonium chloride; hexamethonium chloride; benzalkonium chloride, benzethonium chloride; phenol, butyl or benzyl alcohol; alkyl parabens, e.g. methyl or propyl paraben; catechol; resorcinol; cyclohexanol; 3-pentanol; and m-cresol). Suitable carriers and their formulations are described in greater detail in Remington's Pharmaceutical Sciences, 17th ed., 1985, Mack Publishing Co.

Active immunization can be monitored by periodic assessment by the skilled person by applying routine methods as outlined above.

The vaccine's antibody and the Fc-containing fusion protein, respectively, of the present invention or the vaccine pharmaceutical composition of the invention may be in sterile aqueous or non-aqueous solutions, suspensions, and emulsions as well as creams and suppositories. Examples of non-aqueous solvents are propylene glycol, polyethylene glycol, vegetable oils such as olive oil, and organic esters such as ethyl oleate. Aqueous carriers include water, alcoholic/aqueous solutions, emulsions or suspensions, including saline and buffered media. Preservatives and other additives may also be present such as, for example, antimicrobials, anti-oxidants, chelating agents, and inert gases and the like. Furthermore, the pharmaceutical composition of the invention may comprise further agents depending on the intended use of the pharmaceutical composition. Said agents may be, e.g., polyoxyethylene sorbitan monolaurate, available on the market with the commercial name Tween, propylene glycol, EDTA, Citrate, Sucrose as well as other agents being suitable for the intended use of the pharmaceutical composition that are well-known to the person skilled in the art.

In accordance with this invention, the terms “vaccine” and “vaccine pharmaceutical composition” relates to a vaccine composition for administration to a patient, preferably a human patient.

As shown in the appended examples, the vaccine according to the present invention surprisingly leads to markedly longer time-to-first-recurrence and a lower recurrence rate in patients having a severe infectious disease (see Example 8).

Moreover, a pharmacokinetic time-to-event (TTE) modeling analysis revealed that a multiple administration of the vaccine surprisingly leads to higher median probabilities of being recurrence/lesion-free for a longer time (see Example 9).

This leads to the provision of further preferred embodiments of the present invention wherein, in a nutshell,
(i) a specific subgroup of patients can beneficially be vaccinated; and/or
(ii) a specific beneficial prime-boost dosage regimen is provided.

This is in line with the above-formulated technical problem underlying the present invention (according to which further means and methods for the treatment and/or prevention of (recurrent) infectious and malignant diseases, in particular, with respect to long-term or persistent effects are provided wherein, inter alia, the individuals/subjects that have to be treated simultaneously acquire a long term immunity against said infectious or malignant diseases in terms of a vaccination).

These further embodiments of the present invention are individually described in the following for the first and second aspect of the present invention.

First, in the following, a specific subgroup of patients and/or a specific prime-boost dosage regimen in relation to the first aspect of the present invention (relating to a vaccine for use in actively immunising a subject against an infectious disease or a malignant disease, said vaccine comprising: (a) an antibody against an antigen correlated with said infectious disease or malignant disease; or (b) an Fc-containing fusion protein comprising an Fc part of an antibody fused to an antigen correlated with said infectious disease or malignant disease, wherein said Fc part is capable of binding a receptor present on or in an antigen presenting cell (APC) selected from the group consisting of: Type I: activatory FcγRI, FcγRIIa, FcγRIIc, FcγRIIIa, FcγRIIIb, and inhibitory FcγRIIb; and Type II: neonatal FcR (FcRn) and cytosolic TRIM21), is described in more detail:

Accordingly, in a preferred embodiment, the present invention relates to a vaccine comprising: (a) an antibody against an antigen correlated with said infectious disease or malignant disease, wherein said vaccine is to be administered to a subject having an acute recurrence of said infectious disease or of said malignant disease and suffering from a chronic (and/or severe chronic) (infectious) disease or from a chronic (and/or severe chronic) (malignant) disease with more than 4 recurrences/year.

As outlined above, in the context of the vaccine for use in actively immunizing a subject against an infectious disease or malignant disease, it requires, in a preferred embodiment, the presence of an acute recurrence (in terms of an acute/persistent infection) or an acute recurrence of a malignant disease (in terms of an acute/persistent malignant disease), and, accordingly, it requires the presence of antigen. This, in particular, relates to the aspect (a) wherein an antibody against an antigen correlated with said infectious disease or malignant disease is used as a component of the vaccine of the present invention. This is because, for being able of actively immunizing the subject in line with the rationale of the present invention, not only the antibody but also an antigen correlated with said infectious disease or malignant disease must be present.

Moreover, in accordance with this preferred embodiment, the subject preferably suffers from a chronic (preferably a severe chronic) infectious disease or a chronic (preferably a severe chronic) malignant disease with more than 4 recurrences/year.

A “severe infectious disease”/“chronic infectious disease”/“severe chronic infectious disease” or a “severe malignant disease”/“chronic malignant disease”/“severe chronic malignant disease” is a disease, which presents with/shows several recurrences/year.

The term “chronic” is generally understood in medical settings as a (health) condition or disease (also known as chronic disease or chronic illness) that is persistent or otherwise long-lasting in its effects or a disease that comes with time. The term “chronic” is often applied when the course of the disease lasts for more than three months.

The term “recurrence” is commonly understood in the art in the context of medical settings as the return or re-occurrence of a previous condition characteristic for said disease and may, e.g., be a fresh or repeated outbreak of the disease and/or of a symptom. The skilled person is easily in a position to determine the start/appearance of said outbreak or symptom, i.e., the start or onset of the recurrence in terms of the present invention.

The severity of the chronic disease depends on the number of recurrences/year.

In preferred embodiments, the subject to be treated has more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 recurrences/year.

In a more preferred embodiment, the subject to be treated has more than 4 recurrences/year.

In another preferred embodiment, when the severe infectious disease is an HSV-associated disease, preferably a severe HSV infection, the recurrence is the recurrence of lesions.

As mentioned, the context of the first aspect of the present invention, the present invention relates to a specific dosage regimen.

Accordingly, in another preferred embodiment, the present invention relates to a vaccine comprising: (a) an antibody against an antigen correlated with said infectious disease or malignant disease, wherein said vaccine is to be administered by a prime-boost dosage regimen by the following administration steps:
(i) administering a first dosage of the vaccine to the subject during the first week of recurrence, preferably, immediately upon start of the first recurrence (prime administration);
(ii) administering a further dosage of the vaccine to the subject during the first week of the second recurrence, preferably, immediately upon start of the second recurrence (boost administration); and
(iii) optionally repeating step (ii) during the first week of every further recurrence, preferably, immediately upon start of every further recurrence (further boost administrations),

wherein one prime and at least one boost administration/year are to be administered.

In a further preferred embodiment, in the context of the first aspect of the present invention, the above and below described specific dosage regimen preferably is to be administered to a subject suffering from a chronic (and/or severe chronic) (infectious) disease or from a chronic (and/or severe chronic) (malignant) disease with more than 4 recurrences/year.

In the context of the present invention, a “week” refers to a period of time of 7 days.

In the context of the present invention, a “month” refers to a period of time of 4 weeks.

The prime administration and the boost administration, respectively, are to be administered during the first week of recurrence, preferably, immediately upon start of the recurrence of the disease.

As explained above, the recurrence, return, re-occurrence or repeated outbreak of the respective disease is easily determined by the skilled person and, preferably, is a time point wherein disease specific symptoms can be detected.

In preferred embodiment, when the infectious disease is a HSV-associated disease, preferably a severe HSV infection, the recurrence is the recurrence of lesions.

The time point of the prime administration and the boost administration, respectively, does not necessarily have to be exactly during the first week of recurrence and not necessarily exactly immediately upon the recurrence of the disease, respectively. An adequate time point can be determined within a suitable range according to conditions of a subject or a patient. Accordingly, in preferred embodiments, a specific prime administration can be 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days after the start of the recurrence of the disease.

In more preferred embodiments, a specific prime administration is at day 0 (i.e., on the same day of the recurrence of the disease).

In more preferred embodiments, a specific prime administration is during the first week, even more preferably at day 1, day 2, day 3, day 4, day 5, day 6 or day 7 after the start of the recurrence of the disease.

The same preferred embodiments apply, mutatis mutandis, to the one or more boost administration(s).

As outlined, the boost administration is repeated for every further recurrence (further boost administrations).

Accordingly, in a preferred embodiment, a prime administration of the vaccine of the present invention is to be administered upon the start or after the first recurrence of the disease within the above limitations, while an n^{th} boost administration (n is an integer of 1 or greater) is to be administered upon start of or after the n^{th} recurrence of the disease (n is an integer of 1 or greater) within the above limitations.

As outlined, in the prime and boost regimen, in a preferred embodiment, one prime and at least one boost administration/year are to be administered.

In further preferred embodiments, one prime and at least two boost administrations/year are to be administered.

In further preferred embodiments, one prime and at least three boost administrations/year are to be administered.

In further preferred embodiments, one prime and at least four, five, six, seven, eight, nine, ten, eleven, twelve, or more boost administrations/year are to be administered.

In further preferred embodiments, one prime and n^{th} boost administrations/year (n is an integer of 1 or greater) are to be administered.

In another preferred embodiment, the present invention relates to a vaccine comprising: (b) an Fc-containing fusion protein comprising an Fc part of an antibody fused to an antigen correlated with said infectious disease or malignant disease, wherein said Fc part is capable of binding a receptor present on or in an antigen presenting cell (APC) selected from the group consisting of: Type I: activatory FcγRI, FcγRIIa, FcγRIIc, FcγRIIIa, FcγRIIIb, and inhibitory FcγRIIb; and Type II: neonatal FcR (FcRn) and cytosolic TRIM21, wherein said vaccine is to be administered to a subject suffering from a chronic (and/or severe chronic) (infectious) disease or from a chronic (and/or severe chronic) (malignant) disease with more than 4 recurrences/year.

As regards preferred embodiments, the same applies, mutatis mutandis, as has been set forth above in the context of a specific subgroup of patients in relation to the vaccine comprising: (a) an antibody of the present invention as defined above.

In another preferred embodiment, when the severe infectious disease is a HSV-associated disease, preferably a (severe) chronic HSV infection, the recurrence is the recurrence of lesions.

Moreover, as regards a specific dosage regimen (i.e., a prime-boost administration) of the vaccine comprising: (b) an Fc-containing fusion protein comprising an Fc part of an antibody fused to an antigen correlated with said infectious disease or malignant disease, wherein said Fc part is capable of binding a receptor present on or in an antigen presenting cell (APC) selected from the group consisting of: Type I: activatory FcγRI, FcγRIIa, FcγRIIc, FcγRIIIa, FcγRIIIb, and inhibitory FcγRIIb; and Type II: neonatal FcR (FcRn) and cytosolic TRIM21, in another preferred embodiment, the present invention relates to said vaccine, wherein said vaccine is to be administered by a prime-boost dosage regimen by the following administration steps:
(i) administering a first dosage of the vaccine to the subject (prime administration);
(ii) administering a further dosage of the vaccine to the subject in the interval between 15 to 30 days after the prime administration (boost administration); and
(iii) optionally repeating step (ii) in the interval between 15 to 30 days after the previous boost administration (further boost administrations).

“Interval” (an interval between individual administrations) indicates an interval between administration of the n^{th} dose (n is an integer of 1 or greater) and administration of the (n+1)^{th} dose.

An adequate interval between the first (prime) dosage and the further (boost) dosage and between the further (boost) dosage and the correspondingly subsequent further (boost) dosage, respectively, can be determined within a suitable range according to conditions of a subject or a patient. A specific administration interval between the first (prime) dosage and the further (boost) dosage and between the further (boost) dosage and the correspondingly subsequent further (boost) dosage, respectively, is 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days or 45 days.

In preferred embodiments, the interval between the first dosage and the further (boost) dosage and between the further (boost) dosage and the correspondingly further (boost) dosage, respectively, phase is 5 days to 45 days. In further preferred embodiments, the interval is 5 days to 45 days. In further preferred embodiments, the interval is 5 days to 40 days. In further preferred embodiments, the interval is 5 days to 35 days. In further preferred embodiments, the interval is 5 days to 30 days. In further preferred embodiments, the interval is 5 days to 25 days. In further preferred embodiments, the interval is 5 days to 20 days. In further preferred embodiments, the interval is 5 days to 15 days. In further preferred embodiments, the interval is 5 days to 10 days.

In further preferred embodiments, the interval is 10 days to 45 days. In further preferred embodiments, the interval is 10 days to 40 days. In further preferred embodiments, the interval is 10 days to 35 days. In further preferred embodiments, the interval is 10 days to 30 days. In further preferred embodiments, the interval is 10 days to 25 days. In further preferred embodiments, the interval is 10 days to 20 days. In further preferred embodiments, the interval is 10 days to 15 days.

In further preferred embodiments, the interval is 15 days to 45 days. In further preferred embodiments, the interval is 15 days to 40 days. In further preferred embodiments, the interval is 15 days to 35 days. In further preferred embodiments, the interval is 15 days to 30 days. In further preferred embodiments, the interval is 15 days to 25 days. In further preferred embodiments, the interval is 15 days to 20 days.

In further preferred embodiments, the interval is 20 days to 45 days. In further preferred embodiments, the interval is 20 days to 40 days. In further preferred embodiments, the interval is 20 days to 35 days. In further preferred embodiments, the interval is 20 days to 30 days. In further preferred embodiments, the interval is 20 days to 25 days.

In further preferred embodiments, the interval is 25 days to 45 days. In further preferred embodiments, the interval is 25 days to 40 days. In further preferred embodiments, the interval is 25 days to 35 days. In further preferred embodiments, the interval is 25 days to 30 days.

In further preferred embodiments, the interval is 30 days to 45 days. In further preferred embodiments, the interval is 30 days to 40 days. In further preferred embodiments, the interval is 30 days to 35 days.

In the most preferred embodiments, the interval is 15 or 30 days.

In other preferred embodiments, the further (boost) dosage and the correspondingly subsequent further (boost) dosage, respectively, is administered 1 day, 2 days, 3 days, 4, days, 5 days, 6 days, 7 days (1 week), 8 days, 9 days, 10 days, 11 days, 12 days, 13 days, 14 days (2 weeks), 15 days, 16 days, 17 days, 18 days, 19 days, 20 days, 21 days (3 weeks), 22 days, 24 days, 25 days, 26 days, 27 days, 28 days (4 weeks), 29 days, 30 days or more days after the the first (prime) dosage and the further (boost) dosage, respectively.

In more preferred embodiments, the interval in the further (boost) dosage and the correspondingly subsequent further (boost) dosage, respectively, is administered 15 days, 20 days, 25 days, or 30 days after the the first (prime) dosage and the further (boost) dosage, respectively.

Second, in the following, a specific subgroup of patients and/or a specific prime-boost dosage regimen in relation to the second aspect of the present invention (relating to a vaccine for use in actively immunizing a subject against an infectious disease, wherein said infectious disease is an HSV-associated disease; and (a) wherein said antibody is an anti-HSV antibody and said subject suffers from an acute HSV-associated disease, preferably an acute HSV infection; or (b) wherein in said Fc-containing fusion protein comprising an Fc part of an antibody fused to an antigen, the antigen correlates with an HSV-associated disease), is described in more detail:

Accordingly, in a preferred embodiment, the present invention relates to a vaccine for use in actively immunizing a subject against an infectious disease, wherein said infectious disease is an HSV-associated disease; and (a) wherein said antibody is an anti-HSV antibody and said subject suffers from an acute HSV-associated disease, preferably an acute HSV infection, wherein said vaccine is to be administered to a subject having an acute recurrence of said disease and suffering from a chronic (and/or severe chronic) HSV-associated disease, preferably, a chronic (and/or severe chronic) HSV infection, with more than 4 recurrences/year.

As regards preferred embodiments of this specifically defined group of patients/subjects (i.e., the subject suffering from a chronic (and/or severe chronic) HSV-associated disease, preferably, a chronic (and/or severe chronic) HSV infection, with more than 4 recurrences/year), the same applies, mutatis mutandis, as has been set forth above in the context of the specific subgroup of patients in relation to the first aspect of the present invention.

As mentioned, the context of the second aspect of the present invention, the present invention relates to a specific dosage regimen.

Accordingly, in another preferred embodiment, the present invention relates to a vaccine for use in actively immunizing a subject against an infectious disease, wherein said infectious disease is an HSV-associated disease; and (a) wherein said antibody is an anti-HSV antibody and said subject has an acute recurrence of said disease and suffers from a chronic (and/or severe chronic) HSV-associated disease, preferably, a chronic (and/or severe chronic) HSV infection, wherein said vaccine is to be administered by a prime-boost dosage regimen by the following administration steps:
(i) administering a first dosage of the vaccine to the subject during the first week of recurrence, preferably, immediately upon start of the first recurrence (prime administration);
(ii) administering a further dosage of the vaccine to the subject during the first week of recurrence, preferably, immediately upon start of the second recurrence (boost administration); and
(iii) optionally repeating step (ii) during the first week of every further recurrence, preferably, immediately upon start of every further recurrence (further boost administrations),

wherein one prime and at least one boost administration/year are to be administered.

As regards preferred embodiments of this specifically defined dosage regimen, the same applies, mutatis mutandis, as has been set forth above in the context of the specific dosage regimen in relation to the first aspect of the present invention.

In another preferred embodiment, the present invention relates to a vaccine for use in actively immunizing a subject against an infectious disease, wherein said infectious disease is an HSV-associated disease; and wherein the vaccine comprises: (b) an Fc-containing fusion protein comprising an Fc part of an antibody fused to an antigen correlated with said infectious disease or malignant disease, wherein said Fc part is capable of binding a receptor present on or in an antigen presenting cell (APC) selected from the group consisting of: Type I: activatory FcγRI, FcγRIIa, FcγRIIc, FcγRIIIa, FcγRIIIb, and inhibitory FcγRIIb; and Type II: neonatal FcR (FcRn) and cytosolic TRIM21, wherein in said Fc-containing fusion protein comprising an Fc part of an antibody fused to an antigen, the antigen correlates with an HSV-associated disease and wherein said vaccine is to be administered to a subject suffering from a (severe) chronic HSV-associated disease, preferably, a (severe) chronic HSV infection, with more than 4 recurrences/year.

As regards preferred embodiments of this specifically defined group of patients/subjects (i.e., the subject suffering from a (severe) chronic HSV-associated disease, preferably, a (severe) chronic HSV infection, with more than 4 recurrences/year), the same applies, mutatis mutandis, as has been set forth above in the context of the specific subgroup of patients in relation to the first aspect of the present invention.

Moreover, as regards a specific dosage regimen (i.e., a prime-boost administration) of the vaccine comprising: (b) an Fc-containing fusion protein comprising an Fc part of an antibody fused to an antigen correlated with said infectious disease or malignant disease, wherein said Fc part is capable of binding a receptor present on or in an antigen presenting cell (APC) selected from the group consisting of: Type I: activatory FcγRI, FcγRIIa, FcγRIIc, FcγRIIIa, FcγRIIIb, and inhibitory FcγRIIb; and Type II: neonatal FcR (FcRn) and cytosolic TRIM21, wherein in said Fc-containing fusion protein comprising an Fc part of an antibody fused to an antigen, the antigen correlates with an HSV-associated disease and wherein said vaccine is to be administered by a prime-boost dosage regimen by the following administration steps:
(i) administering a first dosage of the vaccine to the subject (prime administration);
(ii) administering a further dosage of the vaccine to the subject in the interval between 15 to 30 days after the prime administration (boost administration); and
(iii) optionally repeating step (ii) in the interval between 15 to 30 days after the previous boost administration (further boost administrations).

As regards preferred embodiments of this specifically defined dosage regimen, the same applies, mutatis mutandis, as has been set forth above in the context of the specific dosage regimen in relation to the first aspect of the present invention.

The invention also relates to method of actively immunizing a subject against an infectious disease or malignant disease in a subject as defined herein above in the context of the first aspect of the present invention.

The invention also relates to method of actively immunizing a subject against an HSV-associated disease in a subject as defined herein above in the context of the second aspect of the present invention.

As regards the preferred embodiments of the method for actively immunizing the same applies, mutatis mutandis, as has been set forth above in the context of the antibody and the Fc-containing fusion protein, respectively, the vaccine or the vaccine pharmaceutical composition for use as defined above.

In the present invention, the subject is, in a preferred embodiment, a mammal such as a dog, cat, pig, cow, sheep, horse, rodent, e.g., rat, mouse, and guinea pig, or a primate, e.g., gorilla, chimpanzee, and human. In a most preferable embodiment, the subject is a human.

In further preferred embodiments, in particular regarding the second aspect of the present invention, said disease is selected from the group consisting of Herpes simplex labialis, Herpes simplex genitalis, chronic or disseminated cutaneous herpes simplex infection, Herpes gladiatorum, Eczema herpeticum, Herpes keratoconjunctivitis, Herpes neonatorum, Alzheimer disease (AD), HSV pneumonia, Bell's palsy, Herpes esophagitis, Herpesviral encephalitis and Herpesviral meningitis, Herpetic sycosis, Herpes withlow, Herpes gingivostomatitis, presence of an oral recidive, presence of a genital recidive, eczema herpeticatum, herpes neonatorum, immune deficiency, immunocompromised patients, resistance against a virusstatic agent, encephalitis, meningitis, meningoencephalitis, eye infections, and/or generalized HSV infections.

Other aspects and advantages of the invention will be described in the following examples, which are given for purposes of illustration and not by way of limitation. Each publication, patent, patent application or other document cited in this application is hereby incorporated by reference in its entirety.

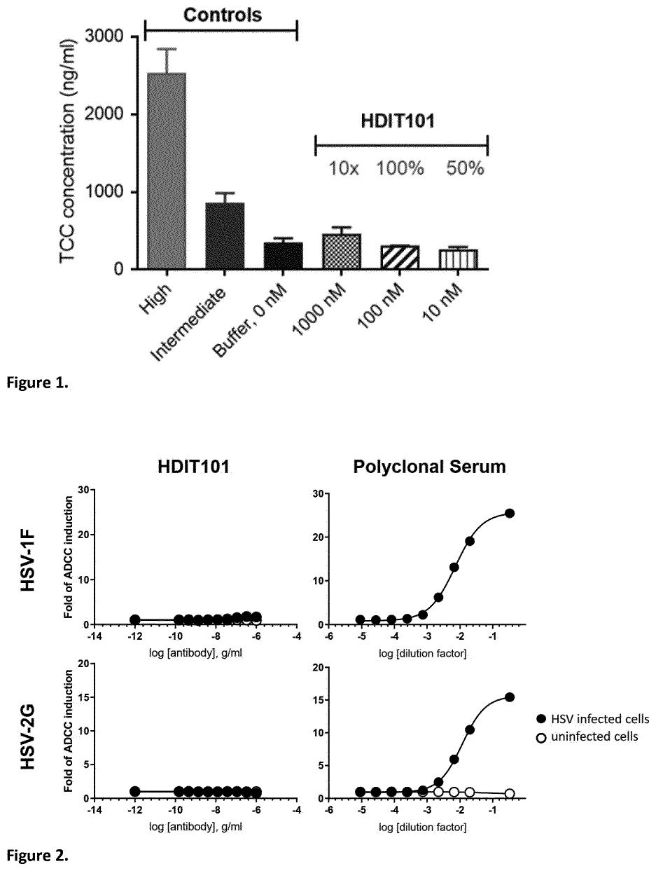

FIG. 1: HDIT101 does not induce complement-dependent cytotoxicity (CDC)

Complement-dependent cytotoxicity (CDC) was measured by determining the formation of Cb5-9 terminal complement complex (TCC). 150 plaque forming units of HSV-2G were incubated with HDIT101 at different concentrations and complement source (HSV-seronegative serum vs. heat-inactivated HSV-seronegative serum). HDIT101 concentrations were 100 nM (neutralizing concentration), 1000 nM (10× neutralizing concentration, excess of antibodies) or 10 nM (50% neutralizing concentration). Positive controls (high and intermediate C5b-9 concentration controls) and negative control (buffer only) were included. C5b was measured using an enzyme-linked immunosorbent assay. mAb mouse anti-human C5b-9 (clone aE11) was coated on plates. Sample or controls containing complement-complex were added. Goat anti human C5 antibody was added, followed by incubation with F(ab)2, rabbit anti goat IgG (H+L) peroxidase coupled. Substrate was added and colour change measured using a photometer. Zymosan-activated normal human serum, calibrated against purified C5b-9, was used as standard. At no tested HDIT101 concentration substantial induction of C5b-9 was observed as compared to intermediate and high control.

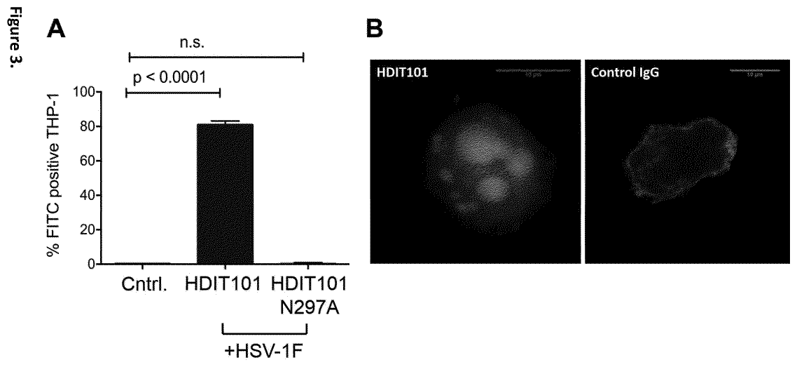

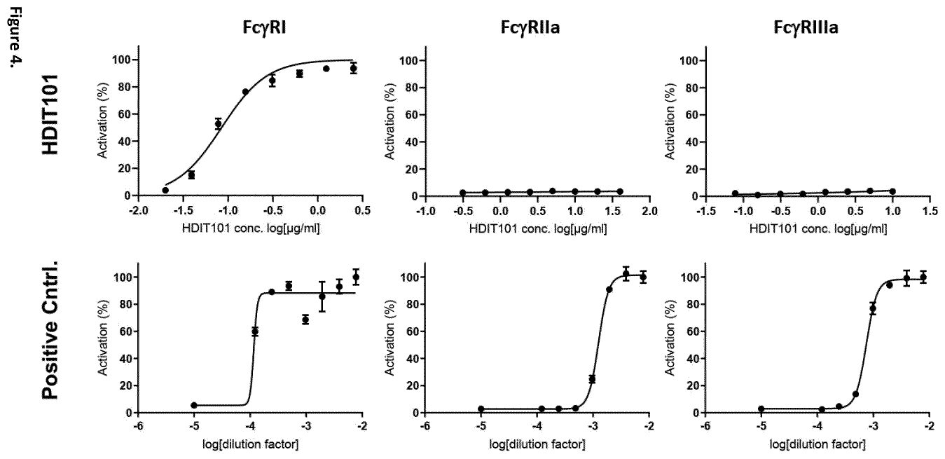

FIG. 2: HDIT101 does not induce antibody-dependent cytotoxicity (ADCC)

Measurement of ADCC was performed using a commercially available reporter assay (ADCC Reporter Bioassay, Core Kit, Promega #G7018). Target cells (HSV-1 or HSV-2-infected Vero cells) were incubated with HDIT101 or polyclonal serum as control in a series of dilutions and effector cells (engineered Jurkat cells stably expressing CD16 (FcγRIIIa) and an NFAT-Luciferase reporter gene). Once ADCC is induced, the NFAT-luciferase reporter gene is activated, and luciferase luminescence can be determined by adding substrate and measuring relative light units using a luminometer. Fold change to samples without antibody or serum were calculated and plotted. At no tested HDIT101 concentration, substantial induction of FcgRIIIa-induced NFAT signaling was observed in reporter cells, as opposed to polyclonal serum.

FIG. 3: HDIT101 induces antibody-dependent phagocytosis (ADCP) in THP-1 cell line THP-1 cells were incubated with antibody-HSV-1F immune complexes for 18 h. The cells were fixed using paraformaldehyde, permeabilized and stained with an anti-human IgG coupled to FITC. Stained cells were analysed using a flow cytometer (A) or fluorescence microscope (B). Significant increase of fluorescent cells over a control antibody, deglycosylated HDIT101 N297A, or unstained control were observed.

FIG. 4: HDIT101 opsonized HSV infected cells bind to FcgRI, but not to FcgRIIa or FcgRIIa expressing cells

Murine Thymoma-cells (BW5147) were modified to stably express a chimeric protein consisting of the extracellular domains of the human FcgRs I, IIa, IIb, or IIIa coupled to the TCR-zeta domain. Activation of the receptors by binding of IgGs leads to IL-2 secretion in this model, which then can be measured via ELISA in the supernatant. HSV-1 infected Vero cells were incubated with serial dilutions of HDIT101 or a positive control (Cytotect) and IL-2 secretion was measured. HDIT101 opsonized infected cells activated IL-2 secretion from cells expressing FcgRI ectodomain fusion protein, but not for cells expressing FcgRIIa or FcgRIIIa fusion proteins.

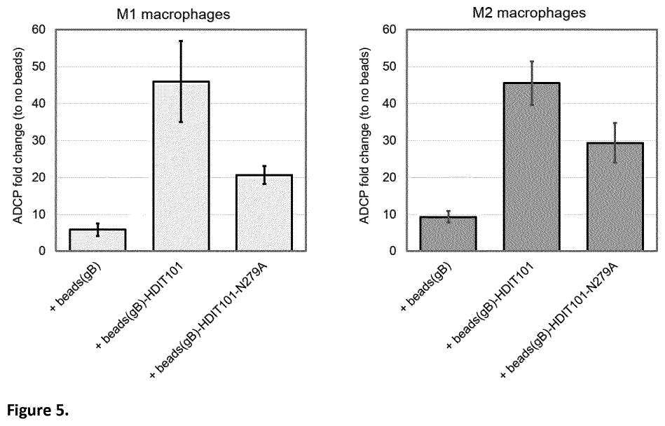

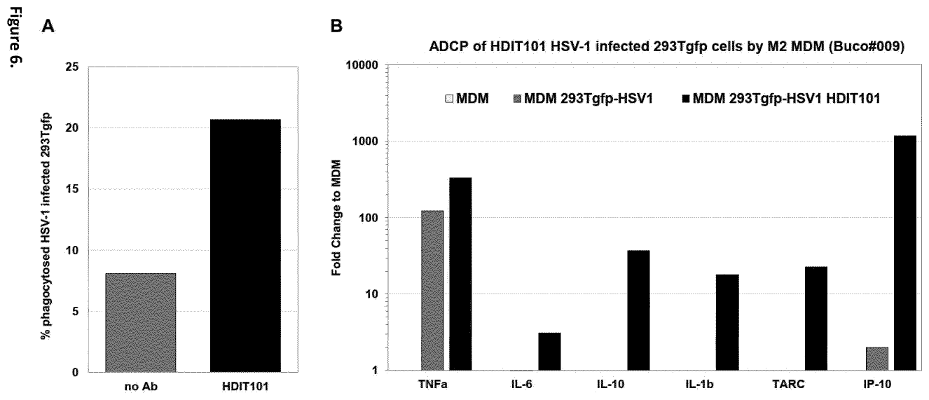

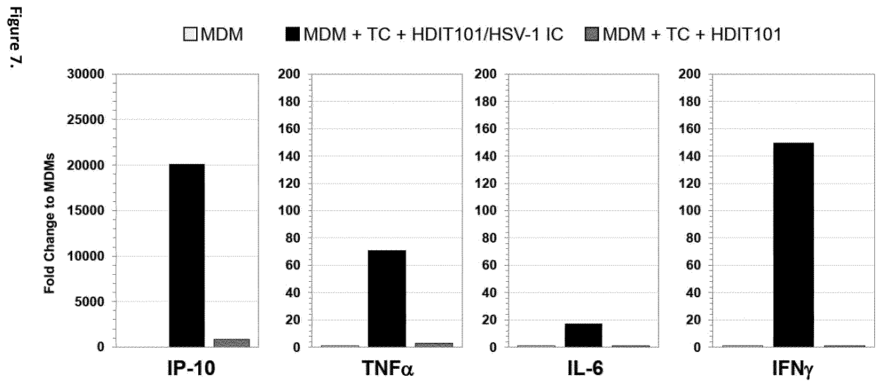

FIG. 5: HDIT101 induces ADCP in monocyte-derived macrophages (MDM) of M1 and M2 type

Fluorescent streptavidin coated beads were decorated with biotinylated gB and then complexed with HDIT101 or the mutant HDIT101-N279A. Monocyte derived M1 or M2 macrophages were incubated for 2 h with the respective bead complex and subsequently analysed via flow cytometry. The mean fluorescence intensity (MFI) is displayed as fold change to the control condition with untreated macrophages. The data indicates mean values ±SD of three different donors. Phagocytosis was increased by HDIT101 to approximately 50-fold, while HDIT101-N297A induced phagocytosis was significantly reduced.

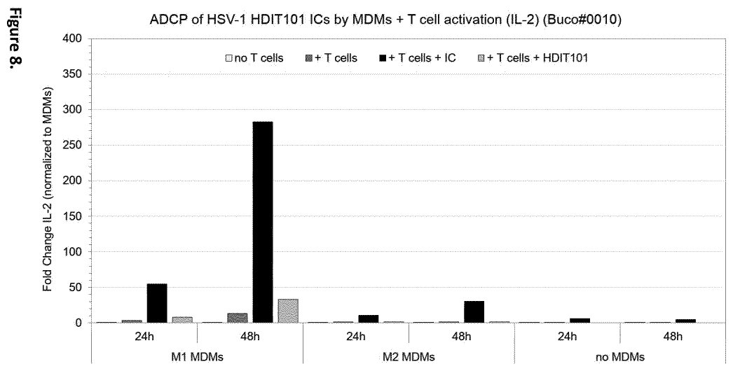

FIG. 6: HDIT101 opsonized HSV-1F infected cells induce IP10 cytokine production in MDM

293Tgfp cells were infected with HSV-1F and opsonized with a saturating concentration of HDIT101. Monocyte-derived macrophages (MDM) of type M2 were differentiated from CD14-positive cells from a blood donor (#Buco009) using macrophage colony-stimulating factor (M-CSF). MDMs were incubated with HSV-1 infected and HDIT101-opsonized 293Tgfp cells for 2 h and ADCP was measured by counting GFP-positive CD11-positive cells using fluorescence microscopy imaging (A). A parallel sample was incubated for 24 h and the supernatant was harvested and subjected to cytokine measurement using LEGENDplex™ Human M1/M2 Macrophage Panel (10-plex) from Biolegend (B). Incubation of MDMs with HDIT101-opsonized, but not with non-opsonized HSV-1 infected 293Tgfp cells, strongly induced cytokines, in particular IP-10 (CXCL10) by >1000-fold.

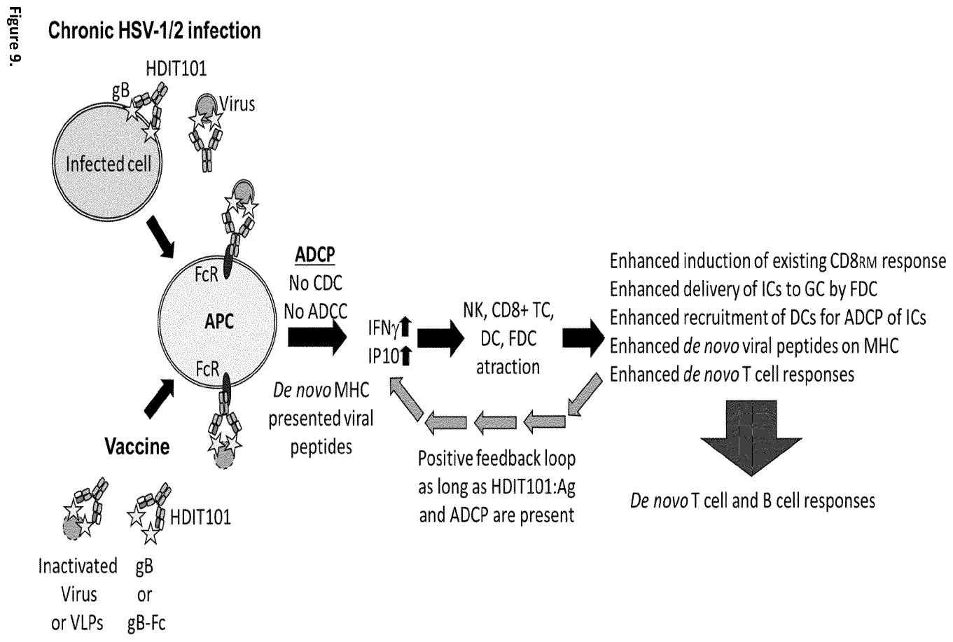

FIG. 7: HDIT101 opsonized viral particles induce augmented IP10 cytokine response in presence of MDMs and autologous T cells

Monocyte-derived macrophages (MDM) of type M2 were differentiated from CD14-positive cells from a blood donor (#Buco009) using macrophage colony-stimulating factor (M-CSF). HSV-1F immune complexes (ICs) with HDIT101 were generated by 1 h incubation of the virus in a HDIT101 concentration that neutralizes viral infectivity to 100% in Vero cells. ICs or HDIT101 alone were then transferred to MDMs and mixed with autologous T cells (TC). T cells were preactivated at the time of monocyte isolation for 2 days using anti-CD28/CD3 beads in the presence of IL7/15. Activation was stopped two days later by washing out the beads and T cells were rested for 7 days in IL7/15 containing media before addition to the MDMs. Addition of HSV-1F ICs with HDIT101 strongly induced IFNg by ^{˜}150-fold and most strongly IP-10 by 20,000-fold.

FIG. 8: HSV-1: HDIT101 ICs stimulated autologous T-cell IL-2 production predominantly after ADCP by M1 type MDMs and moderately by M2 type MDMs

CD14-positive monocytes were isolated from HSV-seropositive blood donor (Buco #0010) and differentiated either using M-CSF to type M2 MDMs, or granulocyte-MCSF (GM-CSF) to type M1 MDMs. HSV-1F immune complexes (ICs) with HDIT101 were generated by 1 h incubation of the virus in a HDIT101 concentration that neutralizes virus infectivity to 100% in Vero cells. Autologous T cells were generated and resting unstimulated T cells were added to the macrophages or left alone (no MDMs). HDIT101:HSV-1 ICs or HDIT101 antibody were added to MDM T cell co-cultures, and supernatants were analysed using Human Th1 LEGENDplex™ (5-plex) kit from Biolegend for IL-2 concentration 24 h or 48 h later. In the presence of HDIT101:HSV-1 ICs, but not in the presence of HDIT101 alone, IL-2 levels increased substantially. Of note, M1 MDMs supported much stronger IL-2 induction (>250-fold) as compared to M2 MDMs (^{˜}40-fold) after 48 h of stimulation with HDIT101:HSV-1 ICs. Presence of MDMs and ICs was required for the induction of IL-2 production by the T cells.

FIG. 9: Proposed mechanism of induction of de novo B-/T-cell responses through HDIT101-induced ADCP

HSV-1-infected cells or HSV-1 particles bind to HDIT101 and HDIT101 induces ADCP through binding to FcgRI. Of note, HDIT101 does not induce ADCC, or CDC. ADCP of HDIT101-ICs leads to presentation of viral peptides on MHC I or MHC II complexes and of note to the induction of IP10, which serves as a chemotactic to attract dendritic cells, NK and T cells to the site of infection. Follicular dendritic cells (FDCs) take up intact HDIT101-opsonized ICs and transport them to germinal centres (GC) where de novo B cell responses are induced to epitopes revealed through Fab binding-induced structural changes in gB within the IC. Existing tissue resident memory T cell responses (CD8_{RM}) are enhanced and migrate towards site of HSV replication and de novo T cell responses are induced when circulating naïve T cells recognize viral T cell epitopes presented on the MHC complexes. The unique feature of HDIT101 to mediate ADCP and induce IP10 and IFNg supports the generation of de novo T-cell (and B-cell) responses. These features can be used for vaccine design, whereby inactivated virus or virus like particles or recombinant gB or gB-Fc protein are complexed with HDIT101 and serve as a vaccine.

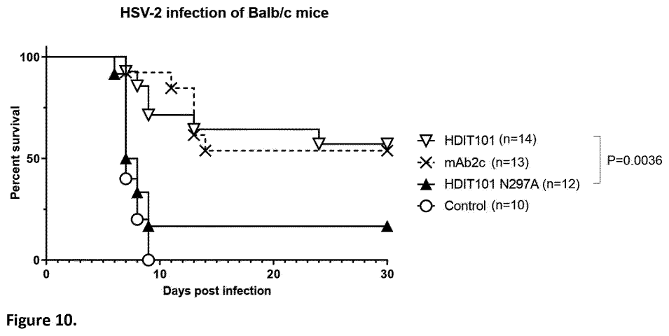

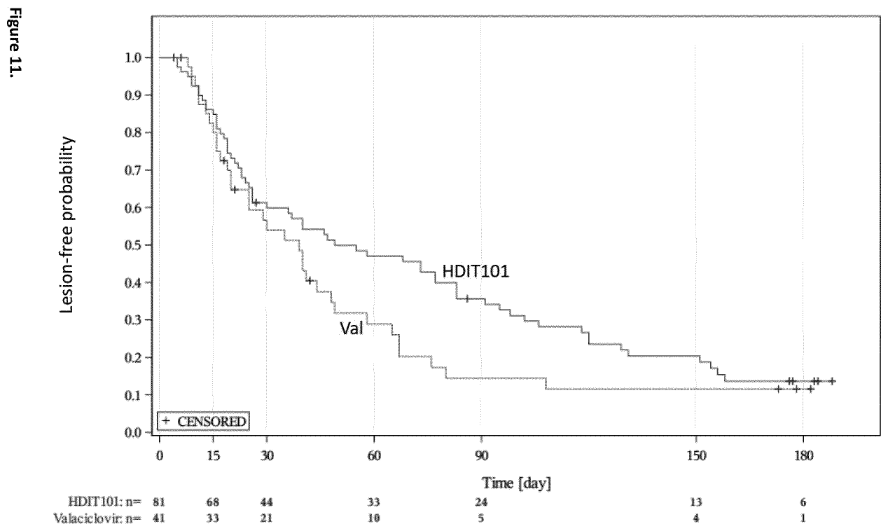

FIG. 10: HDIT101 N297A is deficient to rescue immunocompetent Balb/c mice from a lethal HSV-2G infection

Female immunocompetent Balb/c OlaHsd mice at an age of 8 weeks were infected intravaginally with 5×10e5 plaque forming units HSV-2G and treated 14 h later intravenously with 300 g HDIT101 (n=14), HDIT101 N297A (n=12), murine mAb2c (n=13) or PBS (n=10). Survival was monitored for 30 days post infection. Treatment with HDIT101 or murine mAb2c increased survival within 30 days to 50%, while all control mice died within 10 days. HDIT101 N297A treated mice showed significantly reduced survival as compared to HDIT101 treated mice (p=0.0036, Gehan-Breslow-Wilcoxon statistical test).

FIG. 11: Intravenous HDIT101 treatment of chronic recurrent genital HSV-2 infections leads to substantial prolongation of time to first lesion recurrence

Shown is the Kaplan Meier curve for the probability to remain lesion-free over time after treatment for MATCH-2 study participants with HSV-2 confirmed lesions either treated with Valacyclovir or with intravenous HDIT101. Starting at approximately 30 days after treatment the probability of HDIT101 treated patients to remain lesion-free was substantially increased as compared to the valacyclovir treatment arm.

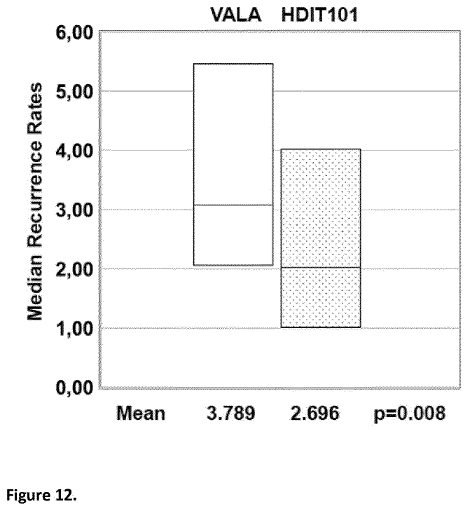

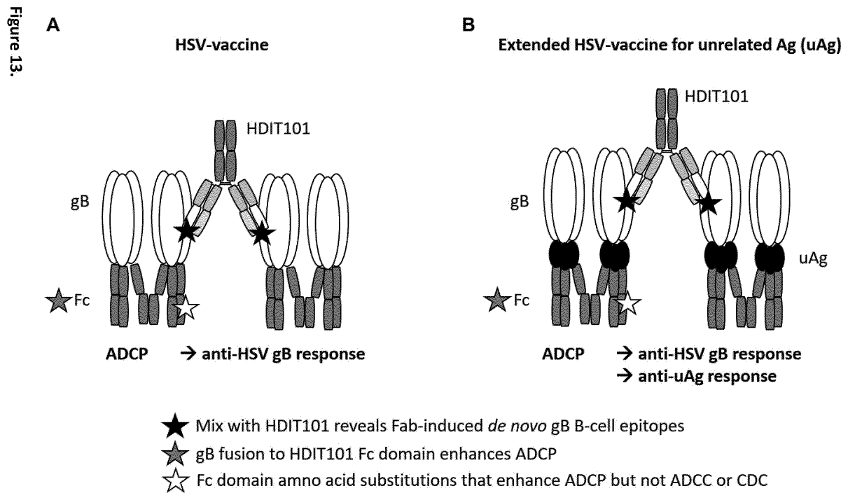

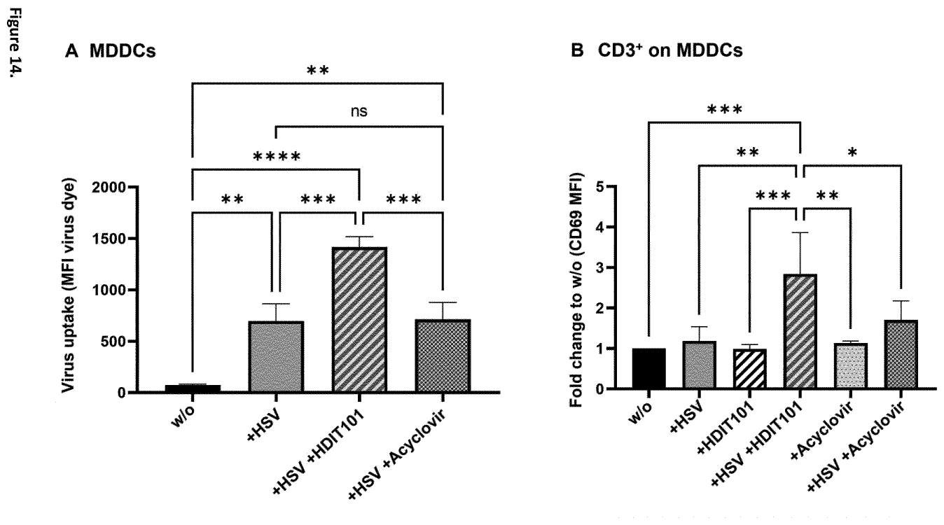

FIG. 12: Intravenous HDIT101 treatment of chronic recurrent genital HSV-2 infections leads to significant reduction in recurrence rate

Median recurrence rates and lower and upper percentiles of patients of the per protocol set are depicted. The mean recurrence rate was reduced in the per protocol set (PPS) for HDIT101 treated patients as compared to valacyclovir treated patients significantly from 3.789 to 2.696 (p=0.008).

FIG. 13: Strategy to use HDIT101: gB-Fc immune complexes mediating ADCP as a vaccine to induce anti-HSV responses or responses against unrelated Ag
(A) An HSV-1/2 gB-Fc fusion protein is generated, which forms complexes of trimers with Fc-mediated inter-trimer linkages, generating large complexes with multivalent Fc effector domains. In addition, HDIT101 enhances cross-linking, and may reveal novel B-cell epitopes due to structural changes by Fab-binding. Multivalent Fc domains from gB-Fc and HDIT101 ICs increase ADCP and anti-HSV responses, i.e. gB peptide presentation on MHC of APCs. Addition of further ADCP- but not CDC- or ADCC-, enhancing amino acid substitutions in Fc will increase vaccination effects.
(B) Same as A), however including an unrelated Ag fused between gB and Fc, to which a potent MHC T-cell response is sought to be induced. Increased Fc-effector function of ADCP due to multiple Fc-domains leads to better uptake by APCs e.g. dendritic cells, including follicular dendritic cells, which carry ICs to germinal centres where novel B-cell responses can develop. De novo T-cell responses are induced due to more efficient uptake via ADCP in APCs and more efficient presentation of ADCP-induced viral peptides on MHC. The gB-Fc:HDIT101 ICs with enhanced ADCP and MHC-presentation features can be used to deliver unrelated Ags (uAg) into the same pathway which will generate enhanced T-cell and B-cell responses against uAg.

FIG. 14: Enhanced phagocytosis and T cell activation by primary human monocyte-derived dendritic cells (MDDCs) in the presence of HDIT101

MDDCs were generated from PBMCs of independent donors by isolation of CD14 positive monocytes and differentiation using interleukin 4 (IL-4) and granulocyte-macrophage colony-stimulating factor (GM-CSF) for 7 days.
(A) Phagocytosis assay of MDDCs. MDDCs from three independent healthy donors were matured using 0.5 μg/ml lipopolysaccharide (LPS) overnight. Cells were then incubated overnight with ATTO488 NHS-Ester labeled HSV-1 at an MOI of 10 in combination with either HDIT101 (150 μg/ml), Acyclovir (50 μg/ml) or without treatment and analysed by flow cytometry the next day. Mean data for three independent donors are shown with error bars indicating standard deviations. Phagocytosis of labeled virus was increased significantly (p<0.001) in the presence of HDIT101 as compared to controls. Statistical analysis was done using one-way ANOVA test.
(B) Autologous T cell activation after ADCP by HDIT101. Same as in A) however with four independent HSV-1 seropositive healthy donors and without maturation of MDDCs. Changes in CD69 MFI were normalized to untreated MDDCs. Autologous T cells were added to the MDDCs after incubation with HSV-1 in the presence or absence of HDIT101 or Acyclovir or without treatment and analyzed by flow cytometry using fluorochrome-labeled anti-CD69 IgG as T cell activation marker. In the presence of HDIT101 the T cells showed significantly increased levels of CD69 activation marker indicating that activation was increased in the presence of HDIT101. Statistical analysis was done using one-way ANOVA test.

FIG. 15: Enhanced T cell activation by primary human monocyte-derived macrophages (MDMs) or dendritic cells (MDDCs) in the presence of HDIT101

MDMs and MDDCs were generated from PBMCs of three independent healthy donors by isolation of CD14 positive monocytes and differentiation using granulocyte-macrophage colony-stimulating factor (GM-CSF) for MDMs or interleukin 4 (IL-4)+GM-CSF for MDDCs for 7 days. Cells were incubated with HSV-1 in presence of HDIT101 or Acyclovir or untreated as above. T cell activation was determined by measuring CD69 levels on CD4+ and CD8+ cells. The data demonstrate that both, CD4+ as well as CD8+ T cells are activated to a significantly greater extent in the presence of HDIT101 in both MDMs as well as MDDCs. Statistical analysis was done using one-way ANOVA test.

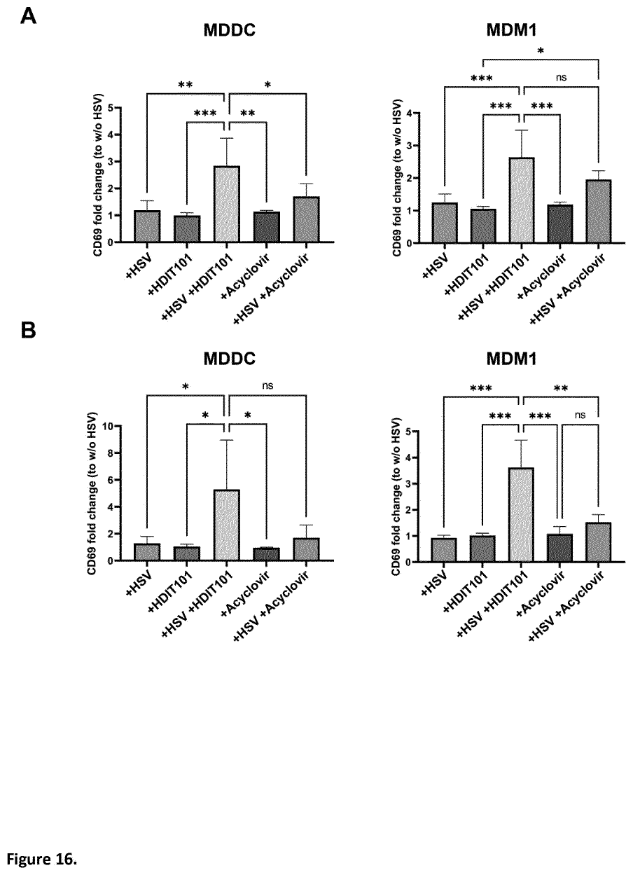

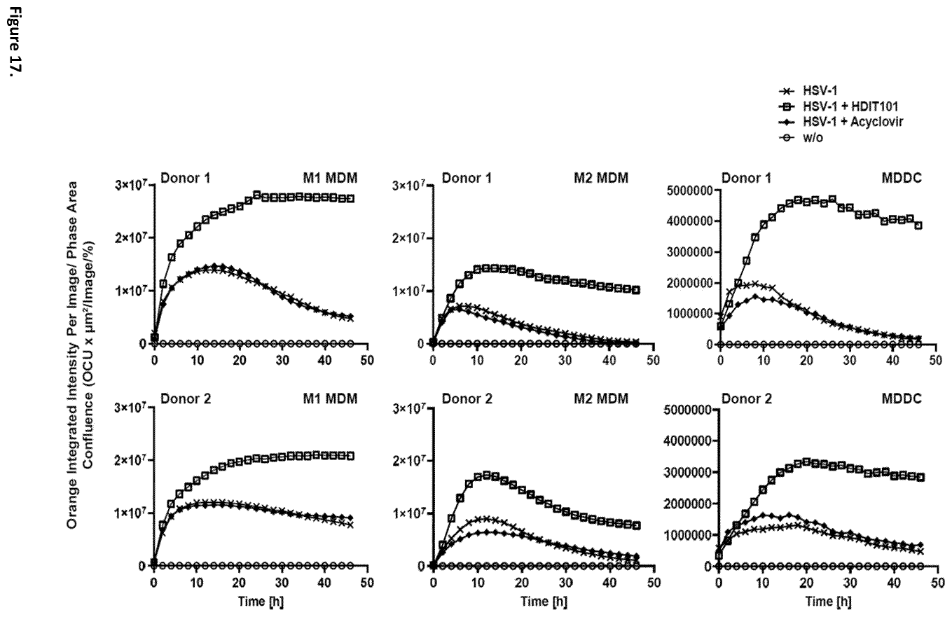

FIG. 16: Enhanced T cell activation by primary human monocyte-derived macrophages of type 1 (MDM1) or dendritic cells (MDDCs) from HSV-seropositive vs. seronegative donors in the presence of HDIT101

MDMs and MDDCs were generated from PBMCs of healthy HSV-1-seropositive (A) or HSV-seronegative (B) donors by isolation of CD14 positive monocytes and differentiation using granulocyte-macrophage colony-stimulating factor (GM-CSF) for MDMs or interleukin 4 (IL-4)+GM-CSF for MDDCs for 7 days. Cells were incubated with HSV-1 in presence of HDIT101 or Acyclovir or untreated as above. T cell activation was determined by measuring CD69 levels on CD3+ cells. Statistical analysis was done using two-way ANOVA.

FIG. 17: Rapid kinetics of ADCP-mediated by HDIT101

Monocytes were isolated from PBMCs by MACS CD14+ MicroBead separation (Miltenyi Biotec) according to the manufacturer instruction. The cells were differentiated for one week by adding M-CSF (50 ng/ml) (MDM2 cells), GM-CSF (80 ng/ml) (MDM1 cells) or GM-CSF (80 ng/ml)+interleukin 4 (IL-4) (20 ng/ml) (MDDC). The differentiated cells were then exposed to HSV-1 labeled with a pH-sensitive dye (IncuCyte pHrodo Orange Cell Labeling Dye, Sartorius) at an MOI of 10 with or without the addition of HDIT101 and/or H4 antibody at a total concentration of 150 μg/ml or Aciclovir (50 μg/ml). Three technical replicates each were then monitored using an Incucyte system (Sartorius) at intervals of 1 h. The amount of taken up virus was normalized to the cell count in the images area.

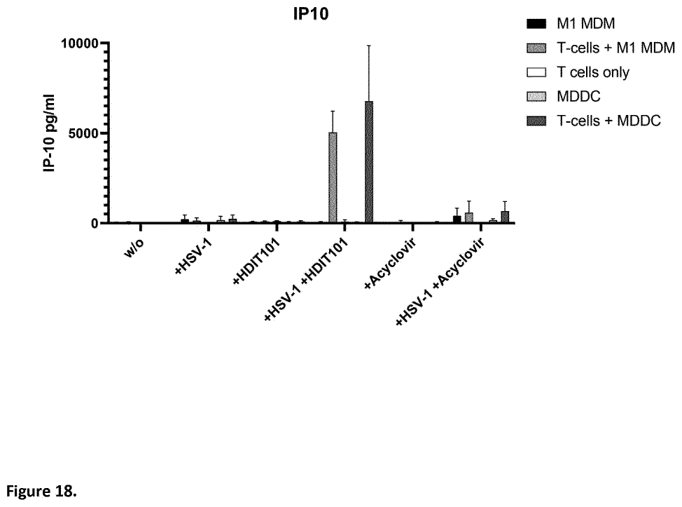

FIG. 18: HDIT101-HSV immune complexes stimulate IP10 production in the presence of MDMs or MDDCs and T cells

MDMs and MDDCs were generated from PBMCs of three independent healthy HSV-seropositive donors by isolation of CD14 positive monocytes and differentiation using granulocyte-macrophage colony-stimulating factor (GM-CSF) for MDM1 or interleukin 4 (IL-4)+GM-CSF for MDDCs for 7 days. Cells were treated overnight either with HSV-1, HDIT101, HSV-1+HDIT101, Acyclovir, or Acyclovir+HSV-1 and autologous T cells were added. The supernatants were harvested after 24 hours and subjected to IP10 measurement using LEGENDPlex™. In the presence of HDIT101+HSV-1 IP10 levels increased significantly, only in the presence of APC (either MDM or MDDC) and T cells.

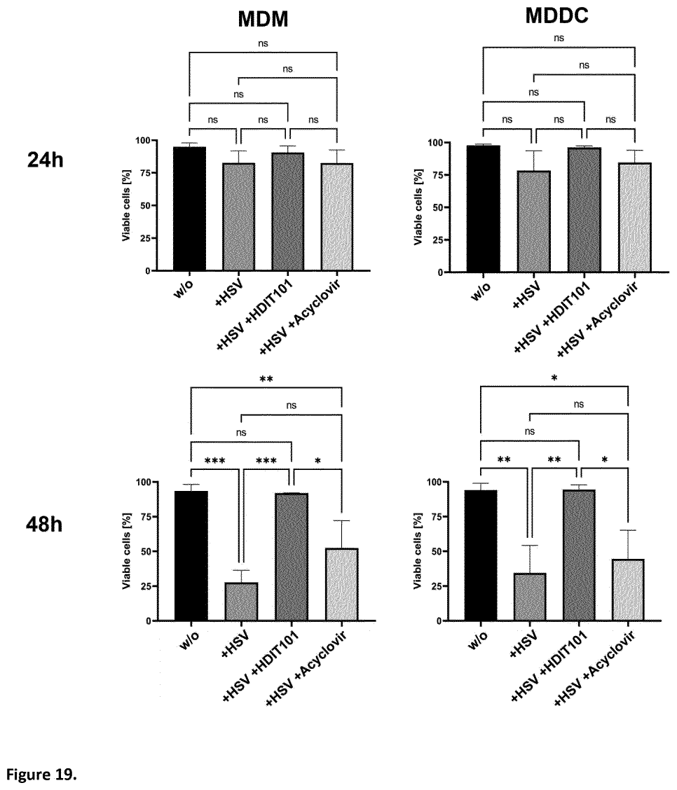

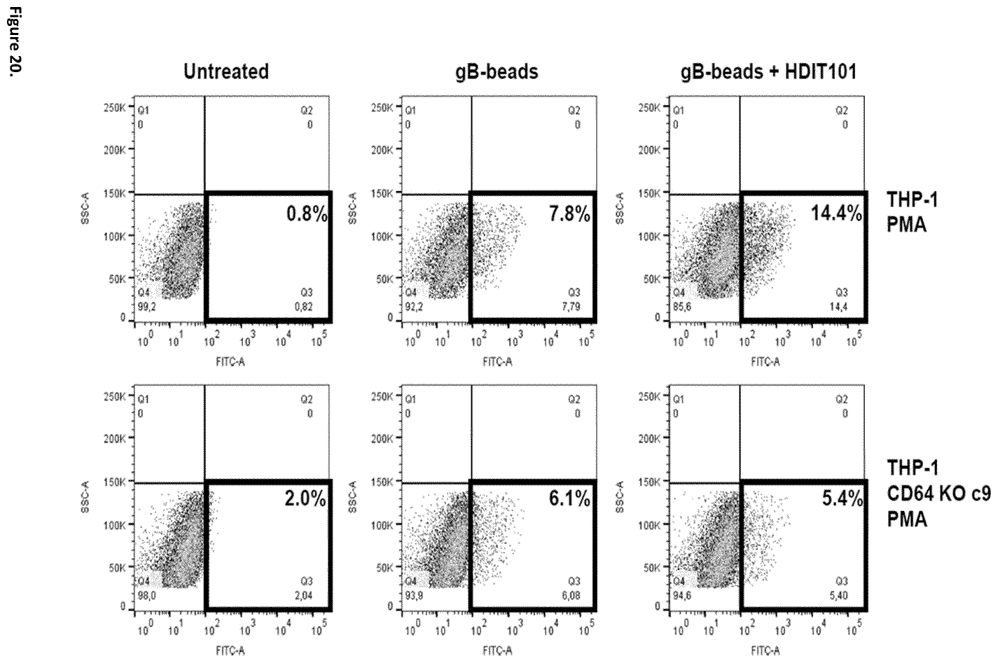

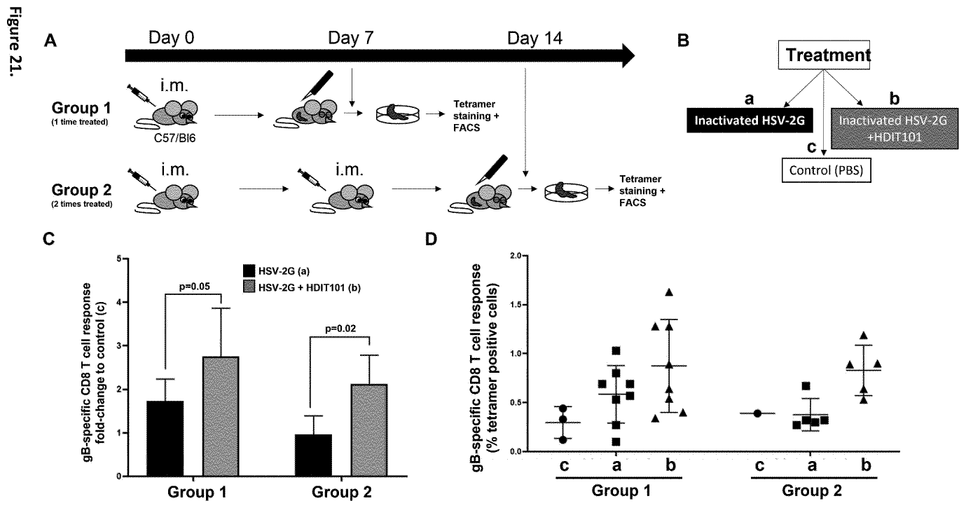

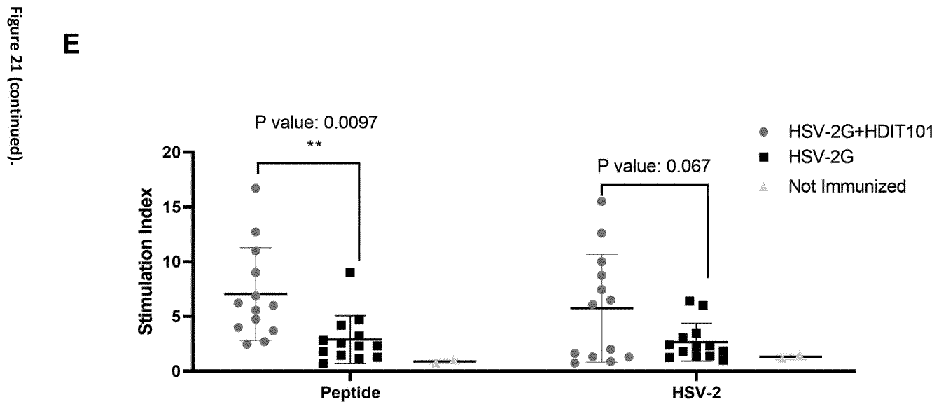

FIG. 19: Reduced cell death of primary human monocyte-derived macrophages (MDMs) or dendritic cells (MDDCs) in the presence of HDIT101

Matured MDMs or MDDCs were incubated with HSV-1 in the presence or absence of HDIT101 or Acyclovir and cell death was determined at 24 h or 48 h post treatment using Zombie-dye staining and flow cytometry for three independent donors. At 24 h post treatment no significant difference in cell death was observed. By 48 h post treatment a significantly larger proportion of cells had undergone cell death in the absence of HDIT101, even in the presence of Acyclovir, while no cell death was observed when HDIT101 was present. The data suggest that HDIT101 counteracts virus-induced cell death thereby promoting efficient T cell activation mediated by the APCs. Statistical analysis was done using one-way ANOVA test.

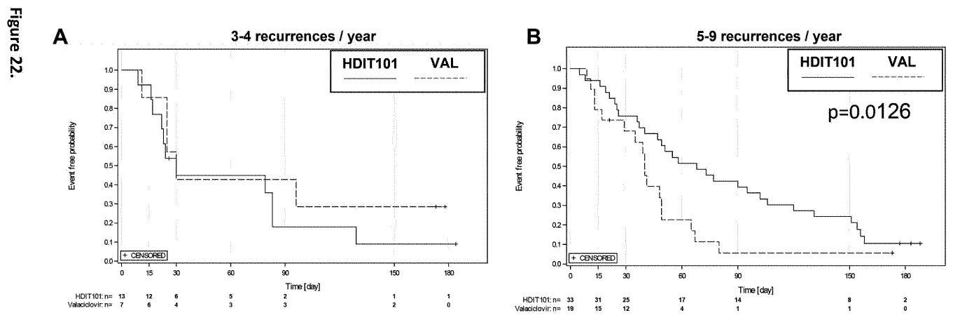

FIG. 20: FcgRI (CD64) is required for HDIT101-mediated ADCP

Phorbol-12-myristate-13-acetate (PMA) differentiated parental THP-1 cells or a CD64 knock-out (KO) clone (c9) with homozygous disruption of the CD64 open reading frame were incubated with HSV-1 gB-coated fluorescent beads in the presence or absence of HDIT101. Phagocytosis was measured by flow cytometry. HDIT101-induced ADCP was absent in CD64 knock-out cells, indicating that FcgRI (CD64) is required for the HDIT101-induced ADCP and T cell activation.

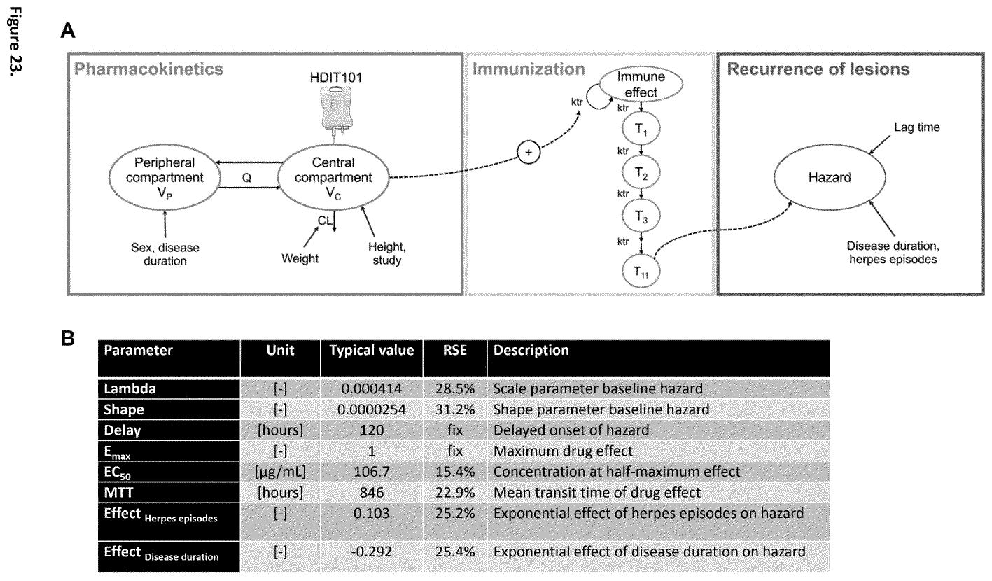

FIG. 21: HDIT101-HSV-2G immune complexes demonstrate T cell activation in mice and present a vaccine candidate
(A) Schematic of the animal experiment.
(B) Treatment arms.
(C) C57/BI-6 mice were treated intramuscularly (i.m.) with either psolaren+UV inactivated HSV-2G (a) or inactivated HSV-2G in presence of HDIT101 (b) either one time (group 1) or two times (group 2) and spleens were isolated 7 or 14 days after treatment. Splenocytes were separated and stained with a fluorophore tagged tetramer that encodes a known H-2 kB gB-specific dominant HSV-T cell epitope (SSIEFARL). Splenocytes were stained with gB-specific tetramer and analyzed by flow cytometry. The data show statistically significant increase in gB-tetramer-specific T cells in the mice after treatment with HSV-2G in the presence of HDIT101. The data indicates that HDIT101 can bind and neutralize HSV-2G and can induce an increase in activation of T cell and enhanced anti-HSV immune responses resembling a vaccine effect. Statistical analysis was done using two-way ANOVA.
(D) Individual mouse data for combined analysis in C)
(E) IFN-gamma ELISpot using splenocytes from treated mice. Splenocytes were isolated and stimulated with either gB peptide or inactivated HSV-2 and IFN-gamma secretion was measured using ELISpot (AID, Germany). Statistically significant increase in IFN-gamma secretion was observed when cells were stimulated with gB-peptide. A similar trend was observed when stimulated with HSV-2. Statistical analysis was done using two-way ANOVA.

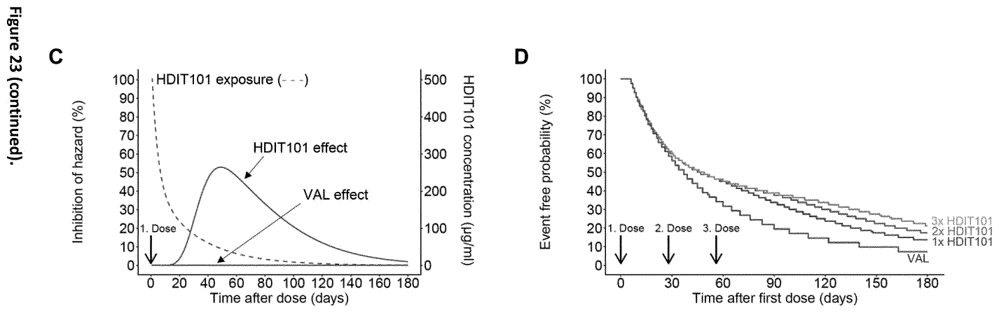

FIG. 22: Time to first recurrence in MATCH-2 study by self-reported history of recurrences

The graphs show time to event (recurrence) in Kaplan-Meier curves after treatment with HDIT101 (solid line) or first treatment with valacyclovir (dashed line) in the control arm over the time of the MATCH-2 clinical phase 2 study for individual patient subsets with self-reported history of A) 3-4, or B) 5-9 recurrences per year. A statistically significant difference was observed between treatment arms with respect to the time to first recurrence for the subset of patients with a self-reported history of 5-9 recurrences per year in the per-protocol set (VAL 44.2 days, SD=37.2, vs HDIT10181.7 days, SD=57.4; p=0.0126).

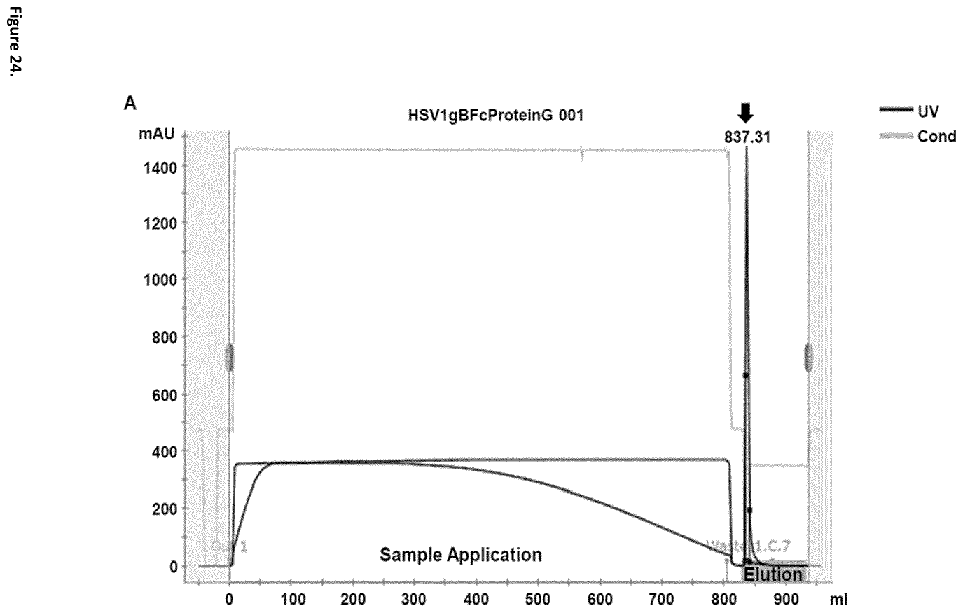

FIG. 23: PK-Time-to-event (TTE) modelling demonstrates delayed therapeutic effects in MATCH-2 and predicts improved response through multiple dosing and immune modulation
(A) Schematic representation of the PK-TTE model. The PK model of HDIT101 and the TTE model for the first recurrence of lesions are represented. Transit compartments for the delayed drug effect of HDIT101 are shown (T1-T11). Covariate effects are shown. (B) Parameter estimates with relative standard error (%) of the outcome model (time to first recurrence of lesions). (C) Simulation of HDIT101 plasma concentration-time profile with corresponding inhibitory effect on the hazard. Simulation scenario for a virtual male patient (72 kg, 174 cm, MATCH-2 study, 5 years of disease duration, 8 herpes episodes last year). The scenario fora treatment with HDIT101 or a treatment with Valacyclovir are shown. HDIT101 plasma concentration is represented with dashed lines; the inhibitory effect is represented with solid lines. (D) Simulation of multiple-dose effect of HDIT101 on median lesion-free curves over time. Simulation of one to three infusions of 2000 mg HDIT101 over one hour in comparison to 500 mg Valacyclovir over three days. Single dose administration at day 0 (1×HDIT101), multiple dose administration at day 0 and day 28 (2×HDIT101), and multiple dose administration at day 0, day 28 and day 54 (3×HDIT101) were simulated.

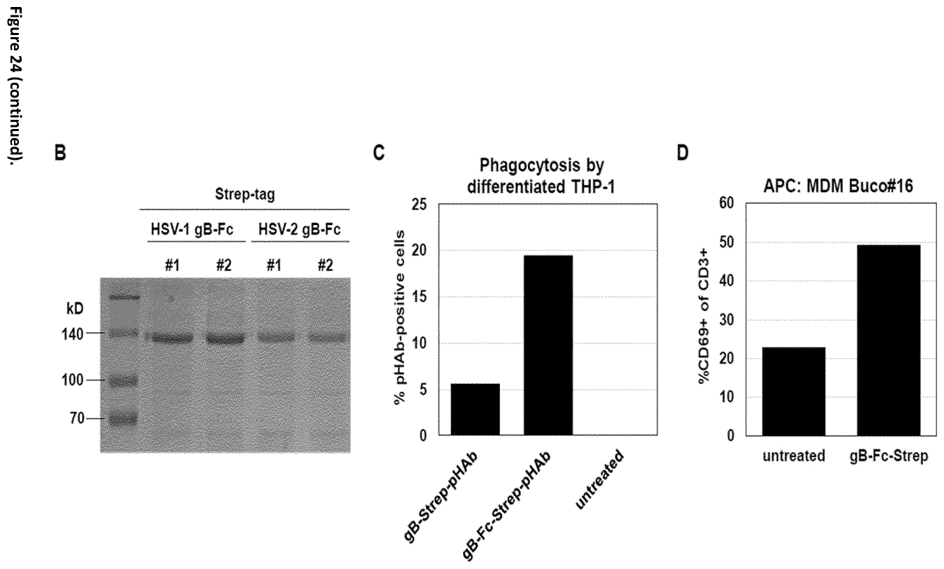

FIG. 24: HSV gB-Fc protein can be phagocytosed and can activate T cells
(A) Production and purification of gB-Fc protein using protein G affinity.
(B) Coomassie gel of streptactin-purified proteins from two independent plasmid clones for HSV-1 gB-Fc-Streptag and HSV-2 gB-Fc-Streptag.
(C) Monocytic THP-1 cells were differentiated using phorbol 12-myristate 13-acetate (PMA) (20 ng/ml) and IL-4 (20 ng/ml) for two days and matured for another day using LPS (0.5 μg/ml). HSV-1 gB-Streptag or HSV-1 gB-Fc-Streptag were labeled using a pH-sensitive amine reactive dye that leads to fluorescence after endosomal uptake. HSV-1 gB-Fc-Streptag protein was efficiently phagocytosed into low pH endosomes to a substantially greater extent as compared to HSV-1 gB-Streptag.
(D) Monocyte-derived macrophages (MDMs) were generated from buffy coats (Buco) donor #16 as antigen-presenting cells (APCs) and incubated with HSV-1 gB-Fc-Streptag protein overnight or left untreated. Autologous T cells were added and analyzed by flow cytometry for CD69 activation marker expression 24 hours later. The percentage of CD69+ cells of the CD3+ population (T cells) was calculated. The data suggest that gB-Fc incubation of APCs induced T cell activation, indicating that gB-Fc may serve as a potential vaccine candidate.

### EXAMPLES

### Example 1: Immune Modulation Via ADCP of HDIT101:HSV Complexes

HDIT101 is a Herpes Simplex Virus gB-specific antibody that neutralizes cell-free virus as well as cell-to-cell spread of HSV. In contrast to known IgG1 molecules, HDIT101 does neither induce complement-dependent cytotoxicity (CDC) (FIG. 1), nor antibody-dependent cytotoxicity (ADCC) (FIG. 2).

However, HDIT101 efficiently induces antibody-dependent phagocytosis (ADCP) in the monocytic cell line THP-1 (FIG. 3) and in primary monocyte-derived macrophages (FIG. 4). In vitro analyses identified FcgRI as main Fc-receptor binding to HDIT101 opsonized HSV-infected cells (FIG. 5), and no binding was observed to FcgRIIIa that induces ADCC or FcgRIIa. Hence FcgRI seems to be the predominant Fc-receptor that is used by HDIT101, however a role for other non-classical FcRs, FcRn or TRIM21, cannot be excluded. HDIT101 opsonization of 293T cells infected with HSV-1 and subsequent incubation with M2-differentiated primary human monocyte-derived macrophages (MDM) resulted in ADCP and induced production of monocyte/macrophage-relevant proinflammatory cytokines (FIG. 6).

Most strikingly, levels of CXCL10 (IP10) produced in MDMs from one donor were increased by ICs of infected cells complexed with HDIT101 by 1,000-fold (FIG. 6). In addition, in the presence of non-infectious HDIT101/HSV-1 viral particle ICs, but not HDIT101 alone, IP10 production was increased by 20,000-fold in a co-culture of autologous T cells with MDMs from one donor, while TNFa levels were increased by ^{˜}70-fold, IL-6 levels were increased by ^{˜}20-fold and IFNg levels were increased by ^{˜}120-fold (FIG. 7). This data shows that ICs of HDIT101 bound to HSV can stimulate cytokine responses in monocyte derived macrophages through ADCP and that the presence of T cells strongly enhances this response. When comparing the capability of M1 versus M2 type MDMs, we observed that autologous T-cell derived IL-2 production was strongly increased by HSV:HDIT101 ICs in the presence of M1-differentiated MDMs from one donor, while only moderately induced in the presence of M2-differentiated MDMs and not induced in the absence of MDMs (FIG. 8). This data suggest T-cell activation by macrophages after phagocytosis of HDIT101:HSV-1 ICs.

IP10 is usually stimulated by IFNg (i.e., IFN gamma) from T cells however can also be produced by monocytes, macrophages or dendritic cells when encountering a viral pathogen or virus infected cells. Indeed, monocyte-derived macrophages produced IP10 independently of interferon-stimulated gene IFI16 and independently of HSV-1 replication [57]. Strikingly, IP10 was essential in controlling genital infection in mice by recruiting NK and T cells. Knock-out of IP10 resulted in dramatically earlier HSV-2 detection in the central nervous system, reduced migration of NK and T cells to the spinal cord and reduced survival, demonstrating that IP10 plays a critical role in controlling spread of infection [58]. In addition, IP10 was also shown to mobilize HSV-specific CD8+ T effector memory as well as tissue-resident CD8+ T memory cells [59]. Moreover, IP10 serves as chemotactic agent also for dendritic cells since IP10 knock-out reduced migration of dendritic cells to sites of HSV-1 infection [60].

Given that ICs of HSV-1 infected 293T cells complexed with HDIT101 showed strong IP10 induction in MDM, the present data propose invention that a key feature of establishing de novo T cell responses by HDIT101 treatment through ADCP is the induction of IP10 which helps recruiting NK and T cells to the site of phagocytosis of opsonized infected cells or virus by APCs. At the same time dendritic cells are attracted due to HDIT101:IC-induced IP10 production, leading to more ADCP and further IP10 production, acting like a positive feedback loop strongly enhancing the cellular immune response (FIG. 9). The present data propose that IP10 induction by HDIT101 opsonized infected cells or virus and effects on enhanced activation of existing and de novo T cell responses rely on the feature of HDIT101 to mediate ADCP without inducing CDC or ADCC. This feature forms the rationale for the vaccine design of the present invention in that virus particles or virus like particles (VLPs) with glycoprotein B (gB) or recombinant gB or gB-Fc fusion proteins can be bound by HDIT101 and these ICs can be used for vaccination (FIG. 9).

Indeed, the Fc effector domain is essential for the mode of action of HDIT101 in vivo. When immunocompetent Balb/c mice were intravaginally infected with HSV-2G, and treated thereafter with either HDIT101 or HDIT101 N297A, survival was much reduced in the HDIT101N297A treated group. Treatment with HDIT101 or murine mAb2c increased survival within 30 days to 50%, while all control mice died within 10 days. HDIT101 N297A treated mice showed significantly reduced survival as compared to HDIT101 treated mice (p=0.0036, Gehan-Breslow-Wilcoxon statistical test) (FIG. 10).

Clinical data shows that HDIT101 treatment prolonged the time to first recurrence of lesions (FIG. 11), an effect that could be explained by an active immunization. Intriguingly, the probability for developing a lesion after treatment was almost identical for the HDIT101 and valacyclovir groups until approximately day 30 after treatment, when the Kaplan-Meier curve of the HDIT101-treated group changes slope and the probability of developing a lesion is reduced as compared to the valacyclovir arm. The bi-phasic course of the probability to develop a lesion when HDIT101 treated suggests that immune modulation takes place within 30 days after treatment and the protective effects are exerted thereafter (FIG. 11). In addition, the mean recurrence rate was reduced in the per protocol set (PPS) for HDIT101 treated patients as compared to valacyclovir treated patients significantly from 3.789 to 2.696 (p=0.008) (FIG. 12).

From the present data, it is proposed that HDIT101 binds to HSV particles or HSV infected cells generating ICs that are efficiently taken up by APCs via ADCP. Phagocytosis leads to direction of ICs for degradation within the lysosome by e.g. by cysteine proteases such as cathepsins S and direct loading on MHC-I molecules (vacuolar pathway) or by uptake of Ag or partially digested Ag into the cytoplasm and degradation via the proteasome (proteasomal pathway). Induction of IP10 production by APCs attracts T cells and monocytes, macrophages and dendritic cells. The specific features of HDIT101, i.e. the absence of induction of ADCC and CDC, and binding properties to its target protein gB allow for uptake, and proteolytic cleavage in a way that the efficiency of viral peptide presentation on MHC class II for CD4+ T cell stimulation as well as on MHC class I via cross-presentation for CD8+ T cell stimulation is enhanced.

In addition, HDIT101 directs via follicular dendritic cells gB-containing ICs towards germinal centres were IC-specific de novo B-cell epitopes stimulate the activation and maturation of B cells specific for HDIT101-induced gB epitopes. Together these de novo T cell and B cell responses lead to a long-lasting protection from reactivation of latent HSV and the reduction of lesion rates and the prolongation of the time to the first HSV lesion recurrence after treatment.

### Example 2: Use of HSV-1/2 gB-Fc Fusion Protein Complexed with gB-Specific Antibodies as Vehicle to Induce De Novo B- and T-Cell Responses

The rational of an induction of de novo T cell and B cell responses to induce long-lasting protection from HSV lesion recurrences due to the features of HDIT101 to not induce ADCC or CDC combined with its binding properties to its target protein gB, led to the proposal to combine the effects to generate a vaccine.

This vaccine can either comprise whole virus particles that are complexed with HDIT101 or trimeric gB ectodomain fused to the Fc portion of HDIT101 complexed in addition with HDIT101.

ADCP can even be further enhanced by introduction of specific amino acid substitutions in Fc. As an Fc-fusion protein, gB-Fc will adopt trimeric structures that will contain 3 Fc domains, two of which will dimerize, leaving one free Fc domain that can dimerize with free Fc domains of other gB-Fc trimers generating larger complexes of multiple copies of gB-Fc. To increase immunogenicity, to reveal HDIT101-binding induced novel epitopes and to direct the structures towards ADCP, HDIT101 will be mixed with gB-Fc protein units leading to multimeric complexes of gB-Fc protein multivalently bound to HDIT101 molecules. These structures will combine the feature of the gB-Fc Ag target and the properties of HDIT101 for enhanced ADCP, while importantly avoiding ADCC and CDC induction, which should lead to a largely increased T-cell (and B-cell) response and vaccinal effects. It is proposed that this mAb-enhanced IC can be used as vehicle for enhanced delivery of unrelated Ags into the ADCP pathway, leading to the activation of de novo T-cell (and B-cell) responses (FIG. 13).

### Example 3: Dendritic Cells Phagocytose HDIT101:HSV Immune Complexes and Strongly Activate T Cell Responses

Dendritic cells play important roles as APCs by phagocytosing antibody opsonized pathogens and stimulating T cell responses. HDIT101-opsonized immune complexes are significantly more efficiently taken up by monocyte-derived dendritic cells (MDDCs) as virus without antibody (FIG. 14 A) and in addition significantly better activated autologous T cells (FIG. 14 B). Enhanced CD69 activation responses were seen for both CD4+ and CD8+ T cells (FIG. 15) and were independent of the HSV-1/2-serostatus (FIG. 16). The kinetics of ADCP for HDIT101:HSV-1 immune complexes was similar for MDDCs as compared to M1 and M2 MDMs as measured by uptake of pH-sensitive dye-labelled immune complexes over a time course of 48 hours (FIG. 17). This indicates that all three types of APCs M1 and M2 macrophages, as well as dendritic cells, can contribute to ADCP-induced T cell stimulation by HDIT101 and shows rapid kinetics of ADCP mediated by HDIT101.

### Example 4: HDIT101:HSV-1 Immune Complexes Strongly Induce IP-10 in the Presence of Dendritic Cells and T Cells

As shown, levels of CXCL10 (IP10) produced in MDMs were increased by immune complexes (ICs) of HDIT101 bound to infected cells (FIG. 6) or HSV-1 viral particles (FIG. 7), but not by HDIT101 alone, in the presence of T cells. Indeed, IP10 expression was also dramatically increased, when monocyte-derived dendritic cells (MDDCs) from three independent donors were incubated with HSV-1:HDIT101 ICs, to a slightly higher extent as seen with M1 MDMs (FIG. 18). IP10 was only produced in the presence of M1 MDMs or MDDCs and T cells, but not when only T cells or only M1 MDMs or MDDCs were present. IP10 is usually stimulated by IFNg from T cells however can also be produced by monocytes, macrophages or dendritic cells when encountering a viral pathogen or virus infected cells. Indeed, monocyte-derived macrophages produced IP10 independently of interferon-stimulated gene IFI16 and independently of HSV-1 replication [57]. Strikingly, IP10 was essential in controlling genital infection in mice by recruiting NK and T cells. Knock-out of IP10 resulted in dramatically earlier HSV-2 detection in the central nervous system, reduced migration of NK and T cells to the spinal cord and reduced survival, demonstrating that IP10 plays a critical role in controlling spread of infection [58]. In addition, IP10 was also shown to mobilize HSV-specific CD8+ T effector memory as well as tissue-resident CD8+ T memory cells [59]. Moreover, IP10 serves as chemotactic agent also for dendritic cells since IP10 knock-out reduced migration of dendritic cells to sites of HSV-1 infection [60].

The present data propose that a key feature of establishing de novo T cell responses by HDIT101 treatment through ADCP is the induction of IP10 which helps recruiting NK and T cells to the site of phagocytosis of opsonized infected cells or virus. At the same time dendritic cells are attracted due to HDIT101:IC-induced IP10 production, leading to more ADCP and further IP10 production (FIG. 18), acting like a positive feedback loop strongly enhancing the cellular immune response (FIG. 9). The new data on IP10 production by HDIT101-induced phagocytosis of virus in presence of MDMs/MDDCs and T cells strongly supports the perpetuating model of IP10 production to recruit immune cells until all virus and infected cells have been cleared. This includes de novo recruitment of T cells as well as more APCs (DCs, MDMs), NK cells and B cells.

### Example 5: HDIT101 Protects APCs During an Acute HSV Infection from Cell Death Thereby Ensuring Efficient T Cell Stimulation

A prerequisite for an efficient T cell stimulation is the ADCP-induced uptake of HDIT101:HSV immune complexes and at least for MHC-mediated T cell stimulation the presentation of viral peptides on the MHC complex. The key for a prolonged T cell activation therefore is the survival of the APCs. HDIT101 treatment not only causes the uptake of HDIT101:HSV immune complexes, it also prevents virus-induced death, substantially better than standard of care treatment drug acyclovir. Therefore, HDIT101-opsonized HSV immune complexes not only stimulated T cell responses after ADCP, HDIT101 treatment also prevented MDM and MDDC cell death, a further mechanism to induce enhanced long-term persisting T cell stimulation via promoting better survival of APCs (FIG. 19).

### Example 6: HDIT101 Mediates ADCP Via Interaction with FcgRI (CD64)

To investigate which FcgR is required for the ADCP and T cell activation, THP-1 cells were subjected to gene editing to knock-out FcgRI (CD64) and a knock-out cell clone (c9) with a homozygously disrupted CD64 gene was identified and subjected for HDIT101-induced ADCP measurements of HSV-1 gB coated fluorescent beads. While parental cells efficiently phagocytosed HDIT101 opsonized gB-coated beads, this was not the case for THP-1 CD64 knock-out cells, indicating that FcgRI (CD64) is required for HDIT101-directed ADCP and T cell stimulation (FIG. 20).

### Example 7: HDIT101:HSV-2G Immune Complexes Induce Functional Anti-HSV-2 T Cell Responses In Vivo

To demonstrate immune stimulatory T cell activation capabilities of HDIT101 in vivo, HSV-2G was inactivated using psoralen treatment and UV-illumination and C57/Bl6 mice were used as an immunization model. Per mouse 5 μl of inactivated HSV-2G (3.55*10e8 TCID50/ml) was mixed with 25 μl of HDIT101 (50 mg/ml stock) or PBS and injected intramuscularly (i.m.) according to experimental treatment scheme. 6 weeks old C57/Bl6 mice (Envigo) were acclimatized for 1 week before the first treatment. Mice in group 1 received only one time treatment and splenocytes were isolated on day 7. Mice in group 2 were treated on day 0 and day 7 and splenocytes were isolated on day 14 (FIGS. 21 A and B). Splenocytes were prepared by homogenization of spleens and erythrocyte lysis and frozen in freezing medium before further analysis by flow cytometry. For analysis splenocytes were stained with a fluorochrome-labeled tetramer resembling H-2Kb-gB peptide SSIEFARL that recognizes HSV-specific T-cells in C57/Bl6 mice (FIGS. 21 C and D). To analyze functionality of the T-cells splenocytes were stimulated ex vivo with either SSIEFARL peptide or whole inactivated HSV-2G and interferon gamma (IFNγ) responses were determined with a commercial murine IFNγ ELISpot (AID, Germany) (FIG. 21 E). In C57/Bl6 mice treated with immune complex of HSV-2G bound to HDIT101, a significantly (p=0.05 for one time treatment, group 1 and p=0.02 for two times treatment, group 2) higher percentage of splenocyte-derived T-cells specifically recognizing the gB SSIEFARL peptide tetramer were identified, as compared to animals that received inactivated HSV-2G only. When the splenocytes were tested for a functional response of T-cells by incubation with HSV gB peptide SSIEFARL a significantly (p=0.0097) greater response was observed in animals that were treated with HSV-2G in complex with HDIT101 as compared without antibody. Similar results were observed when cells were stimulated with whole inactivated HSV-2G. The data suggest that HDIT101 enhances anti-HSV-2G T cell response in mice by promoting APC-mediated uptake of HSV-2 immune complexes and subsequent T cell activation. To our knowledge this is the first time that a therapeutic antibody, for which no ADCC or CDC could be detected, but that can potently mediate ADCP, demonstrates a vaccine-like effect by enhancing T cell responses.

### Example 8: Time to First Recurrence in MATCH-2 Study by Self-Reported History of Recurrences

The analysis of key secondary efficacy endpoints in the MATCH-2 study provided an indication for a pronounced indirect beneficial effect of HDIT101. Markedly longer time-to-first-recurrence and a lower recurrence rate in favor of HDIT101 were observed (FIG. 11) and (FIG. 12). For the subgroup of patients having self-reported 5-9 recurrences in the last 12 months (50% of randomized patients) a significant difference was observed in the time-to-first-recurrence in the per-protocol set in favor of the HDIT101 arm (FIG. 22). Notably, such heavily affected patients are regularly excluded in other clinical trials employing antiviral HSV therapeutics ([65], [66])

### Example 9: An Exposure-Response Model for HDIT101 Treatment Shows Increased Long-Term Effects on Recurrence-Free Time for Multiple Dosing HDIT101 Immunization Strategy

To investigate the impact of HDIT101 exposure on the outcome, defined as the time to first recurrence of lesions, a pharmacokinetic (PK)-time to event (TTE) modeling analysis was performed.

The population PK model derived from the combined analysis of MATCH-2 and the data from the first-in-human trial (clinical study HDIT101-01 with EudraCT-Nr. 2017-004452-37) was used to describe the concentration-time profiles in patients with PK sampling and to predict the PK in patients without PK sampling based on their covariates and their respective administration protocol. Population pharmacokinetic analysis was performed by non-linear mixed-effects modeling within the software NONMEM 7.4.3 (ICON Development Solutions, Ellicott City, MD, USA). The first-order condition estimation algorithm with interaction was used for parameter estimation. For inter-individual variability (IIV) exponential random effects models were used. Model selection criteria were adequate goodness-of-fit plots, the precision of parameter estimates, and a significant reduction in NONMEM objective function value. Modeling was done in a stepwise procedure. First, a PK base model was developed by testing one-, two- and three-compartment and TMDD models. Second, laboratory baseline values (albumin, ASAT, GFR, GGT), laboratory longitudinal values (albumin, ALAT, ASAT, bilirubin, GFR, GGT, protein), and patient characteristics (BMI, BSA, disease duration, height, hormonal contraception, sex, study, weight, ADAs, smoke status) were considered as covariates.

Multivariate covariate analysis was done as forward inclusion (p≤0.01) and backward elimination (p≤0.001). Categorical covariates were tested as a factor; numerical covariates were tested as power function and linear. For time-varying laboratory values change from individual baseline values were tested, too.

With regard to exposure-response analysis, a time-to-event analysis for the endpoint ‘time to first recurrence of lesions’ was performed within the software NONMEM 7.4.3 (ICON Development Solutions, Ellicott City, MD, USA). For parameter estimation, the Laplacian method was used. Model selection criteria were adequate visual predictive check (VPC), the precision of parameter estimates, and a significant reduction in NONMEM objective function value. The parametric survival function, as given in Eq. 1, was used to analyze the time to first recurrence of lesions.

$$S ( t ) = {e}^{- {\int }_{0}^{t} h ( t ) dt} \qquad Eq . 1$$

h(t)=Hazard function for recurrence of lesion, S(t)=Probability of being lesion-free within the time interval 0 (study start) to time t.

Modeling was done stepwise. First, different baseline hazard models were evaluated, including proportional, Gompertz, and Weibull hazard functions. Additionally, a lag time for delayed onset of hazard was tested. Second, the drug effect of HDIT101 was tested using an Emax function, and direct links from HDIT101 plasma concentration to baseline hazard, via an effect compartment model and transit compartments were explored. The number of transit compartments was optimized. Third, covariate candidates (age, sex, protein, albumin, disease duration, number of anogenital herpes episodes last year, hormonal contraceptives, smoking status) were tested on the hazard function parameter (lambda and shape parameter). Multivariate covariate analysis was done as forward inclusion (p≤0.01) and backward elimination (p≤0.001). Categorical covariates were tested as a factor; numerical covariates were tested as power function and linear. For time-varying laboratory values change from individual baseline values were tested, too.

The Gompertz hazard function described the time to first recurrence best and a lag time of delayed onset of the hazard of 5 days was identified, improving the model description significantly. No direct effect of HDIT101 exposure on the baseline hazard could be identified. However, a statistically significant impact of HDIT101 on the baseline hazard was identified, if transit-compartment models were included, similar to the model of Goyal et al., which used transit compartments for a delayed immune effect initiated by SARS-Cov2-virus load ([67]). In our model, 11 transit-compartments described the exposure-response relationship best and resulted in an estimated mean transit time (MTT) of 35 days (FIG. 23 A). The effect of valacyclovir on the hazard was also investigated using a published PK model for valacyclovir ([68]), to mimic the exposure in valacyclovir treated patients. Overall, no significant effect of valacyclovir exposure on the baseline hazard could be identified. All parameters were estimated precisely (relative standard error (RSE) <40%) (FIG. 23 B) and the model code including all differential equations is provided on GitHub (https://github.com/Clinical-Pharmacy-Saarland-University).

To illustrate the effect of HDIT101 on the hazard, the pharmacokinetic-outcome relationship of a typical patient (72 kg, 174 cm, male, 5 years of disease duration and 8 episodes in the last year) receiving 2000 mg of HDIT101 as infusion was simulated. The respective HDIT101 concentration-time profile and the percentage of inhibition of the hazard are shown in (FIG. 23 C). The HDIT101 exposure declines exponentially as expected, and the drug effect starts to be active after approximately 20 days and the maximum drug effect achieved after 49 days resulting in a 53% decrease of the hazard. The HDIT101 drug effect lasts for more than 100 days, where still a more than 10% reduction in the hazard can be identified. This is also reflected in the cumulative hazard, which separates after about 30 days and has a different shape up to 120 days under HDIT101 treatment compared to valaciclovir treatment.

A covariate analysis identified that the number of anogenital herpes episodes a patient had in the last year and the disease duration of the patient had a significant impact (p<0.01) on the baseline hazard. A larger number of anogenital episodes in the last year in patients resulted in a higher hazard and a faster onset of recurrence.

To extrapolate the drug effect from single dose administration of HDIT101 to multiple dose administration, the median probability of being lesion-free for three different scenarios were simulated. Simulation scenarios were (I) single dose administration of HDIT101 on day 0, (II) multiple dose administration of HDIT101 on day 0 and day 28 and (III) multiple dose administration of HDIT101 on day 0, day 28 and day 54 (FIG. 23 D). Simulated patients treated with two and three doses of HDIT101 show higher median probabilities of being lesion-free after day 68 and day 95 compared to one and two doses of HDIT101, respectively.

### Example 10: HSV gB-Fc Proteins can be Phagocytosed and can Activate T Cells

In order to demonstrate that a gB-Fc fusion protein consisting of the HSV gB extracellular domain artificially fused at its carboxy-terminus to the amino-terminus of the hinge-CH2-CH3 moiety (Fc domain) of HDIT101 has the potential to elucidate immunomodulatory effects, HSV gB-Fc fusion proteins (SEQ. ID 74 to 77) were synthesized by transient transfection of HEK293T cells and purified via affinity chromatography either with protein G (FIG. 24 A) or in case strep-tagged proteins were used with streptactin. Coomassie-gel analysis of purified proteins showed a high degree of purity with little background (FIG. 24B). The generated strep-tagged HSV gB-Fc fusion protein was tested in phagocytosis assays by coupling the protein to pH-sensitive fluorescent beads that become fluorescent upon endocytosis. Phagocytosis was measured in the monocytic cell line THP-1 and demonstrated enhanced capability of HSV gB-Fc-Streptag labelled beads to be phagocytosed as compared to gB-Streptag (SEQ. ID 78) labelled beads (FIG. 24C), suggesting functionality to be taken up by antigen-presenting cells. Indeed, incubation of primary MDM of one donor with HSV gB-Fc-Streptag protein and subsequent co-incubation with autologous T cells resulted in enhanced T-cell activation as measured by CD69 expression using flow cytometry (FIG. 24D). The data demonstrate the potential of HSV gB-Fc to be further optimized and developed into a vaccine.

The Sequences Used and/or Exemplified in this Example are Briefly Described in the Following:
HSV-1F gB extracellular domain fused to IgG1 hinge*-CH2-CH3 (SEQ ID NO: 74)
HSV-2G gB extracellular domain fused to IgG1 hinge*-CH2-CH3 (SEQ ID NO: 75)
HSV-1F gB extracellular domain fused to IgG1 hinge*-CH2-CH3-StrepTag (SEQ ID NO: 76)
HSV-2G gB extracellular domain fused to IgG1 hinge*-CH2-CH3-StrepTag (SEQ ID NO: 77)
HSV-1F gB extracellular domain fused to StrepTag (SEQ ID NO: 78)
HSV-2G gB extracellular domain fused to StrepTag (SEQ ID NO: 79)

The strep-tagged sequences have also a thrombin cleavage site before the StrepTag to remove it.

### REFERENCES

1. Pollard, A. J. and E. M. Bijker, A guide to vaccinology: from basic principles to new developments. Nat Rev Immunol, 2021. 21(2): p. 83-100.
2. Evert, B. J., et al., Epitope-coated polymer particles elicit neutralising antibodies against Plasmodium falciparum sporozoites. NPJ Vaccines, 2021. 6(1): p. 141.
3. Pardi, N., et al., mRNA vaccines—a new era in vaccinology. Nat Rev Drug Discov, 2018. 17(4): p. 261-279.
4. Mallapaty, S., India's DNA COVID vaccine is a world first—more are coming. Nature, 2021. 597(7875): p. 161-162.
5. Kato, Y., et al., Multifaceted Effects of Antigen Valency on B Cell Response Composition and Differentiation In Vivo. Immunity, 2020. 53(3): p. 548-563 e8.
6. Junker, F., J. Gordon, and O. Qureshi, Fc Gamma Receptors and Their Role in Antigen Uptake, Presentation, and T Cell Activation. Front Immunol, 2020. 11: p. 1393.
7. Alcaide, E. G., S. Krishnarajah, and F. Junker, Dendritic Cell Tumor Vaccination via Fc Gamma Receptor Targeting: Lessons Learned from Pre-Clinical and Translational Studies. Vaccines (Basel), 2021. 9(4).
8. Duivelshof, B. L., et al., Therapeutic Fc-fusion proteins: Current analytical strategies. J Sep Sci, 2021. 44(1): p. 35-62.
9. Sun, S., et al., Recombinant vaccine containing an RBD-Fc fusion induced protection against SARS-CoV-2 in nonhuman primates and mice. Cell Mol Immunol, 2021. 18(4): p. 1070-1073.
10. Loureiro, S., et al., Adjuvant-free immunization with hemagglutinin-Fc fusion proteins as an approach to influenza vaccines. J Virol, 2011. 85(6): p. 3010-4.
11. Zhao, B., et al., Immunization With Fc-Based Recombinant Epstein-Barr Virus gp350 Elicits Potent Neutralizing Humoral Immune Response in a BALB/c Mice Model. Front Immunol, 2018. 9: p. 932.
12. Konduru, K., et al., Ebola virus glycoprotein Fc fusion protein confers protection against lethal challenge in vaccinated mice. Vaccine, 2011. 29(16): p. 2968-77.
13. Ye, L., et al., Efficient mucosal vaccination mediated by the neonatal Fc receptor. Nat Biotechnol, 2011. 29(2): p. 158-63.
14. Botelho, N. K., et al., Combination of Synthetic Long Peptides and XCL1 Fusion Proteins Results in Superior Tumor Control. Front Immunol, 2019. 10: p. 294.
15. Wiedinger, K., J. McCauley, and C. Bitsaktsis, Isotype-specific outcomes in Fc gamma receptor targeting of PspA using fusion proteins as a vaccination strategy against Streptococcus pneumoniae infection. Vaccine, 2020. 38(35): p. 5634-5646.
16. Rodriguez-de la Noval, C., et al., Protective Efficacy of Lectin-Fc(IgG) Fusion Proteins In Vitro and in a Pulmonary Aspergillosis In Vivo Model. J Fungi (Basel), 2020. 6(4).
17. <WO2017081066A1_immunoassembline Malaria.pdf>.
18. Kranich, J. and N. J. Krautler, How Follicular Dendritic Cells Shape the B-Cell Antigenome. Front Immunol, 2016. 7: p. 225.
19. Heesters, B. A., et al., Endocytosis and recycling of immune complexes by follicular dendritic cells enhances B cell antigen binding and activation. Immunity, 2013. 38(6): p. 1164-75.
20. Manca, F., et al., Differential activation of T cell clones stimulated by macrophages exposed to antigen complexed with monoclonal antibodies. A possible influence of paratope specificity on the mode of antigen processing. J Immunol, 1988. 140(9): p. 2893-8.
21. <Manca et al. 1991 AgAb ratio effects on T cell stimulation and Ag presentation.pdf>.
22. Ebenezer, R. S., et al., Protective effect of antigen excess immune complex in guinea pigs infected with Mycobacterium tuberculosis. Indian J Med Res, 2017. 146(5): p. 629-635.
23. Xu, D. Z., et al., Vaccination with recombinant HBsAg-HBIG complex in healthy adults. Vaccine, 2005. 23(20): p. 2658-64.
24. Zhou, C., et al., Analysis of immunological mechanisms exerted by HBsAg-HBIG therapeutic vaccine combined with Adefovir in chronic hepatitis B patients. Hum Vaccin Immunother, 2017. 13(9): p. 1989-1996.
25. Xu, D. Z., et al., A randomized controlled phase IIb trial of antigen-antibody immunogenic complex therapeutic vaccine in chronic hepatitis B patients. PLoS One, 2008. 3(7): p. e2565.
26. Li, J., et al., Research progress of therapeutic vaccines for treating chronic hepatitis B. Hum Vaccin Immunother, 2017. 13(5): p. 986-997.
27. Bournazos, S. and J. V. Ravetch, Fcgamma receptor pathways during active and passive immunization. Immunol Rev, 2015. 268(1): p. 88-103.
28. Haigwood, N. L., et al., Passive immunotherapy in simian immunodeficiency virus-infected macaques accelerates the development of neutralizing antibodies. J Virol, 2004. 78(11): p. 5983-95.
29. Ng, C. T., et al., Passive neutralizing antibody controls SHIV viremia and enhances B cell responses in infant macaques. Nat Med, 2010. 16(10): p. 1117-9.
30. Nishimura, Y., et al., Early antibody therapy can induce long-lasting immunity to SHIV. Nature, 2017. 543(7646): p. 559-563.
31. Tsouchnikas, G., et al., Immunization with Immune Complexes Modulates the Fine Specificity of Antibody Responses to a Flavivirus Antigen. J Virol, 2015. 89(15): p. 7970-8.
32. Tang, A. F., G. Enyindah-Asonye, and C. E. Hioe, Immune Complex Vaccine Strategies to Combat HIV-1 and Other Infectious Diseases. Vaccines (Basel), 2021. 9(2).
33. Rawool, D. B., et al., Utilization of Fc receptors as a mucosal vaccine strategy against an intracellular bacterium, Francisella tularensis. J Immunol, 2008. 180(8): p. 5548-57.
34. Schuurhuis, D. H., et al., Immune complex-loaded dendritic cells are superior to soluble immune complexes as antitumor vaccine. J Immunol, 2006. 176(8): p. 4573-80.
35. Wieland, A., et al., Antibody effector functions mediated by Fcgamma-receptors are compromised during persistent viral infection. Immunity, 2015. 42(2): p. 367-378.
36. Tsai, J. F., et al., Circulating immune complexes in chronic hepatitis related to hepatitis C and B viruses infection. Clin Immunol Immunopathol, 1995. 75(1): p. 39-44.
37. Kazmierowski, J. A., D. S. Peizner, and K. D. Wuepper, Herpes simplex antigen in immune complexes of patients with erythema multiforme: presence following recurrent herpes simplex infection. JAMA, 1982. 247(18): p. 2547-50.
38. Chen, X., et al., FcgammaR-Binding Is an Important Functional Attribute for Immune Checkpoint Antibodies in Cancer Immunotherapy. Front Immunol, 2019. 10: p. 292.
39. Chu, T. H., E. F. Patz, Jr., and M. E. Ackerman, Coming together at the hinges: Therapeutic prospects of IgG3. MAbs, 2021. 13(1): p. 1882028.
40. White, A. L., et al., Conformation of the human immunoglobulin G2 hinge imparts superagonistic properties to immunostimulatory anticancer antibodies. Cancer Cell, 2015. 27(1): p. 138-48.
41. Brandsma, A. M., et al., Mechanisms of inside-out signaling of the high-affinity IgG receptor FcgammaRI. Sci Signal, 2018. 11(540).
42. Ganesan, L. P., et al., FcgammaRIIb on liver sinusoidal endothelium clears small immune complexes. J Immunol, 2012. 189(10): p. 4981-8.
43. Turman, J. M., et al., Accelerated Clearance and Degradation of Cell-Free HIV by Neutralizing Antibodies Occurs via FcgammaRIIb on Liver Sinusoidal Endothelial Cells by Endocytosis. J Immunol, 2021. 206(6): p. 1284-1296.
44. Clynes, R., et al., Modulation of immune complex-induced inflammation in vivo by the coordinate expression of activation and inhibitory Fc receptors. J Exp Med, 1999. 189(1): p. 179-85.
45. Wang, X., M. Mathieu, and R. J. Brezski, IgG Fc engineering to modulate antibody effector functions. Protein Cell, 2018. 9(1): p. 63-73.
46. Baker, K., et al., Neonatal Fc receptor for IgG (FcRn) regulates cross-presentation of IgG immune complexes by CD8-CD11b+ dendritic cells. Proc Natl Acad Sci USA, 2011. 108(24): p. 9927-32.
47. <Qiao et al. 2008 FcRn is involved in IC MHC presentation.pdf>.
48. Hubbard, J. J., et al., FcRn is a CD32a coreceptor that determines susceptibility to IgG immune complex-driven autoimmunity. J Exp Med, 2020. 217(10).
49. Vegh, A., et al., FcRn overexpression in transgenic mice results in augmented APC activity and robust immune response with increased diversity of induced antibodies. PLoS One, 2012. 7(4): p. e36286.
50. Rossini, S., et al., V Region of IgG Controls the Molecular Properties of the Binding Site for Neonatal Fc Receptor. J Immunol, 2020. 205(10): p. 2850-2860.
51. Bottermann, M., et al., TRIM21 mediates antibody inhibition of adenovirus-based gene delivery and vaccination. Proc Natl Acad Sci USA, 2018. 115(41): p. 10440-10445.
52. Caddy, S. L., et al., Viral nucleoprotein antibodies activate TRIM21 and induce T cell immunity. EMBO J, 2021. 40(5): p. e106228.
53. Hilchey, S. P., et al., Rituximab immunotherapy results in the induction of a lymphoma idiotype-specific T-cell response in patients with follicular lymphoma: support for a “vaccinal effect” of rituximab. Blood, 2009. 113(16): p. 3809-12.
54. Richards, J. O., et al., Optimization of antibody binding to FcgammaRIIa enhances macrophage phagocytosis of tumor cells. Mol Cancer Ther, 2008. 7(8): p. 2517-27.
55. Alter, G., et al., Immunogenicity of Ad26.COV2.S vaccine against SARS-CoV-2 variants in humans. Nature, 2021. 596(7871): p. 268-272.
56. Corrales-Aguilar, E., et al., A novel assay for detecting virus-specific antibodies triggering activation of Fcgamma receptors. J Immunol Methods, 2013. 387(1-2): p. 21-35.
57. <Soby et al. 2012 HSV-1 induced IP10 expression.pdf>.
58. Thapa, M., et al., CXCL9 and CXCL10 expression are critical for control of genital herpes simplex virus type 2 infection through mobilization of HSV-specific CTL and NK cells to the nervous system. J Immunol, 2008. 180(2): p. 1098-106.
59. Srivastava, R., et al., CXCL10/CXCR3-Dependent Mobilization of Herpes Simplex Virus-Specific CD8(+) TEM and CD8(+) TRM Cells within Infected Tissues Allows Efficient Protection against Recurrent Herpesvirus Infection and Disease. J Virol, 2017. 91(14).
60. Wuest, T. R. and D. J. Carr, Dysregulation of CXCR3 signaling due to CXCL10 deficiency impairs the antiviral response to herpes simplex virus 1 infection. J Immunol, 2008. 181(11): p. 7985-93.
61. Mallery, et al., Antibodies mediate intracellular immunity through tripartite motif-containing 21 (TRIM21). PNAS, 2010. 107(47):19985-19990.
62. Clift et al., A Method for the Acute and Rapid Degradation of Endogenous Proteins. Cell, 2017. 172:1692-1706.
63. Del Gaizo and Payne, A Novel TAT-Mitochondrial Signal Sequence Fusion Protein Is Processed, Stays in Mitochondria, and Crosses the Placenta. Molecular Therapy. 2003. 7(6): 720-730.
64. Argentinian AntiCovid Consortium. Covalent coupling of Spike's receptor binding domain to a multimeric carrier produces a high immune response against SARS-CoV-2. Scientific Reports. 2022. 12:692.
65. Wald A, Timmler B, Magaret A, et al. Effect of Pritelivir Compared With Valacyclovir on Genital HSV-2 Shedding in Patients With Frequent Recurrences: A Randomized Clinical Trial. JAMA 2016; 316:2495-503.
66. Van Wagoner N, Fife K, Leone P A, et al. Effects of Different Doses of GEN-003, a Therapeutic Vaccine for Genital Herpes Simplex Virus-2, on Viral Shedding and Lesions: Results of a Randomized Placebo-Controlled Trial. J Infect Dis 2018; 218:1890-9.
67. Goyal A, Reeves D B, Cardozo-Ojeda E F, Schiffer J T, Mayer B T. Viral load and contact heterogeneity predict SARS-CoV-2 transmission and super-spreading events. Elife 2021; 10:e63537
68. Vezina H E, Balfour H H J, Weller D R, Anderson B J, Brundage R C. Valacyclovir Pharmacokinetics and Exploratory Pharmacodynamics in Young Adults With Epstein-Barr Virus Infectious Mononucleosis. J Clin Pharmacol [Internet] 2010 [cited 2023 Jan. 31]; 50(7):734-42. Available from: http://doi.wiley.com/10.1177/0091270009351884

1. A method for actively immunising a subject against an infectious disease or a malignant disease, wherein said method comprises administering a vaccine comprising an antibody against an antigen correlated with said infectious disease or malignant disease. wherein said antibody comprises at least a portion of an immunoglobulin constant region (Fc) having effector function such that said antibody is capable of eliciting antibody dependent cellular phagocytosis (ADCP) independent from antibody-dependent cellular cytotoxicity (ADCC) and/or complement-dependent cytotoxicity (CDC) when said antibody is bound to said antigen.

2. The method of claim 1, wherein said subject suffers from an acute infectious disease or an acute malignant disease and said subject is having an acute recurrence of said infectious disease or said malignant disease and is suffering from a severe chronic disease with more than 4 recurrences per year, said method comprising administering said vaccine to said subject having said acute recurrence.

3. The method of claim 2, wherein said vaccine is administered by a prime-boost dosage regimen comprising the following administration steps:
(i) administering a first prime administration dosage of the vaccine to the subject during the first week of recurrence, optionally immediately upon start of the first recurrence;
(ii) if said subject has a second recurrence after step (i), administering a boost administration dosage of the vaccine to the subject during the first week of the second recurrence, preferably, optionally immediately upon start of the second recurrence; and
(iii) optionally repeating step (ii) during the first week of every further recurrence to administer further boost administration dosages, optionally immediately upon start of every further recurrence,
wherein one said prime administration and at least one boost administration per year are to be administered.

4-6. (canceled)

7. The method of claim 1, wherein said infectious disease is selected from the group consisting of fungal infections, bacterial infections, protozoan infections and viral infections, optionally wherein said viral infection is a Herpes Simplex Virus (HSV)-associated disease.

8. The method of claim 1, wherein said malignant disease is selected from the group consisting of solid tumors and malignant diseases of the blood/haematooncologic diseases.

9. The method of claim 1, wherein said antigen correlated with said infectious disease is selected from the group consisting of:
Epstein-Barr Virus (EBV) (derived) proteins or domains of proteins, optionally EBNA1, EBNA2, EBNA3A, EBNA3B, EBNA3C, EBNA-LP, LMP1, LMP2A/B, gp350, A73, RPMS1, ZEBRA, Rta, major tegument protein p143, large tegument protein, tegument protein, major capsid protein, minor capsid protein, capsid proteins p18, p23, p40, gN, gp42, gM, gp60, gp78/55, gp150, 53/55kd membrane protein, viral IL-10, gB, gH, gL;
Human Papilloma Virus (HPV) (derived) proteins or domains of proteins, optionally E1, E2, E4, E5, E6, E7, L1, L2;
Human Immunodeficiency Virus 1 or 2 (derived) proteins or domains of proteins, optionally Gag, Pol, Env, gp160, gp120, gp41, CA, MA, p2, p6, NC, IN, RTp66, RTp55, RTp51, PR, Rev, Tat, Nef, Vif, Vpr, Vpu, Vpx;
Herpes Simplex Virus (HSV) 1 or 2 (derived) proteins or domains of proteins, optionally gB, gC, gD, gH, gG, gL, gE, gI, gK, gM, VP1-2, ICP32. ICP0, VP11/12, UL13, vhs, VP16, US3, VP22, ICP34.5, US11, ICP4, DNA polymerase, major capsid protein, helicase, primase, uracil DNA glycosylase, dUTPase, ribonucleotide reductase, large tegument protein;
Human Cytomegalovirus (HCMV) (derived) proteins or domains of proteins encoded by open reading frames (ORFs), optionally UL57, UL55, UL54, UL75, UL86, UL85, UL104, UL97, UL98, UL100, UL105, UL102, UL114, UL115, UL72, UL70, UL69, UL44, UL45, UL48;
Hepatitis A Virus (HAV) (derived) proteins or domains of proteins, optionally VP1, VP2, VP3, VP4, P2-2A, P2-2B, P2-2C, P3-3A, P3-VPg, P3-3Cpro, P3-3Dpol;
Hepatitis B Virus (HBV) (derived) proteins or domains of proteins, optionally M, L, S, polymerase, TP, Spacer, RT, RNaseH, preCore, Core, X, HBeAg;
Hepatitis C Virus (HCV) (derived) proteins or domains of proteins, optionally E1, E2, core, NS1, NS2, NS3, NS4a, NS4b, NS5a, NS5b, precursor polyprotein;
Influenza A Virus (IAV) (derived) proteins or domains of proteins, optionally HA, NA, M2, M1, NP, NS1, NS2/NEP, PA, PB1;
Measles Virus (MV) (derived) proteins or domains of proteins, optionally N, P/C/V, M, F, H, L;
Respiratory Syncytial Virus (RSV) (derived) proteins or domains of proteins, optionally NS1, NS2, N, P, M, SH, G, F, M21, M22, L;
Rotavirus (derived) proteins or domains of proteins, optionally VP1, VP2, VP3, VP4, RdRp, VP5, VP6, VP7, VP8, NSP1, NSP2, NSP3, NSP4, NSP5, NSP6;
Severe acute respiratory syndrome Coronavirus (SARS-Cov) type 1 and 2 (derived) proteins or domains of proteins, optionally ns1, ns2, PLpro, ns4, 3CL, ns6, ns7, ns8, ns9, ns10, RdRp, Hel, ns14, ns15, ns16, S, S-RBD, 3a, E, M, 6, 7a, 7b, 8 N, 9b, 14;
Varizella Zoster Virus (VZV) (derived) proteins or domains of proteins or domains of proteins encoded by open reading frames (ORFs), optionally ORF0 to ORF71 (in total at least 71 proteins);
Human Herpesvirus type 8 (HHV-8) (derived) proteins or domains of proteins, optionally K1, K2, K3, K4, K4.1, K5, K6, K7, K8, K8.1, K9, K10, K10.5 K11, K12, K13, K14, K15, gB, gL, gH, and proteins encoded in ORFs including but not limited to ORF2, ORF9, ORF10, ORF16, ORF18, ORF24, ORF30, ORF31, ORF34, ORF66, ORF21, ORF23, ORF25, ORF26, ORF65, ORF33, ORF34, ORF35, ORF36, ORF37, ORF38, ORF39, ORF40, ORF41, ORF42, ORF45, ORF49, ORF50, ORF52, ORF53, ORF55, ORF57, ORF59, ORF67, ORF69, ORF70, ORF72, ORF73, ORF74, ORF75;
Human Herpesvirus type 6 (HHV-6) (derived) proteins or domains of proteins encoded by ORFs, optionally DR1, DR6, DR7/U1, U2, U3, U4, U7, U10, U11, U12, U13, U14, U15, U17, U18, U19, U20, U21, U22, U23, U24, U25, U26, U27, U28, U29, U30, U31, U32, U33, U34, U35, U36, U37, U38, U39, U40, U41, U42, U43, U44, U45, U46, U47, U48, U49, U50, U51, U52, U53, U54, U55, U56, U57, U58, U59, U61, U62, U63, U64, U65, U66, U69, U70, U71, U72, U73, U74, U75, U76, U77, U79, U81, U82, U83, U85, U86, U88, U90, U91, U94, U95, U100;
Rabies Virus (derived) proteins or domains of proteins, optionally N, P, M, G, L;
Mumps Virus (derived) proteins or domains of proteins, optionally N, V/P, M, F, HN, L; and
Rhinovirus (derived) proteins or domains of proteins, optionally VP1, VP2, VP3, VP4, P2-2A, P2-2B, P2-2C, P3-3A, P3-VPg, P3-3C, P3-3D; or
said antigen correlated with said malignant disease is selected from the group consisting of:
tumor-associated antigens, preferably carbonic anhydrase IX, CCCL19, CCCL21, CSAp, CD1, CD1a, CD2, CD3, CD4, CD5, CD8, CD11A, CD14, CD15, CD16, CD18, CD19, IGF-1R, CD20, CD21, CD22, CD23, CD25, CD29, CD30, CD32b, CD33, CD37, CD38, CD40, CD40L, CD45, CD46, CD52, CD54, CD55, CD59, CD64, CD66a-e, CD67, CD70, CD74, CD79a, CD80, CD83, CD95, CD126, CD133, CD138, CD147, CD154, AFP, PSMA, CEACAM5, CEACAM6, B7, ED-B of fibronectin, Factor H, FHL-1, Flt-3, folate receptor, GROB, HER2, HMGB-1, hypoxia inducible factor (HIF), HM1.24, insulin-like growth factor-1 (ILGF-1), IFN-γ, IFN-α, IFN-β, IL-2, IL-4R, IL-6R, IL-13R, IL-15R, IL-17R, IL-18R, IL-6, IL-8, IL-12, IL-15, IL-17, IL-18, IL-25, IP-10, mCRP, MCP-1, MIP-1A, MIP-1B, MIF, MUC1, MUC2, MUC3, MUC4, MUC5ac, PAM4 antigen, NCA-95, NCA-90, NY-ESO-1, Ia, HM1.24, EGP-1, EGP-2, HLA-DR, tenascin, Le(y), RANTES, T101, TAC, Tn antigen, Thomson-Friedenreich antigens, tumor necrosis antigens, TNF-α, TRAIL receptor (R1 and R2), VEGFR, EGFR, PlGF, complement factors C3, C3a, C3b, C5a, C5, cancer testis (CT) antigens SPAG9, CT9, CT10, LAGE, MAGE-B5, MAGE-B6, MAGE-C2, MAGE-C3, MAGE-D, HAGE, SAGE and an oncogene product.

10. The method of claim 1, wherein said antibody is a monoclonal antibody, optionally wherein said monoclonal antibody is a humanized or fully human antibody.

11. (canceled)

12. The method of claim 2 wherein said active immunization elicits a persistent or long-term immunity against said infectious disease or said malignant disease in said subject suffering from said acute infectious disease or said acute malignant disease.

13. The method of claim 1, wherein said infectious disease is Herpes Simplex Virus (HSV)-associated disease, and wherein said antibody is an anti-HSV antibody, and said subject suffers from an acute HSV-associated disease, optionally an acute HSV infection.

14. The method of claim 13, comprising administering said vaccine wherein said subject has at least one acute recurrence of said disease and is suffering from a severe chronic HSV-associated disease, optionally a severe chronic HSV infection, with more than 4 recurrences per year.

15-20. (canceled)

21. The method of claim 13, wherein said anti-HSV antibody recognizes the glycoprotein B (gB) of HSV-1 and/or HSV-2.

22. The method of claim 13, wherein said antibody is an antibody comprising the complementarity determining regions V_{H}CDR1 having the amino acid sequence of SEQ ID NO: 1, V_{H}CDR2 having the amino acid sequence of SEQ ID NO: 2, V_{H}CDR3 having the amino acid sequence of SEQ ID NO: 3, V_{L}CDR1 having the amino acid sequence of SEQ ID NO: 4, V_{L}CDR2 having the amino acid sequence of SEQ ID NO: 5, and V_{L}CDR3 having the amino acid sequence of SEQ ID NO:6, or an antibody comprising a V_{H} region having the amino acid sequence of SEQ ID NO:9 and a V_{L} having the amino acid sequence of SEQ ID NO:10.

23. The method of claim 22, wherein said antibody comprises a framework sequence having an amino acid sequence with at least 70% sequence identity to the amino acid residues shown in positions 1 to 30, 38 to 51, 68 to 99, and 112 to 122 of SEQ ID NO: 7 and in positions 1 to 23, 41 to 55, 63 to 94, and 104 to 114 of SEQ ID NO: 8.

24-25. (canceled)

26. The method of claim 13, wherein said antibody is to be administered intravenously, topically, intradermally, subcutaneously, intra-cutanously, intramuscular an/or intrathecal.

27. The method of claim 13, wherein said disease is selected from the group consisting of Herpes simplex labialis, Herpes simplex genitalis, chronic or disseminated cutaneous herpes simplex infection, Herpes gladiatorum, Eczema herpeticum, Herpes keratoconjunctivitis, Herpes neonatorum, Alzheimer disease (AD), HSV pneumonia, Bell's palsy, Herpes esophagitis, Herpesviral encephalitis and Herpesviral meningitis, Herpetic sycosis, Herpes withlow, Herpes gingivostomatitis, presence of an oral recidive, presence of a genital recidive, eczema herpeticatum, herpes neonatorum, immune deficiency, immunocompromised patients, resistance against a virusstatic agent, encephalitis, meningitis, meningoencephalitis, eye infections, and/er generalized HSV infections.

28. The method of claim 13, wherein the anti-HSV antibody is a full-length antibody/complete antibody.

29. The method of claim 13, wherein said anti-HSV antibody comprises an Fc region having the amino acid sequence of SEQ ID NO: 53.

30. The method of claim 13, wherein said anti-HSV antibody comprises a V_{H} region having the amino acid sequence of SEQ ID NO:9, a V_{L} region having the amino acid sequence of SEQ ID NO:10, and an Fc region having the amino acid sequence of SEQ ID NO: 53.
